(19) United States
(12) Patent Application Publication
(10) Pub. No.: US 2013/0081158 A1
(43) Pub. Date: Mar. 28, 2013

(54) Sorghum Grain Shattering Gene and Uses Thereof in Altering Seed Dispersal

(71) Applicant: University of Georgia Research Foundation, Inc., Athens, GA (US)

(72) Inventors: Andrew Paterson, Arnoldsville, GA (US); Haibao Tang, Rockville, MD (US)

(73) Assignee: UNIVERSITY OF GEORGIA RESEARCH FOUNDATION, INC., Athens, GA (US)

(21) Appl. No.: 13/664,063

(22) Filed: Oct. 30, 2012

Related U.S. Application Data
(63) Continuation of application No. PCT/US2012/045973, filed on Jul. 9, 2012.
(60) Provisional application No. 61/505,344, filed on Jul. 7, 2011.

(51) Int. Cl. C12N 15/82; C07K 14/415

(52) U.S. Cl. CPC C12N 15/8261; C07K 14/415
USPC 800/320; 536/23.6; 530/372; 530/324; 435/320.1; 435/419

(57) ABSTRACT

Compositions and methods relating to identification of the sorghum grain shattering gene (Sh1) for use in modulating fruit dehiscence in a plant are provided. For example, methods are provided for developing genetically modified plant varieties in which the natural seed dispersal process is delayed. Likewise, methods are provided for treating a plant in order to delay fruit dehiscence in the plant. Screening methods are also provided for identifying chemical agents that can modify natural seed dispersal.

### CROSS-REFERENCE TO RELATED APPLICATIONS

This application is a bypass continuation of PCT/US2012/045973 filed under the Patent Cooperation Treaty on Jul. 9, 2012, which claims the benefit of and priority to U.S. Provisional Application No. 61/505,344, entitled “Sorghum Grain Shattering Gene And Uses Thereof In Delaying Seed Dispersal” filed Jul. 7, 2011, and where permissible is incorporated by reference in its entirety.

### STATEMENT REGARDING FEDERALLY SPONSORED RESEARCH OR DEVELOPMENT

This invention was made with Government Support under Agreements 96-35300-3924 and 01-35301-10595 awarded by the United States Department of Agriculture. The Government has certain rights in the invention.

### REFERENCE TO SEQUENCE LISTING

The Sequence Listing submitted Oct. 30, 2012 as a text file named “UGA_{—}1538_CON_Sequence_Listing.txt,” created on Oct. 30, 2012, and having a size of 73,728 bytes is hereby incorporated by reference pursuant to 37 C.F.R. §1.52(e)(5).

### FIELD OF THE INVENTION

The invention is generally related to plant genetic engineering. In particular, the invention relates to methods and compositions that modulate fruit or seed dehiscence in plants.

### BACKGROUND OF THE INVENTION

Cultivated sorghum (Sorghum bicolor) is a leading cereal in agriculture, ranking fifth in importance among the worlds' grain crops. Sorghum is used for food, feed, fodder, and the production of ethanol. Sorghum plants are more tolerant to drought and heat than most other grasses, making it an ideal staple food in arid African countries. Among the more than 20 species within the Sorghum genus, S. halepense, S. almum and hybrids of these to the cultivated S. bicolor, collectively known as “Johnson grass”, are notorious weeds affecting crop yields (Draye, et al., Plant Physiol, 125:1325-41 (2001)).

The domestication of sorghum started in Africa and then was carried to Europe and Asia before North America. Wild species of sorghum are found as early as 8000 years ago in the Nilotic regions of southern Egypt and Sudan, but the location of its true domestication within East Africa is still speculative (Dahlberg, African Crop Science Journal, 3:143-51 (1995)). Members of the Sorghum genus (Sorghums) disperse by two major ways: vegetative reproduction through subterranean rhizomes and seed dispersal by shattering. Although disadvantageous in the wild habitat, non-shattering sorghums are thought to have been selected during domestication because humans could more efficiently harvest grains that remained attached to the plant. During plant development, the shattering of seeds involves the formation of an abscission layer and is considered a process of programmed senescence.

The pathway involving the formation of the abscission layer is well characterized in some eudicot species. SHATTERPROOF genes SHP1 and SHP2 have been shown to specify valve margin cell identities in Arabidopsis (Liljegren, et al., Nature, 404:766-70 (2000)). The expression of the SHP genes are reinforced through negative regulation from FRUITFUL (FUL) in valve development (Ferrandiz, et al., Science, 289:436-438 (2000)) and REPLUMLESS (RPL) in the replum (Roeder, et al., Curr Biol, 13:1630-35 (2003)). However, the botanical origin of the abscission layer in Arabidopsis is clearly different from that of rice or other cereals. The layer contributing to seed shattering studied in Arabidopsis is located at the valve-replum boundary and does not correspond to that of cereals which is at the base of the pedicel. Therefore, it remains doubtful whether orthologous genes are implicated in the seed dispersal mechanisms of dicots and cereals, respectively.

Two major genes that contribute to the shattering trait in rice (Oryza sativa ssp.) were identified—qSH1 and sh4, controlling 68% and 69% of the phenotypic variance in the studied crosses, respectively (Konishi, et al., Science, 312:1392-96 (2006); Li, et al., Science, 311:1936-1939 (2006)). In both cases, the non-shattering phenotype is caused by the absence of the abscission layer (or dehiscence zone), though sh4 shows a change of protein function while qSH1 shows a change in expression pattern as a result of domestication (Konishi, et al., Science 312:1392-96 (2006); Li, et al., Science, 311:1936-1939 (2006)). The fixation of sh4 occurred very early in rice domestication with the domesticated allele occurring in both indica and japonica, while qSH1 is much more recent and is present only within temperate japonica individuals (Konishi, et al., Plant Cell Physiol, 49:1283-93 (2008); Zhang, et al., New Phytol., 184(3):708-20 (2009)). In wheat, QTLs that are responsible for nonbrittle rachis are located in the homeologous regions of chromosome 3A (Br2), 3B (Br3) and 3D (Br1) (Nalam, et al., Theor Appl Genet, 116:135-45 (2007); Nalam, et al., Theor Appl Genet, 112:373-81 (2006)). Comparative mapping hinted that this part of the chromosomal regions might correspond to the orthologous region in barley, controlled by two tightly linked loci, Btr1 and Btr2, but do not appear to correspond to the region in other major cereals (Nalam, et al., Theor Appl Genet, 116:135-45 (2007); Nalam, et al., Theor Appl Genet, 112:373-81 (2006)). Indeed, many of these genes in different cereal crops do not appear to be in corresponding (orthologous) chromosomal locations, therefore there may be multiple pathways responsible for seed dispersal in the grasses (Li, et al., Funct Integr Genomics, 6:300-09 (2006)). Steady progress in rice notwithstanding, many more rice genes that control shattering exist (Paterson, et al., Science 269:1714-18 (1995)) but have not yet been identified, therefore the above hypothesis remains to be tested. Additionally, since sorghum and maize are closer to one another than to rice, the shattering loci between the two panicoid species may still partially correspond (Paterson, et al., Science 269:1714-18 (1995)).

Seed/grain losses due to shattering remain a significant economic problem in common cereal crops such as wheat, oat, barley, and rice; forages such as bahiagrass, dallisgrass, kleingrass, guineagrass, reed canarygrass, orchardgrass, ricegrass, foxtail, and vetch; legumes such as soybean, lentil, and chickpea; oilseeds such as canola; vegetables such as onion and carrot; and specialty crops such as caraway, hemp, and sesame. Moreover, economical large-scale cultivation of many prospective new crops would be greatly facilitated by suppression of shattering—some examples include wild rice, birdsfoot trefoil, castor, oilseed spurge, Veronica and others.

Moreover, shattering contributes to the dissemination of agricultural weeds such as Johnson grass, wild oat, proso millet, and red rice. If growth regulators could be identified that induced premature shattering, it could cause dispersal before seeds are viable, reducing the weed “seed reservoir” in the soil.

It is an object of the invention to identify genes that regulate the shattering process in Sorghum grains.

It is a further object of the invention to provide genetically modified plants with modified seed shattering.

It is still a further object of the invention to provide a means for identifying chemical treatments that can modify natural seed dispersal.

It is yet a further object of the invention to provide a means for identifying genes that regulate the seed shattering process in other plants.

### SUMMARY OF THE INVENTION

Compositions and methods relating to the sorghum grain shattering gene (Sh1) are provided. One embodiment provides an isolated nucleic acid having a nucleic acid sequence at least 90% identical to SEQ ID NO:1, 2, 3, 4, 5, 6, 7, 8, 9, 10, or 11 or a nucleic acid sequence encoding SEQ ID NO: 12, 13, 14, 15, 16, or 17, or a complement thereof. Also disclosed is an isolated nucleic acid having a nucleic acid sequence that hybridizes under stringent conditions to a polynucleotide consisting of the nucleic acid sequence SEQ ID NO: 1, 2, 3, 4, 5, 6, 7, 8, 9, 10, or 11 or a nucleic acid sequence encoding SEQ ID NO: 5, 6, 7, 8, 9, or 10, or a complement thereof.

Another embodiment provides a transgenic plant or transgenic plant cell including an expression control sequence operably linked to a nucleic acid sequence that silences expression of a polynucleotide having a nucleic acid sequence SEQ ID NO: 1, 2, 3, 4, 5, 6, 7, 8, 9, 10, or 11, or a nucleic acid sequence encoding SEQ ID NO: 12, 13, 14, 15, 16, or 17, or a complement thereof. For example, in some embodiments, transcription of the nucleic acid in the plant or plant cell results in a double-stranded RNA molecule capable of reducing the expression of a gene endogenous to the plant, wherein the gene is involved in plant dehiscence. The double-stranded RNA can include a nucleic acid sequence at least 90% identical to SEQ ID NO: 1, 2, 3, 4, 5, 6, 7, 8, 9, 10, or 11 or a nucleic acid sequence encoding SEQ ID NO: 12, 13, 14, 15, 16, or 17 or a complement thereof. In preferred embodiments, the disclosed transgenic plant has reduced seed shattering compared to a non-transgenic plant of the same species while maintaining an agronomically relevant threshability. Representative transgenic plants include transgenic sugarcane, maize, Sorghum, finger millet, switchgrass, Miscanthus, and amaranth.

Also disclosed is an agricultural method, involving planting a disclosed transgenic plant or sowing seeds from a disclosed transgenic plant; growing the plants until the seeds are mature; and harvesting seeds by threshing with a combine harvester.

Also disclosed are methods of reducing or delaying fruit dehiscence in a plant, involving introducing to the plant a nucleic acid sequence that silences expression of a polynucleotide having a nucleic acid sequence SEQ ID NO:1, 2, 3, 4, 5, or 6, or a nucleic acid sequence encoding SEQ ID NO:12, 13, 14, or 15; or that increases expression of a nucleic acid sequence SEQ ID NO:7, 8, 9, 10, or 11, or a nucleic acid sequence encoding SEQ ID NO:16 or 17; or combinations thereof. As a result of this method, the transgenic plant preferably has reduced or delayed seed shattering compared to non-transgenic (e.g., wild-type) plant of the same species. Preferably, the transgenic plant retains agronomically relevant threshability.

Also disclosed are methods of increasing or accelerating fruit dehiscence in a plant, involving introducing to the plant a nucleic acid sequence that silences expression of a polynucleotide having a nucleic acid sequence SEQ ID NO: 7, 8, 9, 10, or 11, or a nucleic acid sequence encoding SEQ ID NO: 16 or 17; or that increases expression of a nucleic acid sequence SEQ ID NO: 1, 2, 3, 4, 5, or 6, or a nucleic acid sequence encoding SEQ ID NO: 12, 13, 14, or 15; or combinations thereof. As a result of this method, the transgenic plant preferably has increased or accelerated seed shattering compared to non-transgenic (e.g., wild-type) plant of the same species.

### BRIEF DESCRIPTION OF THE DRAWINGS

FIG. 1 is a graph showing synonymous (x-axis, Ks) and non-synonymous (y-axis, Ka) substitutions between orthologous pairs of genes from S. bicolor (non-shattering) and S. propinquum (shattering), in the region containing the shattering gene.

FIG. 2 is a diagram illustrating the distributions of repeats and genes in the region containing the shattering gene of S. bicolor.

FIG. 3 is a diagram showing aligned positions for Sorghum propinquum BACs. The line segments represent aligned contigs within each BAC, with lines showing alignments with the same orientations and alignments with the opposite orientations. The dotted lines represent the genetic markers flanking (SOG0251, SOG1273) or co-segregating (SOG0128) with Sh1.

FIG. 4 is a graph showing breaking force (g) as a function of time after flowering (days) for two “non-shattering” varieties of sorghum grain: (AN04 (#14), solid line) and (AP03 (#16), dotted line).

FIG. 5 is a graph showing progression of required breaking force (g) as a function of time after flowing (days) for two “shattering” varieties of sorghum grain: (BP10 (#6), solid line) and (BP11 (#22), dotted line).

FIG. 6 is a graph showing strength of linkage disequilibrium (r²) as a function of the distance between sites (bp). The curve is the logarithmic fit of the data, and the distances at 511 bp and 14406 bp is shown as the distance where r² drops to 50% and 20%, respectively.

FIG. 7 is a pairwise LD matrix of the SNPs genotyped in this study, as generated by TASSEL (Bradbury et al. 2007 Bioinformatics 23: 2633-35). The markers are ordered according to their physical positions in the shattering region. The upper right matrix plots the pairwise r² score (ranging from 0 to 1, 1 means perfect LD). The lower left portion of the matrix plots the P-value from the Fisher's exact test (two-alleles) or test of independence (multiple alleles).

FIG. 8 is a graph showing the strength of associations (−log₁₀P) as a function of position in Sorghum chromosome 1 (Mb).

FIG. 9 is a diagram illustrating phylogenetic relationship among haplotypes of the individuals in the study. Boxed labels are the accessions that shatter; Circled labels are the accessions that don't shatter. #0 is S. bicolor line BTX623, #20 is S. propinquum, the two parents used in the linkage mapping.

FIG. 10A is a series of panels illustrating the fine mapping procedure used to narrow down the range of the candidate Sh1 gene in sorghum. Panels from top to bottom represent: the RFLP markers used in the study, which are shown are either flanking (SOG1273, SOG0251) or co-segregating (SOG0128) with the shattering trait (top panel); the delineated region (chr1: 11.5 Mb-12.2 Mb) which was subject to fine mapping with amplicon-based SNP markers, along with the strength of associations at the tested SNP sites in the shattering region (second panel from the top); four SNPs (P7E9, P3H11, P8F9, P4C3) were tested to be significantly associated with the seed shattering trait at P<0.001 (third panel from the top); two genes (Sb01g012870 and Sb01g012880) fall inside the vicinity of the SNP sites that showed highest association (bottom panel).

FIG. 10B is an alignment of O. sativa ortholog (Os03g0657400) (SEQ ID NO:18), S. propinquum allele (Sh1.fgenesh) (SEQ ID NO:12) and S. bicolor allele (Sb01g012870) (SEQ ID NO:16). The WRKY domain is between position 51 and 104. Note that the S. propinquum and S. bicolor alleles differ at the position of the start codon, resulting in a shorter S. bicolor protein.

FIG. 11A is a multiple gene alignment diagram showing the orthologs of Sh1 from five grasses: S. bicolor (Sb01g012870) (SEQ ID NO:16); S. propinquum (Sh1.fgenesh) (SEQ ID NO:12); Zea mays (GRMZM2G149219) (SEQ ID NO:19); Zea mays (GRMZM2G161411) (SEQ ID NO:20); Setaria italica (Si038001m) (SEQ ID NO:21); Setaria italica (Si038955m) (SEQ ID NO:22); Brachypodium dist (Bradi1g113210) (SEQ ID NO:23); and O. sativa (Os03g0657400) (SEQ ID NO:18). The WRKY domain is located between columns 62 and 115 (as shown) and is perfectly matching between S. propinquum and S. bicolor. Consistent with the alignment in FIG. 10B, the S. propinquum and S. bicolor alleles differ at the position of start codon, resulting in a shorter S. bicolor protein. There is only one copy each in sorghum, rice, Brachypodium, but two copies in maize and Setaria. The column highlighted in the solid box marks the aligned position for start codons of the “short” proteins.

FIG. 11B is a neighbor-joining tree among the selected Sh1 homologs. The number next to the branch nodes are bootstrap values (with 500 bootstrap samples). Exon structure for individual gene homologs is shown next to the label (with coding exons in blocks) as well as the size of the protein. The grass proteins selected are direct orthologs to Sh1.

FIG. 12A is a line graph showing Measurement of Breaking Tensile Strength (BTS) (Force (grams)) of inflorescence from shattering type sorghum at different developmental stages. For each stage ten individual florets were tested from two different panicles. Bars represent ±1 SE (n=2).

FIG. 12B is a line graph showing Measurement of Breaking Tensile Strength (BTS) (Force (grams)) of inflorescence from non-shattering type sorghum at different developmental stages. For each stage ten individual florets were tested from two different panicles. Bars represent ±1 SE (n=2).

FIG. 13 is a pictograph of the results of gel electrophoresis following semi-quantitative RT-PCR expression profiling of Sh1 gene (SbWRKY) in shattering and non-shattering sorghum along with another candidate gene (SbTATA). SbActin was used as a loading control. S=shattering, N=non-shattering; Inf. Not Em.=inflorescence still in flag leaf, Inf. Just em.=inflorescence just emerging from flag leaf, Inf. With anth.=after anther dehiscence.

### DETAILED DESCRIPTION OF THE INVENTION

### I. Definitions

Before describing the various embodiments, it is to be understood that the invention is not limited in its application to the details of construction and the arrangement of the components set forth in the following description. Other embodiments can be practiced or carried out in various ways. Also, it is to be understood that the phraseology and terminology employed herein is for the purpose of description and should not be regarded as limiting.

Unless otherwise indicated, the disclosure encompasses conventional techniques of plant breeding, microbiology, cell biology and recombinant DNA, which are within the skill of the art. See, e.g., Sambrook and Russell, Molecular Cloning: A Laboratory Manual, 3rd edition (2001); Current Protocols In Molecular Biology [(F. M. Ausubel, et al. eds., (1987)]; Plant Breeding: Principles and Prospects (Plant Breeding, Vol 1) M. D. Hayward, N. O. Bosemark, I. Romagosa; Chapman & Hall, (1993); Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)].

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience., 1999; and Robert A. Meyers (ed.), Molecular Biology and Biotechnology, a Comprehensive Desk Reference, published by VCH Publishers, Inc., 1995; Sambrook and Russell. (2001) Molecular Cloning: A Laboratory Manual 3rd. edition, Cold Spring Harbor Laboratory Press.

To facilitate understanding of the disclosure, the following definitions are provided:

The term “plant” is used in it broadest sense. It includes, but is not limited to, any species of woody, ornamental or decorative crop or cereal, and fruit or vegetable plant. It also refers to a plurality of plant cells that are largely differentiated into a structure that is present at any stage of a plant's development. Such structures include, but are not limited to, a fruit, shoot, stem, leaf, flower petal, etc.

The term “fruit” refers to a structure of a plant that contains its seeds as well as the grain of a crop, such as a cereal, known as a caryopsis fruit.

The terms “seed shattering,” “pod shattering,” and fruit “dehiscence” refer to the process by which a fruit opens to release its seeds. The fruit contains two carpels joined margin to margin. The suture between the margins forms a thick rib called the replum. As seed maturity approaches, the two valves separate progressively from the replum, along designated lines of weakness in the fruit, eventually resulting in the shattering of the seeds that were attached to the replum. The dehiscence zone defines the exact location of the valve dissociation.

The term “delayed” dehiscence is used broadly to encompass both seed dispersal that is significantly postponed as compared to the seed dispersal in a corresponding control plant, and to seed dispersal that is completely precluded, such that fruits never release their seeds unless there is human or other intervention. It is recognized that there can be natural variation of the time of seed dispersal within a plant species or variety. However, a “delay” in the time of seed dispersal can be identified by sampling a population of plants and determining that the normal distribution of seed dispersal times is significantly later, on average, than the normal distribution of seed dispersal times. Thus, production of the disclosed plants provides a means to skew the normal distribution of the time of seed dispersal from pollination, such that seeds are dispersed, on average, at least about 1%, 2%, 5%, 10%, 30%, 50%, 100%, 200% or 500% later than in the corresponding control plant species.

The term “indehiscent” refers to plants where seed dispersal is completely precluded, such that the plants never release their seeds unless there is human or other intervention.

The term “threshing” refers to the use of physical force to release seeds from a fruit. The term “threshability” refers to the resistance of a fruit to opening along the dehiscence zone and releasing its seeds upon application of physical forces. The term “an agronomically relevant” threshability refers to the ability to use threshing to achieve complete release of the seeds without damage to the seeds. For example, threshability can be determined using a random impact tests (RITs).

The term “non-naturally occurring plant” refers to a plant that does not occur in nature without human intervention. Non-naturally occurring plants include transgenic plants and plants produced by non-transgenic means such as plant breeding.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

The term “plant material” refers to leaves, stems, roots, flowers or flower parts, fruits, pollen, egg cells, zygotes, seeds, cuttings, cell or tissue cultures, or any other part or product of a plant.

The term “plant organ” refers to a distinct and visibly structured and differentiated part of a plant such as a root, stem, leaf, flower bud, or embryo.

The term “plant cell” refers to a structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

The term “plant cell culture” refers to cultures of plant units such as, for example, protoplasts, cell culture cells, cells in plant tissues, pollen, pollen tubes, ovules, embryo sacs, zygotes and embryos at various stages of development.

The term “transgenic plant” refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

The term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “orthologous genes” or “orthologs” refer to genes that have a similar nucleic acid sequence because they were separated by a speciation event.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components.

An “isolated” nucleic acid molecule or polynucleotide is a nucleic acid molecule that is identified and separated from at least one contaminant nucleic acid molecule with which it is ordinarily associated in the natural source. The isolated nucleic can be, for example, free of association with all components with which it is naturally associated. An isolated nucleic acid molecule is other than in the form or setting in which it is found in nature.

As used herein, the term “linkage disequilibrium” or “LD” refers to the situation in which the alleles for two or more loci do not occur together in individuals sampled from a population at frequencies predicted by the product of their individual allele frequencies. Markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait.

As used herein, the term “locus” refers to a specific position along a chromosome or DNA sequence. Depending upon context, a locus could be a gene, a marker, a chromosomal band or a specific sequence of one or more nucleotides.

The term “vector” refers to a replicon, such as a plasmid, phage, or cosmid, into which another DNA segment may be inserted so as to bring about the replication of the inserted segment. The vectors can be expression vectors.

The term “expression vector” refers to a vector that includes one or more expression control sequences

The term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “promoter” refers to a regulatory nucleic acid sequence, typically located upstream (5′) of a gene or protein coding sequence that, in conjunction with various elements, is responsible for regulating the expression of the gene or protein coding sequence. The promoters suitable for use in the constructs of this disclosure are functional in plants and in host organisms used for expressing the disclosed polynucleotides. Many plant promoters are publicly known. These include constitutive promoters, inducible promoters, tissue- and cell-specific promoters and developmentally-regulated promoters. Exemplary promoters and fusion promoters are described, e.g., in U.S. Pat. No. 6,717,034, which is herein incorporated by reference in its entirety.

A nucleic acid sequence or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking can be accomplished by ligation at convenient restriction sites. If such sites do not exist, synthetic oligonucleotide adaptors or linkers are used in accordance with conventional practice.

“Transformed,” “transgenic,” “transfected” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

The term “endogenous” with regard to a nucleic acid refers to nucleic acids normally present in the host.

The term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

The term “percent (%)sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotide or amino acid sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “suppressed,” “silenced,” or “decreased” Sh1 gene expression encompasses the absence of Sh1 gene expression or encoded protein levels in a plant, as well as gene expression that is present but reduced as compared to the level of Sh1 gene expression in a wild type plant. The term “suppressed” also encompasses an amount of Sh1 protein that is equivalent to wild type Sh1 expression, but where the Sh1 protein has a reduced level of activity.

Small RNA molecules are single stranded or double stranded RNA molecules generally less than 200 nucleotides in length. Such molecules are generally less than 100 nucleotides and usually vary from 10 to 100 nucleotides in length. In a preferred format, small RNA molecules have 11, 12, 13, 14, 15, 16, 17, 18, 19, 20, 21, 22, 23, 24, 25, 26, 27, 28, 29 or 30 nucleotides. Small RNAs include microRNAs (miRNA) and small interfering RNAs (siRNAs). mRNAs are produced by the cleavage of short stem-loop precursors by Dicer-like enzymes; whereas, siRNAs are produced by the cleavage of long double-stranded RNA molecules. MiRNAs are single-stranded, whereas siRNAs are double-stranded.

The term “siRNA” means a small interfering RNA that is a short-length double-stranded RNA that is not toxic. Generally, there is no particular limitation in the length of siRNA as long as it does not show toxicity. “siRNAs” can be, for example, 15 to 49 bp, preferably 15 to 35 bp, and more preferably 21 to 30 bp long. Alternatively, the double-stranded RNA portion of a final transcription product of siRNA to be expressed can be, for example, 15 to 49 bp, preferably 15 to 35 bp, and more preferably 21 to 30 bp long. The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. Nonpairing portions can be contained to the extent that they do not interfere with siRNA formation. The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number.

The terminal structure of siRNA may be either blunt or cohesive (overhanging) as long as siRNA can silence, reduce, or inhibit the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 bp double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the “siRNA” is not necessarily the cut off structure at both ends as described above, and may have a stem-loop structure in which ends of one side of double-stranded RNA are connected by a linker RNA. The length of the double-stranded RNA region (stem-loop portion) can be, for example, 15 to 49 bp, preferably 15 to 35 bp, and more preferably 21 to 30 bp long. Alternatively, the length of the double-stranded RNA region that is a final transcription product of siRNAs to be expressed is, for example, 15 to 49 bp, preferably 15 to 35 bp, and more preferably 21 to 30 bp long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA or viral RNA, or an artificial RNA molecule.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Third Edition, Cold Spring Harbor, N.Y. (2000).

### II. Compositions

Compositions and methods for controlling seed dispersal in the plant by modulating fruit dehiscence are provided. The methods can involve modulating the activity of the endogenous gene responsible for seed shattering activity in the plant.

For example, the methods can involve suppressing the expression of an endogenous gene orthologous to sorghum grain shattering gene (Sh1). Thus, the methods can involve introducing to the plant a composition that inhibits shattering gene (Sh1) activity in a Sorghum propinquum plant.

Alternatively, the methods can involve promoting the expression of an endogenous gene orthologous to sorghum grain shattering gene (Sh1). Thus, the methods can involve introducing to the plant a composition that promotes shattering gene (Sh1) activity in a Sorghum propinquum plant.

The term “Sh1” refers to the gene product disclosed herein that is responsible for seed shattering (dehiscence) in wild-type sorghum plants. Nucleic acid sequences for Sh1 genes in Sorghum bicolor and Sorghum propinquum are provided.

It is understood that the skilled artisan can identify orthologous sequences in other Sorghum species for use in the present compositions and methods. For example, Sh1 genes from Sorghum almum, Sorghum amplum, Sorghum angustum, Sorghum arundinaceum, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum purpureosericeum, Sorghum stipoideum, Sorghum timorense, Sorghum trichocladum, Sorghum versicolor, Sorghum virgatum, and Sorghum vulgare can be identified and used in the disclosed methods.

Some Sorghum bicolor genotypes are non-shattering members of the Sorghum genus. Thus, it is understood that the skilled artisan can avoid Sh1 orthologous genes that are non-shattering. Likewise, the skilled artisan can use the guidance provided by the sequence comparisons to identify variants of the Sh1 genes that can generate the shattering phenotype.

Also disclosed is a transgenic plant having a nucleic acid molecule, or antisense constructs thereof, encoding an Sh1 gene product operatively linked to an expression control sequence. In some embodiments, the expression control sequence is a heterologous expression control sequence. For example, disclosed is a transgenic plant characterized by delayed seed dispersal, wherein the cells of the plant express a nucleic acid molecule encoding an Sh1 gene product, or antisense construct thereof, that is operatively linked to an expression control sequence, such as a heterologous expression control sequence.

A. Nucleic Acids

1. Shattering Sh1 Gene

Disclosed are polynucleotides having a shattering Sh1 gene from a sorghum plant. The Sorghum plant can be S. propinquum. Sequences for the Sh1 gene in S. propinquum are provided.

It is understood that where coding sequences for an Sh1 gene is provided, also provided are the non-coding sequences that are known or can be identified to correspond to the coding sequence that is provided. For example, where an Sh1 gene is provided, also provided for use in the disclosed compositions and methods is the 5′ untranslated region (UTR), which contains the endogenous promoter for the Sh1 gene. Although not expressly recited, it is understood that the skilled artisan can identify these sequences with routine skill and experimentation based on the sequences that are provided.

The coding sequence, without introns, of the shattering Sh1 gene as it is found in S. propinquum can include the nucleic acid sequence:

```
    1 ATGGATTCAA GCTCACAGCC CGGCGCAATT GATACATGCA GAGGGAGCGG AGGAGGAGGA
  61 GATAGAAACC AAAGGGAGGA GGACGCGGCG GCGGCGGCGG CGGCAGAGGC CGGCTACGGC
121 AGGCAGCTGG TGATTCCCGA GGACGGGTAC GAGTGGAAGA AGTACGGCCA GAAGTTCATC
181 AAGAACATCC AGAAAATCAG GAGCTACTTC CGGCTACGGC ACAAGCTGTG CGGCGCCAAG
241 AAGAAGGTGG AGTGGCACCC GCGGGACCCC AGCGGCGACC TCCGCATCGT CTACGAGGGC
301 GCGCACCAGC ACGGCGCCCC GGCGGCGGCG GCTCCTCCCG GTCCCGGCGG CCAGCATCAG
361 GGCGGCGGCG CCTCCGACTT CAACAGATAC GAGCTGGGCG CGCAGTACTT CGGCGGGGCC
421 GGCCGGTCGC ATTGA
```

(SEQ ID NO:1, Sp01g012870, S. propinquum), or a variant thereof having at least 90%, 95%, or more sequence identity to SEQ ID NO:1.

In some embodiments, the coding sequence, including introns, of the shattering Sh1 gene in S. propinquum can include the nucleic acid sequence:

```
      1 ATGGATTCAA GCTCACAGCC CGGCGCAATG TATGCATCTC TCTCTCTCTC TCTCTCTCTC
    61 TCTCTCTCTC TCTCTCTCTC TCTCTCTCTC TCTCTCTCTC TACATCATCG TTTGGGGGAT
  121 GAATCAAATG GGGTTGGCAA TTATCAAGGA ATGAATGGTT TTTGTTCACC CTCGCTTTAT
  181 TAGTCTTTCT CTCTACGCTG TGTTTGGTGC GTTTGCCTTA AACCACACTC GGTGTATTAG
  241 GGGTTGGCAA CTTATCATAG CTTCGTTCCT CATGCATGCA TGTATGGTTC ATCATGITTT
  301 TGTCAAATTT TCATGTAGCA ACATATTGTC CTCCGTCCAC AACAGATAAG CTGATCCTGC
  361 TAGTCATAGC TGCTATATAC AGATCAGCTT ATTAAGTTTG CATCATTGTA GAAGCAAAAG
  421 TCATGTAGCA CCCGGGCGGC AGACATGTTA CGTACGTATA TAACAGGTTG TTGTTATGCG
  481 TGTICTAATG TTCCTTGGCA CAACAACTGT AGTGATACAT GCAGAGGGAG CGGAGGAGGA
  541 GGAGATAGAA ACCAAAGGGA GGAGGACGCG GCGGCGGCGG CGGCGGCAGA GGCCGGCTAC
  601 GGCAGGCAGC TGGTGATTCC CGAGGACGGG TACGAGTGGA AGAAGTACGG CCAGAAGTTC
  661 ATCAAGAACA TCCAGAAAAT CAGGTACTTG CTCCGTTCGA TCCAACATAT GCATACGTAG
  721 CATTTTTGGC ATCGAGATTG ATCTCGAGCT CTCAAATAAA GCTAGTGCAA ACTTGATCAC
  781 ATATACCATT TTTTCGTGGT CAAATCTCGT TTCCCGCCAT ACGCGTGTAC ATCAGATTAA
  841 TCAATAGCTC GACGTTGACC AAGCTTGTTG ACTIGTTCAT CTTCGTTCCT GTGCATCAAA
  901 TCGTTTTATT AATTAATTGA GTCGATGTGA CGCCCATCGA TCGATCACTG GTATAATGGA
  961 ATGTATGGGT TGCCCGCCGT CCCCGTGCAT ATATGCATAC GTGCAATGCT CTGCTGCCAG
1021 ATCTTATCTT TCGAAGAAGA ATCAACGGAA GAATAATATC CTCGCTTTAT TATATTATAT
1081 ATTGATAACG GTCGACCAAA TAAAGCCCTG ATGATGACTT GATGAGCAAA CTGCACAAGT
1141 GTGTTTTGCA TTGCATGCCA ACTGATGATA CCACCGTACG TGGTGATTCC ATGATGCATG
1201 TGTGTGATCA AAATCCAACA ATGGCGCAGG AGCTACTTCC GGTGTCGGCA CAAGCTGTGC
1261 GGCGCCAAGA AGAAGGTGGA GTGGCACCCG CGGGACCCCA GCGGCGACCT CCGCATCGTC
1321 TACGAGGGCG CGCACCAGCA CGGCGCCCCG GCGGCGGCGG CTCCTCCCGG TCCCGGCGGC
1381 CAGCATCAGG GCGGCGGCGC CTCCGACTTC AACAGATACG AGCTGGGCGC GCAGTACTTC
1441 GGCGGGGCCG GCCGGTCGCA TTGA
```

(SEQ ID NO:2, Sp01g012870, S. propinquum), or a variant thereof having at least 90%, 95%, or more sequence identity to SEQ ID NO:2.

In some embodiments, the coding sequence of the shattering Sh1 gene in S. propinquum, including introns and 5′ untranslated region, can have the nucleic acid sequence:

```
      1 TAAGATGACT CTATTTTTTA TCAATAAGCA CTTTGTACTA TGATTAAGAC AAAAGGAAGA
    61 GAGGGGACAA GAATTACAAA CTATACTTAG GGGTTGTTTG AATTTCAGTC ATAGTTGGTC
  121 ACAACTCAGA TGTGGTGAGA CACACTCTAT GATGAGAATA ATGAGATCTG TTTGGTTCTC
  181 TTCTCACCTA GGCTACATCG CATCTGGAGC GAGAGACAGG CTAGCCACAG CCTGAGATGG
  241 TGCATGCACC TGCACTTGTT TGGTTTTGCT CTTTGTTTTG AGCCACTCCA GCCATGTCTC
  301 GGAAAGATAT TGTTTTGTTG GTCTTTGGCT TGGCACCAGT GCTCTCTCAC GCGTACAGGC
  361 ACACGCTCTC TTTTGGCTCC ACGCAGCCAT GTGTTGGCTA AAAATGATTT TAGAATCCAT
  421 TTCCCATGAG CCTGAGATGG TTGCACGCAC TATAGGTCTA ACCCTGGTAG CACTTTAGGT
  481 AACCAAACAC CTTAAGCCTG CATCCCAAGA GCCAGGCCAG TTTGGAAACT GGACAACCAA
  541 ATAGGCCTCT AATGAATTTG ATGTGTTGTA TTCTGTGGGT GTCTAGCACT CTTCACCAAC
  601 TAAACACTGA TAAAAAAAAG TTATGGTGTG CGATGCCTTA GTGTTGGCTA GCAAGTGAAG
  661 GCCGGGAACC AAACATGCTT TTACTCTTTC ATATCTTAGG CCATGTTTGG TTTGTCGTAG
  721 TAAACTTTAA CTTCCATCAC ATCAAATATT TGAGCACATG CATAGAGTAC TAAATATATA
  781 GACTATTTAC AAAATTAAAA ACACAACTAG AGAATAATTT ATGAGACAAG TTTTCTGAGC
  841 CTAATTAGTC TATGATTGGA CACTAATTGT CAAATAAAAT AAAAATACTA TAATACCTAT
  901 TAAACTTTAA TACCTTCGAC CAAACAAGCC CTTACAGGGT TTCAAATATG TATATAAAAT
  961 TATTTTCGTT AAGCTTTCAT ATTAAACTTC TCATTGTTGT CTCATTACCA TCTTTCCCTG
1021 CAAAATGTGA AAACAAGGTG GATAAATACA TGAATCCACA TCTGTTCTCA CCCCTAGTAT
1081 TTAGTAAAAG GAAATAGTGT ACTCTCTCAA GTACAAATAA TAATGTTTCT TGACTTCAAC
1141 ACCTCTAACA CAAAATCGTA ACTAATATTA TTTGTGTAAT AATATATATC TATAAAAGAA
1201 CATGTTGCCT CTCTCTAGAA AAGTCTACCT CTTGATGTCA TTTTCCAAAT ATCAAAACTC
1261 GATACACAAA AGAATTGATT TAGAACCAAA GATTAAAATG CCTGACTACA TGATGAAACC
1321 TGAAAACATT GTTCTATTAT TAGTGACTGA AGGGAGTAAT ATCCAACAGT AACTTCTTGT
1381 TGCGAAGATT AGTGTTGTAC GCAAAAAGAA ATATCCATAT TCCTCCATAT AAAGGAGATG
1441 ATGAGATCAC AGTGATTTTC TGGTTCAGTC AAAACCAGTG GCAAAGTTGG GTAGGGAATT
1501 GAAGCATGTG AACCCAAAAA TTTACTGATT CGTCTTCGTC TTGACGACGT TAACGTCGTC
1561 GCATCTGAGA AACTTCCATT CGATTGACTA ATAAGCCCTG ATAATAAATA TACCACACCC
1621 AAAGAGCTTC ATCACTACTC TCTCAATCTC TCTCCCTCTC GTCTACATGG TTCATTCATT
1681 AAACTTTGCG ACAACATGGG AGCAGCAGTA GAGCACAGGA CGTCGTAGAC GTACGGTCAC
1741 TGGCGGCGTC CATGGATTCA AGCTCACAGC CCGGCGCAAT GTATGCATCT CTCTCTCTCT
1801 CTCTCTCTCT CTCTCTCTCT CTCTCTCTCT CTCTCTCTCT CTCTCTCTCT CTACATCATC
1861 GTTTGGGGGA TGAATCAAAT GGGGCTGCCA ATTATCAAGG AATGAATGGT TTTTGTTCAC
1921 CCTCCTTATA TTAGTCTTTC TCTCTACGCT GTGTTTGGTG CGTTTGCCTT AAACCACACT
1981 CGGTGTATTA GGGGTTGGCA ACTTATCATA GCTTTGGTTC TCATGCATGC ATGTATGGTT
2041 CATCATGTTT TTGTCAAATT TTCATGTAGC AACATATTGT CCTCCGTCCA CAACAGATAA
2101 GCTGATCCTG CTAGTCATAG CTGCTATATA CAGATCAGCT TATTAAGTTT GCATCATTGT
2161 AGAAGCAAAA GTAATTAAGC ACCCGGGCGG CAGACATGTT ACGTACGTAT ATAACAGGTT
2221 GTTGTTATGC GTGTTCTAAT GTTCCTTGGC ACAACAACTG TAGTGATACA TGCAGAGGGA
2281 GCGGAGGAGG AGGAGATAGA AACCAAAGGG AGGAGGACGC GGCGGCGGCG GCGGCGGCAG
2341 AGGCCGGCTA CGGCAGGCAG CTGGTGATTC CCGAGGACGG GTACGAGTGG AAGAAGTACG
2401 GCCAGAAGTT CATCAAGAAC ATCCAGAAAA TCAGGTACTT GCTCCGTTCG ATCCAACATA
2461 TGCATACGTA GCATTTTTGG CATCGAGATT GATCTCGAGC TCTCAAATAA AGCTAGTGCA
2521 AACTTGATCA CATATACCAT TTTTTCGTGG TCAAATCTCG TTTCCCGCCA TACGCGTGTA
2581 CATCAGATTA ATCAATAGCT CGACGTTGAC CAAGCTTGTT GACTTGTTCA TCTTCGTTCC
2641 TGTGCATCAA ATCGTTTTAT TAATTAATTG AGTCGATGTG ACGCCCATCG ATCGATCACT
2701 GGTATAATGG AATGTATGGG TTGCCCGCCG TCCCCGTGCA TATATGCATA CGTGCAATGC
2761 TCTGCTGCCA GATCTTATCT TTCGAAGAAG AATCAACGGA AGAATAATAT CCTCGCTTTA
2821 TTATATTATA TATTGATAAC GGTCGACCAA ATAAAGCCCT GATGATGACT TGATGAGCAA
2881 ACTGCACAAG TGTGTTTTGC ATTGCATGCC AACTGATGAT ACCACCGTAC GTGGGTGGTC
2941 CATGATGCAT GTGTGTGATC AAAATCCAAC AATGGCGCAG GAGCTACTTC CGGTGTCGGC
3001 ACAAGCTGTG CGGCGCCAAG AAGAAGGTGG AGTGGCACCC GCGGGACCCC AGCGGCGACC
3061 TCCGCATCGT CTACGAGGGC GCGCACCAGC ACGGCGCCCC GGCGGCGGCG GCTCCTCCCG
3121 GTCCCGGCGG CCAGCATCAG GGCGGCGGCG CCTCCGACTT CAACAGATAC GAGCTGGGCG
3181 CGCAGTACTT CGGCGGGGCC GGCCGGTCGC ATTGA
```

(SEQ ID NO:3, Sp01g012870, S. propinquum), or a variant thereof having at least 90%, 95%, or more sequence identity to SEQ ID NO:3.

In some embodiments, the coding sequence of the shattering Sh1 gene in S. propinquum, including introns and 5′ untranslated region and 3′ untranslated region can have the nucleic acid sequence:

```
      1 TAAGATGACT CTATTTTTTA TCAATAAGCA CTTTGTACTA TGATTAAGAC AAAAGGAAGA
    61 GAGGGGACAA GAATTACAAA CTATACTTAG GGGTTGTTTG AATTTCAGTC ATAGTTGGTC
  121 ACAACTCAGA TGTGGTGAGA CACACTCTAT GATGAGAATA ATGAGATCTG TTTGGTTCTC
  181 TTCTCACCTA GGCTACATCG CATCTGGAGC GAGAGACAGG CTAGCCACAG CCTGGTCTGG
  241 TGCATGCACC TGCACTTGTT TGGTTTTGCT CTTTGTTTTG AGCCACTCCA GCCATGTCTC
  301 GGAAAGATAT TGTTTTGTTG GTCTTTGGCT TGGCACCAGT GCTCTCTCAC GCGTACAGGC
  361 ACACGCTCTC TTTTGGCTCC ACGCAGCCAT GTGTTGGCTA AAAATGATTT TAGAATCCAT
  421 TTCCCATGAG CCTGAGATGG TTGCACGCAC TATAGGTCTA ACCCTGGTAG CACTTTAGGT
  481 AACCAAACAC CTTAAGCCTG CATCCCAAGA GCCAGGCCAG TTTGGAAACT GGACAACCAA
  541 ATAGGCCTCT AATGAATTTG ATGTGTTGTA TTCTGTGGGT GTCTAGCACT CTTCACCAAC
  601 TAAACACTGA TAAAAAAAAG TTATGGTGTG CGATGCCTTA GTGTTGGCTA GCAAGTGAAG
  661 GCCGGGAACC AAACATGCTT TTACTCTTTC ATATCTTAGG CCATGTTTGG TTTGTCGTAG
  721 TAAACTTTAA CTTCCATCAC ATCAAATATT TGAGCACATG CATAGAGTAC TAAATATATA
  781 GACTATTTAC AAAATTAAAA ACACAACTAG AGAATAATTT ATGAGACAAG TTTTCTGAGC
  841 CTAATTAGTC TATGATTGGA CACTAATTGT CAAATAAAAT AAAAATACTA TAATACCTAT
  901 TAAACTTTAA TACCTTCGAC CAAACAAGCC CTTACAGGGT TTCAAATATG TATATAAAAT
  961 TATTTTCGTT AAGCTTTCAT ATTAAACTTC TCATTGTTGT CTCATTACCA TCTTTCCCTG
1021 CAAAATGTGA AAACAAGGTG GATAAATACA TGAATCCACA TCTGTTCTCA CCCCTAGTAT
1081 TTAGTAAAAG GAAATAGTGT ACTCTCTCAA GTACAAATAA TAATGTTTCT TGACTTCAAC
1141 ACCTCTAACA CAAAATCGTA ACTAATATTA TTTGTGTAAT AATATATATC TATAAAAGAA
1201 CATGTTGCCT CTCTCTAGAA AAGTCTACCT CTTGATGTCA TTTTCCAAAT ATCAAAACTC
1261 GATACACAAA AGAATTGATT TAGAACCAAA GATTAAAATG CCTGACTACA TGATGAAACC
1321 TGAAAACATT GTTCTATTAT TAGTGACTGA AGGGAGTAAT ATCCAACAGT AACTTCTTGT
1381 TGCGAAGATT AGTGTTGTAC GCAAAAAGAA ATATCCATAT TCCTCCATAT AAAGGAGATG
1441 ATGAGATCAC AGTGATTTTC TGGTTCAGTC AAAACCAGTG GCAAAGTTGG GTAGGGAATT
1501 GAAGCATGTG AACCCAAAAA TTTACTGATT CGTCTTCGTC TTGACGACGT TAACGTCGTC
1561 GCATCTGAGA AACTTCCATT CGATTGACTA ATAAGCCCTG ATAATAAATA TACCACACCC
1621 AAAGAGCTTC ATCACTACTC TCTCAATCTC TCTCCCTCTC GTCTACATGG TTCATTCATT
1681 AAACTTTGCG ACAACATGGG AGCAGCAGTA GAGCACAGGA CGTCGTAGAC GTACGGTCAC
1741 TGGCGGCGTC CATGGATTCA AGCTCACAGC CCGGCGCAAT GTATGCATCT CTCTCTCTCT
1801 CTCTCTCTCT CTCTCTCTCT CTCTCTCTCT CTCTCTCTCT CTCTCTCTCT CTACATCATC
1861 GTTTGGGGGA TGAATCAAAT GGGGCTGCCA ATTATCAAGG AATGAATGGT TTTTGTTCAC
1921 CCTCCTTATA TTAGTCTTTC TCTCTACGCT GTGTTTGGTG CGTTTGCCTT AAACCACACT
1981 CGGTGTATTA GGGGTTGGCA ACTTATCATA GCTTTGGTTC TCATGCATGC ATGTATGGTT
2041 CATCATGTTT TTGTCAAATT TTCATGTAGC AACATATTGT CCTCCGTCCA CAACAGATAA
2101 GCTGATCCTG CTAGTCATAG CTGCTATATA CAGATCAGCT TATTAAGTTT GCATCATTGT
2161 AGAAGCAAAA GTAATTAAGC ACCCGGGCGG CAGACATGTT ACGTACGTAT ATAACAGGTT
2221 GTTGTTATGC GTGTTCTAAT GTTCCTTGGC ACAACAACTG TAGTGATACA TGCAGAGGGA
2281 GCGGAGGAGG AGGAGATAGA AACCAAAGGG AGGAGGACGC GGCGGCGGCG GCGGCGGCAG
2341 AGGCCGGCTA CGGCAGGCAG CTGGTGATTC CCGAGGACGG GTACGAGTGG AAGAAGTACG
2401 GCCAGAAGTT CATCAAGAAC ATCCAGAAAA TCAGGTACTT GCTCCGTTCG ATCCAACATA
2461 TGCATACGTA GCATTTTTGG CATCGAGATT GATCTCGAGC TCTCAAATAA AGCTAGTGCA
2521 AACTTGATCA CATATACCAT TTTTTCGTGG TCAAATCTCG TTTCCCGCCA TACGCGTGTA
2581 CATCAGATTA ATCAATAGCT CGACGTTGAC CAAGCTTGTT GACTTGTTCA TCTTCGTTCC
2641 TGTGCATCAA ATCGTTTTAT TAATTAATTG AGTCGATGTG ACGCCCATCG ATCGATCACT
2701 GGTATAATGG AATGTATGGG TTGCCCGCCG TCCCCGTGCA TATATGCATA CGTGCAATGC
2761 TCTGCTGCCA GATCTTATCT TTCGAAGAAG AATCAACGGA AGAATAATAT CCTCGCTTTA
2821 TTATATTATA TATTGATAAC GGTCGACCAA ATAAAGCCCT GATGATGACT TGATGAGCAA
2881 ACTGCACAAG TGTGTTTTGC ATTGCATGCC AACTGATGAT ACCACCGTAC GTGGGTGGTC
2941 CATGATGCAT GTGTGTGATC AAAATCCAAC AATGGCGCAG GAGCTACTTC CGGTGTCGGC
3001 ACAAGCTGTG CGGCGCCAAG AAGAAGGTGG AGTGGCACCC GCGGGACCCC AGCGGCGACC
3061 TCCGCATCGT CTACGAGGGC GCGCACCAGC ACGGCGCCCC GGCGGCGGCG GCTCCTCCCG
3121 GTCCCGGCGG CCAGCATCAG GGCGGCGGCG CCTCCGACTT CAACAGATAC GAGCTGGGCG
3181 CGCAGTACTT CGGCGGGGCC GGCCGGTCGC ATTGACGCGG GGCGCTAGTT CCTAAAATAT
3241 TTTGTAAAAT TTTTCACATT CTCGTCACAT CAAATTTTGC GGCACATATA TATATATATA
3301 GAGTACTAAA TATATATAAA AAAATAACTA ATTACATAGT TTACCTATAA TTTATGAGAC
3361 GAATCTTTTG ATCCTAGTTA GTCAATAATT AACAATATTT GTTAAATACA AACAAAATTA
3421 TTACTATTCC TATTTTA
```

(SEQ ID NO:4, Sp01g012870 transgene, S. propinquum), or a variant thereof having at least 90% sequence identity to SEQ ID NO:4.

In some embodiments, the coding sequence (without introns) of the candidate gene Sp01g012880 as it is found in S. propinquum, includes the nucleic acid sequence:

```
    1 ATGGCGGAGC CGGGGCTCGA GGGCAGCCAG CCGGTGGATC TGTCCAAGCA CCCCTCCGGC
  61 ATCGTCCCCA CGCTCCAGAA TATTGTATCA ACAGTTAATT TGGATTGTAA ACTTGACCTC
121 AAAGCAATAG CTTTGCAAGC ACGAAATGCG GAGTATAACC CAAAGCGTTT TGCTGCAGTC
181 ATCATGAGAA TAAGGGAACC CAAAACCACA GCACTGATAT TTGCATCGGG TAAAATGGTA
241 TGTACTGGAG CAAAGAGTGA ACAGCAATCT AAGCTTGCAG CAAGAAAGTA TGCTCGTATC
301 ATTCAGAAAC TAGGTTTTCC TGCTAAATTT AAGGACTTTA AGATTCAGAA TATTGTTGGC
361 TCTTGTGATG TCAAGTTTCC AATTAGGCTT GAGGGCCTTG CATATTCTCA TGGTGCCTTC
421 TCAAGTTACG AACCAGAACT CTTTCCTGGC CTTATCTATC GGATGAAACA ACCAAAGATT
481 GTTCTTTTAA TTTTTGTTTC AGGCAAGATT GTTTTGACTG GAGCAAAGGT GAGAGAGGAG
541 ACTTACACTG CCTTCGAGAA CATCTATCCT GTACTGACAG AGTTTAGAAA AGTTCAGCAA
```

(SEQ ID NO:5, Sp01g012880, S. propinquum), or a variant thereof having at least 90%, 95%, or more sequence identity to SEQ ID NO:5.

In some embodiments, the region between two SNPs that show high levels of genetic association with the shattering trait, including both Sp01g012870 and Sp01g012880 in S. propinquum, has the nucleic acid sequence:

```
        1 GTCCTTCTTC CTCCGGCACC CATAATAAAC AAAACAAACT ACACGATCGA GATCTCGCCA
      61 GGATTTAATT TGACACGTGC ATGGATCACG TACGGTTTGT TGGATCGTCT CCAACAATAA
    121 GACGAATGAA CTGATAGTAC TATATACGCC TACACCCACC AACGTGCATG GATCACACGG
    181 TTCAATTAGT TTGTCTTCCA CACGTGCATG GAACCGTGAG TCATTCAGAA TCGTAGCCTT
    241 AATTTGATCA ACCAGTATGT CCATCCGTTA AAATGCTCCA CTAAACATAT ATTAATATTT
    301 AAGAAGGTCG GAGTTCACAT TCACATGGAG ACTACTACTC GGAGACTACT ACTCGCTCTG
    361 TTTTGTTTTT GTAAGAGGGT GTTTGGGACT GCTCTGCTCC ATGTTTTCCA GCTCCGCTCC
    421 ATGTTTTTTA GCCAAACGGT TTCAGCTTCA TGCACTCAAG GAAAAAGGGT GGAGTTGTGA
    481 GAGCACCTAA AGAGGTACTC CACAAACTCC AGTTTTTTTT GGAGCTGCTC CATGGTAGAG
    541 TTTGTAAAGC AGAGTTTGTG GAGCAGTCCC AAACACCTTG ACGAAAGTTT TCAAGAAATC
    601 CAAAAAGTTT TCAAGATTTT TTGTCATATC GAATTTTGTG GCACATGCAT GAAGCATTAA
    661 ATATAGACGA AAATAAAAAC TAATCACACA GTTTGACTGT AAATCGTGAG ACGAATCTTT
    721 TGACCCTAGT TAGTCTATGA TTAGAAAATA TTTACCACAA ACAAACGAAA GTGCTACAGT
    781 AGCGAAATAT AAAAATTTTC ACTTCTAAAC AAGGCCCAGC TAGCGCTGGC TAAAGGGTAA
    841 AAGAAAAGAG GCAGCAGCTT CTTGGAACAA GACCACGCAA CGAGGGAACG GTTGCTGACG
    901 TAAGACAAGT GACGTCAGTC ACGGCTCCAG CCGCGACCTG GCGCGACATT CCCTCCTCTC
    961 CAAACCACGC GGCCCCCGCC CCGCTAACGG CCGTCCAAGG TTTAGGACGA TCGCAGAGCG
  1021 TGCTTTCAGG TTTGAATTTG ATCGGCATAA AGTTTCCGTT TGCTTGAAAT TTGTATATTC
  1081 GTCCTTATAA AATTGGTGTA TTATGGCCTT GTTTAGTTCC TAAAATTTTT TAAGATTTAC
  1141 CGTGACATCA AATTTTGTGG TATATGCATA GAACATTAAA TATAGATAAA ATGAAAAACT
  1201 AATTGTATAG TTTATCTGTA ATTTGCAAAA CGAATCTTTT AAGCCTGGTT AGTCCATGGT
  1261 TGAATAATAA TTACCAAATG CAAACGAAAA TGCTACAGTA GTAAAATCAA AAAAAAACAA
  1321 ACTAAACAAG GCACATGCAT GAAAGCTGAG AAGCGGATCG TTGGATTCTA CTTCTTTTGT
  1381 TTCAATTAGT ATGTTGTTTT AATTTTCCCT CCAGGAGAAG CAAACAAGTC ATTTGTTTGT
  1441 TTCAGCTTGC ATATTGTAAC AACTTATAAG ATGACTCTAT TTTTTATCAA TAAGCACTTT
  1501 GTACTATGAT TAAGACAAAA GGAAGAGAGG GGACAAGAAT TACAAACTAT ACTTAGGGGT
  1561 TGTTTGAATT TCAGTCATAG TTGGTCACAA CTCAGATGTG GTGAGACACA CTCTATGATG
  1621 AGAATAATGA GATCTGTTTG GTTCTCTTCT CACCTAGGCT ACATCGCATC TGGAGCGAGA
  1681 GACAGGCTAG CCGCGACCTG GTCTGGTGCA TGCACCTGCA CTTGTTTGGT TTTGCTCTTT
  1741 GTTTTGAGCC ACTCCAGCCA TGTCTCGGAA AGATATTGTT TTGTTGGTCT TTGGCTTGGC
  1801 ACCAGTGCTC TCTCACGCGT ACAGGCACAC GCTCTCTTTT GGCTCCACGC AGCCATGTGT
  1861 TGGCTAAAAA TGATTTTAGA ATCCATTTCC CATGAGCCTG AGATGGTTGC ACGCACTATA
  1921 GGTCTAACCC TGGTAGCACT TTAGGTAACC AAACACCTTA AGCCTGCATC CCAAGAGCCA
  1981 GGCCAGTTTG GAAACTGGAC AACCAAATAG GCCTCTAATG AATTTGATGT GTTGTATTCT
  2041 GTGGGTGTCT AGCACTCTTC ACCAACTAAA CACTGATAAA AAAAAGTTAT GGTGTGCGAT
  2101 GCCTTAGTGT GGCATAGCAA GTGAAGGCCG GGAACCAAAC ATGCTTTTAC TCTTTCATAT
  2161 CTTAGGCCAT GTTTGGTTTG TCGTAGTAAA CTTTAACTTC CATCACATCA AATATTTGAG
  2221 CACATGCATA GAGTACTAAA TATATAGACT ATTTACAAAA TTAAAAACAC AACTAGAGAA
  2281 TAATTTATGA GACAAGTTTT CTGAGCCTAA TTAGTCTATG ATTGGACACT AATTGTCAAA
  2341 TAAAATAAAA ATACTATAAT ACCTATTAAA CTTTAATACC TTCGACCAAA CAAGCCCTTA
  2401 CAGGGTTTCA AATTGGTGTA TAAAATTATT TTCGTTAAGC TTTCATATTA AACTTCTCAT
  2461 TGTTGTCTCA TTACCATCTT TCCCTGCAAA ATGTGAAAAC AAGGTGGATA AATACATGAA
  2521 TCCACATCTG TTCTCACCCC TAGTATTTAG TAAAAGGAAA TAGTGTACTC TCTCAAGTAC
  2581 AAATAATAAT GTTTCTTGAC TTCAACACCT CTAACACAAA ATCGTAACTA ATATTATTTG
  2641 TGTAATAATA TATATGCATA AAAGAACATG TTGCCTCTCT CTAGAAAAGT CTACCTCTTG
  2701 ATGTCATTTT CCAAATATCA AAACTCGATA CACAAAAGAA TTGATTTAGA ACCAAAGATT
  2761 AAAATGCCTG ACTACATGAT GAAACCTGAA AACATTGTTC TTCAATTAGT GACTGAAGGG
  2821 AGTAATATCC AACAGTAACT TCTTGTTGCG AAGATTAGTG TTGTACGCAA AAAGAAATAT
  2881 CCATATTCCT CCATATAAAG GAGATGATGA GATCACAGTG ATTTTCTGGT TCAGTCAAAA
  2941 CCAGTGGCAA AGTTGGGTAG GGAATTGAAG CATGTGAACC CAAAAATTTA CTGATTCGTC
  3001 TTCGTCTTGA CGACGTTAAC GTCGTCGCAT CTGAGAAACT TCCATTCGAT TGACTAATAA
  3061 GCCCTGATAA TAAATATACC ACACCCAAAG AGCTTCATCA CTACTCTCTC AATCTCTCTC
  3121 CCTCTCGTCT ACATGGTTCA TTCATTAAAC TTTGCGACAA CATGGGAGCA GCAGTAGAGC
  3181 ACAGGACGTC GTAGACGTAC GGTCACTGGC GGCGTCCATG GATTCAAGCT CACAGCCCGG
  3241 CGCAATGTAT GCATCTCTCT CTCTCTCTCT CTCTCTCTCT CTCTCTCTCT CTCTCTCTCT
  3301 CTCTCTCTCT CTCTCTCTAC ATCATCGTTT GGGGGATGAA TCAAATGGGG CTGCCAATTA
  3361 TCAAGGAATG AATGGTTTTT GTTCACCCTC CTTATATTAG TCTTTCTCTC TACGCTGTGT
  3421 TTGGTGCGTT TGCCTTAAAC CACACTCGGT GTATTAGGGG TTGGCAACTT ATCATAGCTT
  3481 TGGTTCTCAT GCATGCATGT ATGGTTCATC ATGTTTTTGT CAAATTTTCA TGTAGCAACA
  3541 TATTGTCCTC CGTCCACAAC AGATAAGCTG ATCCTGCTAG TCATAGCTGC TATATACAGA
  3601 TCAGCTTATT AAGTTTGCAT CATTGTAGAA GCAAAAGTAA TTAAGCACCC GGGCGGCAGA
  3661 CATGTTACGT ACGTATATAA CAGGTTGTTG TTATGCGTGT TCTAATGTTC CTTGGCACAA
  3721 CAACTGTAGT GATACATGCA GAGGGAGCGG AGGAGGAGGA GATAGAAACC AAAGGGAGGA
  3781 GGACGCGGCG GCGGCGGCGG CGGCAGAGGC CGGCTACGGC AGGCAGCTGG TGATTCCCGA
  3841 GGACGGGTAC GAGTGGAAGA AGTACGGCCA GAAGTTCATC AAGAACATCC AGAAAATCAG
  3901 GTACTTGCTC CGTTCGATCC AACATATGCA TACGTAGCAT TTTTGGCATC GAGATTGATC
  3961 TCGAGCTCTC AAATAAAGCT AGTGCAAACT TGATCACATA TACCATTTTT TCGTGGTCAA
  4021 ATCTCGTTTC CCGCCATACG CGTGTACATC AGATTAATCA ATAGCTCGAC GTTGACCAAG
  4081 CTTGTTGACT TGTTCATCTT CGTTCCTGTG CATCAAATCG TTTTATTAAT TAATTGAGTC
  4141 GATGTGACGC CCATCGATCG ATCACTGGTA TAATGGAATG TATGGGTTGC CCGCCGTCCC
  4201 CGTGCATATA TGCATACGTG CAATGCTCTG CTGCCAGATC TTATCTTTCG AAGAAGAATC
  4261 AACGGAAGAA TAATATCCTC GCTTTATTAT ATTATATATT GATAACGGTC GACCAAATAA
  4321 AGCCCTGATG ATGACTTGAT GAGCAAACTG CACAAGTGTG TTTTGCATTG CATGCCAACT
  4381 GATGATACCA CCGTACGTGG GTGGTCCATG ATGCATGTGT GTGATCAAAA TCCAACAATG
  4441 GCGCAGGAGC TACTTCCGGT GTCGGCACAA GCTGTGCGGC GCCAAGAAGA AGGTGGAGTG
  4501 GCACCCGCGG GACCCCAGCG GCGACCTCCG CATCGTCTAC GAGGGCGCGC ACCAGCACGG
  4561 CGCCCCGGCG GCGGCGGCTC CTCCCGGTCC CGGCGGCCAG CATCAGGGCG GCGGCGCCTC
  4621 CGACTTCAAC AGATACGAGC TGGGCGCGCA GTACTTCGGC GGGGCCGGCC GGTCGCATTG
  4681 ACGCGGGGCG CTAGTTCCTA AAATATTTTG TAAAATTTTT CACATTCTCG TCACATCAAA
  4741 TTTTGCGGCA CATATATATA TATATAGAGT ACTAAATATA TATAAAAAAA TAACTAATTA
  4801 CATAGTTTAC CTATAATTTA TGAGACGAAT CTTTTGATCC TAGTTAGTCA ATAATTAACA
  4861 ATATTTGTTA AATACAAACA AAATTATTAC TATTCCTATT TTATAAAAAA AAAATTCAAG
  4921 TAAACAAGGC CTAGGTTGAC AAACCGACAA GAAAGGCCGG CGGCGTTGCG TCACGTACGC
  4981 ATGCATCAGC TCCTGTACGT GCTGGCCTCT GCTGGCTGCC GCTGCATCGA TCGATCGCTT
  5041 TCGCTGCGCA CCGGAGGGCA ACGGCAGGTG CTGCCGGTGC CGGTTGACGC CTTGCGCCGG
  5101 CGCAACATGA TGTTGAGTGC GGACTAATTG TTGCTGCTCC GGTTAACTCT CTGGTCTAGT
  5161 TCTAGTGTAC GGTACTATTA GGACGATGGT GCATAATTGT AATTTTCATA TTGTATATGG
  5221 ATAAAAAAAT ATTTAGCTGA AAGTGGAAAC TAGCACCGTC GCTATTATGT TTTGTTTTTT
  5281 GCAACTCTAA AGTGTAAACT TGTGCTCTAG TAGTCGAAAG TCTCCAGAGT TGGGTTCGAG
  5341 GCCCTGGTCA CCCGGGCTTA CATTGCATCG CCTCTGAACT GAATGCGACA CTCGAGACCT
  5401 AGCTTTATCA GTGGGATAAA CCTAATTCGT TTAGTCAGCT TTAACATTCA ATCATTTGTA
  5461 GATAGCAAGG CATCAATGGG TAACGAACGC CGCACTGTAT CCCCTAACCT CTGCCGACAA
  5521 CTGATCACTG CAACGGCTGG GCATCCATTA CCAACAAGTT GGCAACATTA ATAAAATGTT
  5581 TTCGATTGAG GAAAACGGCA AACACAGTTC CATGCGATAC AAGACAGCTC GTTCGCCGAG
  5641 CAATCTTTCC AGATACGTTA ATAGGCATTC TTATACAGTG CGTAGAATTC AAATTATTCA
  5701 TCCTAGCATG CAACATCGAA AAAGTAAAAG AACCAAGTGC AGGTACATTT GGATACAGAA
  5761 ACAAGTCTAC TGCGTGGTCG ACTGACCGGT TCCTCCATAC AGTGATAACC AACAAGATTA
  5821 TTCCCGGTGT CCTCTACGAT ACAGCATCTC AAATACAACA GATAACTTAC AACCAGTCAC
  5881 ACAGTCCCGT CAGTAGTCAG TACATTGCCC CAGTTACCTA CAGTGCCAGC CTTTTCATCA
  5941 TCGCACAGCA CTGAAAGATA CTCAGAAAAG ACTTTAATAG ACTCGTGTCT CAAAGACAAA
  6001 GTAGGGCAAA ATTTATCTAC TCTTGTTAGC ACTCAAGTTA ACCACATGGG ACACAAACTA
  6061 CTCAAACTGA AGCATGATAG GTGTCCGTGT TCACCAGGGC CTACCCAAAT GGACAAATCT
  6121 GACAAGTCCA TCAGCTACCA CAACAAACCC ACCCAACCAT GACACACCGA GGCTCACAGA
  6181 AATTACAGGA TGCTATAAGT TCCGCCAGAC TTTTTATGTA CAGTTAGAAT TTATGGTCAC
  6241 ACAAAAAACC TCAAGGATGC TTGTAATTAG AAGAACGTGA CCTTCACTTG GGTCATCTGC
  6301 AAAGAGGGAA CCAGAAGGAA AAGATTAGTT TTAAATAGTT AATTCTAGTA CTGCACACAC
  6361 CGACACGAGT TATAAACAAT ATAAACAATC CATTTGGAAT ACAGAAATTT CACAGAAATC
  6421 ATGTACAATT CCAAGGGAAT CGGTCCATTT TCACAGGAAA ACACAGGAAA CAGGGGGATC
  6481 CCACATTCCA AAAGGGGCTT AACGAGAGAA GGAATTATCC CCTCAGGCAG CTATTTACAT
  6541 GCCATGACAT CTGATTTGAA TAACTAGAAT ACCATAATAA AAGTTTGTTT CGAAAACACA
  6601 GTAGAAAACA TGGTTCCAAC ATTTTACTAT CAAGTCTAAC AACAAATAAC ATATAGGTGC
  6661 CCAGTCCCAC ACATGTTCCA AAATGAGTAC AAGACATAGT GAACATAGTC AACAGAACAA
  6721 GAGAATCTCA ATTGTAGGAA GAGTCATGCA TGCACTACTG AAGCATGATA AAAAGAACTA
  6781 CATACCATTG CTGAACTTTT CTAAACTCTG TCAGTACAGG ATAGATGTTC TCGAAGGCAG
  6841 TGTAAGTCTC CTCTCTCACC TACAAACAAT CGACTATGAA ATGAAGGAGA AAGATAAGCA
  6901 AATCGCAGTA TAATTAAGCA TGAGCACGAA ATGACAACTA ACCTTTGCTC CAGTCAAAAC
  6961 AATCTTGCCT GAAACAAAAA TTAAAAGAAC AATCTTTGGT TGTTTCATCC GATAGATAAG
  7021 GCCAGGAAAG AGTTCTGGTT CGTACTGTAA AACAAATTAA AAATGTCATT ATCCAAAGAA
  7081 TGCAGACAAA AAAGGGTAAA AGAATTACTG TGATGTTAAA ATAAGCCATA ATTGGACATA
  7141 CACTTGAGAA GGCACCATGA GAATATGCAA GGCCCTCAAG CCTAATTGGA AACTTGACAT
  7201 CACAAGAGCC AACAATATTC TGAATCTTAA AGTCCTGGCA TAGAACAGTA ACTTAGCAAC
  7261 TGATGTACAA ATTGTTCAAA GTACAGGTCA ATGTACACAA GTATGAAAAT AGTTACCTTA
  7321 AATTTAGCAG GAAAACCTAG TTTCTGAATG ATACGAGCAT ACTGGAAATA CAGACAGGGG
  7381 TTAGAATTCC AAAGCCTCTC AGTAAACTAG ATCCAACTTA AATAAAATGG TAGCAAGCCA
  7441 TATGGCACCT TTCTTGCTGC AAGCTTAGAT TGCTGTTCAC TCTTTGCTCC AGTACATACC
  7501 TGGTCATAGA AAATTATCGG TTGCTTGCTT CAGCACTAGA ACACTTATGA TGGATTGATA
  7561 CAAAATTGTA GTTCTATATG AAAGAAATGC AGTTCTAGTA AACTTTCTTC ATTTGGAAGA
  7621 AAAGTATTTG ACACATCAAT ACATTTAATT AATATTGAAT ATGACAACCA AGAAACTCTA
  7681 CAATACTGAA CATTGATCCA AATAAAATCC CAAGTAAAAA ACCCACCGAC ATATATCATC
  7741 TGGTAAGGGA AAAATAGATT TGCCTAGGGT AGGCTAGAGA GGGTAAGAAC TTTATTCTCC
  7801 AATATTTGAT GATTGAGAGA GGTAGATTAG GACACAGAAA AACAAAAAGA TTAGCCTTTC
  7861 TATCTTTTGA CAGCACAGCA CCAAGGCAAC AAAACATGTC AAAAAAAAAA GATCAAATCT
  7921 GTTTACATAA AAAACATGCA AAATCCTTGA AAATTGACAG TATAAGACAA AAGATGTTGA
  7981 TGACATACCA TTTTACCCGA TGCAAATATC AGTGCTGTGG TTTTGGGTTC CCTTATTCTC
  8041 ATGATGACTG CAGCAAAACG CTGTTTACAG ATAAAAAAGT CAAATACGAA ATATAATGAC
  8101 AGAAAACTTA GCAAAATTCA GGTTGCTACA CTGTATCATC ATAACTGAGA AAGATTGCAT
  8161 TCAATAGAAT GCCTAAAAGA GCAAACAAGT CATATATAAG CTAAAAATTT AGAACTTGTT
  8221 TGTCAAAGAA TATTGTGGTT ATTCACAGGA CAAGCAGGAT ATGAGCATCC ATCTGGTTAA
  8281 AAACTAACCG TGCGCATCTC ATATCCCAGG CCATCCATTA GTTATTAGCA CAAAGCTATT
  8341 TGAACTCATG GACAAGATTG TACATCATTA CAAAGGATCA ACATACTTTA TATATCCATA
  8401 AATCTTCCAC TAGATAAAAC CACCAGTAAA TACCGTGCAG CCATTGCTTT GAGGTAATCA
  8461 CTATACCTTT GGGTTATACT CCGCATTTCG TGCTTGCAAA GCTATTGCTT TGAGGTCAAG
  8521 TTTACAATCC AAATTAACTG TTGATACAAT ATTCCTGTCA TGAAAAAATG GCACGTCAAA
  8581 CAGACCATGA TCAAAGAACT GCAGTAAACA TGTGAATTTT GTTTTGTAAA TCCAACATAG
  8641 GGTTCTTATT ATAAGTTTTT AGCATTGAAG AGACACTACA AGATGATTTT CATTGTTCTT
  8701 TTTTTATATG ATAGTGTGTG CTATTAATTT CTTCTTCATG CCAATTTCCA ACATGTACAA
  8761 TCATAACAAA TTTAAGACTA ACATTCAAGA TAACCTACCC TATAATGGTT GGATCATAAA
  8821 ATCTTTGTAT CAATCAAAGT CATTTCAGGA CTCAATATGG CACTAATAAG CCCATAGCAC
  8881 TTAATAATGA AATCACCTGC AGAAAAATCT TACACCTAAA TCATACTAAA AATCTTCCAC
  8941 AAAAGCTAGT TAGGTTACTT CTGGTTTGGG GACGGAGTGG GATGGAATGG TCATGTCCCT
  9001 ATTTTTTGGA CGGGATTGAC CCAGACCTTG TTTGGTTGGA CGGATAGGTT CATTCCAATT
  9061 TTTGTTTGGT TCTAAGGATA TGGTGGGATG GAACCCGCTG GAGTTTTAAC TCCATTAGAC
  9121 ACAATAATCC ATGGCCGCAC CAGCCATTGT CTCTACACCT ATTCTTGTTG TCTTCTTCGG
  9181 GTGAGCAAAG CCTGATTCCC AAGATTTTGT ACCACAGTCA CTCAACATCT CACAGCTCCG
  9241 GTGCCCAACA GCTGGGCACT ACCACCGCCC AAGAGCTTGG CCAACCCATT CGCCCAAGAT
  9301 CTCATGCAGA GATCTTGGCA TTGCCACCAT CAGAGATGCT CAACCTGCCC CACCAGAGAT
  9361 CTCATGTGGC CAGAGGAGGT AATTGGACCC GCTCCTTCCC ATGCTGGAGC TCACCCCACT
  9421 CCTCTCATAT ATCGTCGGCG CTAACCCAGT GCGCTGCATA TTCTCCAAAC ATCTCCTCTC
  9481 CTCTGGTTGC CTTGAGCTTG GAGCTTCCAC ATGCCCGCGC CCCTCCTTTT GACCACGCTT
  9541 GCACCAGGCA ATGCAAAGAT GGCGTGCAAC ACGGTCCGCA AGGAATGGCT TCATCCACTC
  9601 GCTTCAAGGG GACCGAGCTG TCCAAGTATT TCAGGAATAT GCCACTGCAA AAATGACCCC
  9661 ATCCCTAGCT CCTCCCAACC AAACACTGCT GAAAAAGGAT TGGCCCATCC CGTCTGGAAC
  9721 GTCCCTCAAT CCAAACCAAT GCATTTAACC CTCCCCAGGG TATGAGATAT CGAAACCTCA
  9781 GTCCGTGAGG CTGACTGTTT ATCATATTAC ACAATTTATG CACCAACCAG TCAAAACATG
  9841 GAATGGAAAT ATGGTAAGAA GAGATTATGC TTGCTGCAAC TATTACGCCA AGATGACAAA
  9901 CTTCAATAAG GAAATAGATC TCCTCTCCAG TTTGGCCCTC TCTCGTTCTC CCAAGTTTCA
  9961 TACCTGAAAT CAACCCTCGG AGAGAGGATG ACAACTAAAT AATTCCCACC AAAGCCCCAA
10021 CTATTTAAGA CAATATTAGC TCGTTTCGAT GCACCCAGCA CTGGGAAGCT GAACAAAAAC
10081 ACGGCATAAA CCAACCACAC CACCACCCAC AAGACAGGGA GGCACCCCGC TGGCCAGAAC
10141 CAAGCCTTGG CAGCTCCACA GCACACCCAA GCACCCATCC GCCGGGCGGC GGGACCCTAG
10201 CACGTACGGT ACGGGATCTC TCCGGAACCC CGAATCCCCG ACGACCCAGA TCCGGGACTT
10261 ACTGGAGCGT GGGGACGATG CCGGAGGGGT GCTTGGACAG ATCCACCGGC TGGCTGCCCT
10321 CGAGCCCCGG CTCCGCCATC CGAACCACGC ACGCGACCTC GGCGGGGCTC CGCGCCGCGA
10381 ATCCGGGCTC AATCCGGGGC CGAAATGGGC GGGAAAGGAG CGCGCGCGTC ACCGGTTCGA
10441 GGGGGAATTC GAAATCCGGG TCTTTTATAG AGATCGGGAG AGGAGTTGGG GAGGAGGGAA
10501 AGCAAGGGGA AGGAGAGCTA GGGTTATCTG TCTCGCGAGG GGGAGTCGGG GACAGCGCGG
10561 GCGGCGTGAG AATGCGGGGG GAAGAGGGGG AGGTCGTCTG GTGGTGGGAG GTAGATGCGT
10621 GCGGGAGTTG GGGTTGTATC GGTGGACGGG GAGCAGGCGG TGGATGGCGA GTGCTTGGCT
10681 TTTGTAGGGG AACAGGGTGC ACCGGCTGTG GCCGGTTACC ACAGGGCGCG GTTTGCCCAC
10741 GCGCTGGTTC GAGTTATACA AACTGACCTG TGGGTCATAG CATGCGGTGG GGCCCGGTGT
10801 CGGTGTGTGG GTATGATGCG CGTTCGACGG CCATTAATCA AGAATTTCTC CTGCTCGCAA
10861 ATCGCACTAG CAGGTTACGA ACGCACCGAG AAGATCGTAC TATGGTTCTT TGAAAGAAAA
10921 TTATTATGAA TTATGAAATG ATGAATGATG AACTATACTA ATCGGACTGT TTGAATTATT
10981 GTGATGGATC ATTTTCGTTC GAGTGGGAAA TCATGGTCAC CAAAAAGCTG GTAAGAGAGA
11041 GAGATTATAT ATAATCGAGT GTTTTAGTTA TGTTTAGTTC ATAATTAACT TATTTTAGCT
11101 AATTATTATA ACCATAGTGG ATCCAAACAG GCCTGACTAG TGACTACTTG AGCATTCGCG
11161 TTACGTCACT GTTGCAGTGC ACATTCATTC GTATTAACTA AAACATCTTG CATTAGAGCT
11221 TCCCTGATGC ACCACGGTGG CGTGCTGTCG CAGTGACCAC CTTAGCTTTA GACTTCCATG
11281 TCATAGGAAG TTAAGCCTCG TAGAGTCTCA TGTTCTCTTG CAGAGAAGAT CATGGCCTCA
11341 TCTGACAAAA ATTAAAAGCA ACGGCTATGA ACAAGTATTA TAGTGAGCTG TAAGCTGACT
11401 AAATGCTGAG GTGGGGGAGA GAAGAAATGA GAGAGAAGAG AAGCAGGCTA TAAGGGCACT
11461 CACAATGCAA GACTCTATCA CAGAGTCCAA GACAATTTAT TACATATTAT TTATGGTATT
11521 TTGCTGATGT GGCAGCATAT TTATTGAAGA AAGATGTAGA AAAAATAAGA CTCCAAGTCT
11581 TATTTAGACT CTGAGTCCAC ATTGTTCGAG GTAATAAATA ACTTTAGACT CTATGATAGA
11641 GTCTGCATTG TGAGTGCCCT AAGCTTATAG CCAGCTTAAG CACAGGAACC AAGAAACTTT
11701 GTGAGAGATA AGTAGGCCAT ATATTAATAA TGAATAGTTA ACTATTGTAT GTGTGGGTTG
11761 GGAGAAGGCT GTAAAGAACC TTAGGGCACT CACAATGCAA GACTCTATCA CAGAGTCCAA
11821 AACAATTAAT TAGATATTAT TTATGGTATT TTGTTGATGT GGCAGCATAT TTATTGAAGA
11881 AAGAGGTAGA AAAAACAAGT CTCCAAGTCT TATTTAGACT CTAAGTTCAT ATTGTTCGAG
11941 ATAATAAATA ACTTTAGACT CTATGATAGA GTCTGCATTG TGAGTGCGCT TACACCAGCA
12001 AGTGGCCTGT ATTATTAAAC TTGCTCTAAG TAGCGCGATG TGGTGAGAAT AGTGACTCTA
12061 GGCTATTGGG ACCACGTCTG GTTCGTGCAT TTGGCTCCAA ATTGTCTCAG CGATTGACGG
12121 TCGGACCCCA GACAAGCCAC ATGCAGCTTT GCATTGAGTA AAAACGGTGG TTTTAACTTT
12181 TAATCCAACG GACGTACGTG GATGGTCACC TTTTTTCCTA GAGCTAACGC TACTAGGTGC
12241 CCGTGTTGCG ACGACTCCTC CACAATGGTG AACATCGATG TGTCAGTAAG CATGTCAGTG
12301 AGCATCGGTT CATAAGAGAG CTGCAATGTC TAAGCATCAT GTGGGACCAC CCAAATGAAT
12361 AAACAAACAA GGAGACATTG CAATGCCTAA ACATATCATT GAGCATTAGT TGAGACTCGA
12421 CCTCTCTCAC TATGTGCAAT AGTTTTTTTA TGTTGCACCG TGGAAAGTAG AAGCCTCGAT
12481 GCCGCGCAAA AAAAATTCAG CATCACACCC CAAATGTGAT GCCTCGAGGC GAGAAGCCAA
12541 AATATGTGCA TTGGTAAAAC TATACGTTAT GCGTAGTCTT ATATATAAAA TGTTAGCAAA
12601 AAATTCTTTC ATTTTAGAAT GGAGATAGTA GGCAATAAGA CCAGTACAAA ACGGACATAA
12661 ATCTAAAACA AATATTGTTT GAGAGAAAAG ATCTAAAATC AATCCAAGTA GAAGCAAGCA
12721 TCATATGTGA CATAATAAGA GATTAATAAT CCTAAAATGA GTGTACATGT CTTGCATCAA
12781 TTTATGAAAC TCGAATTATC TGTCTCCCAG AGCACAAGCC AATGCTACTC ATAACCTATT
12841 ACATATACGT CAATCTTTTA CAGAACTTGT GATCATCTTT ATATATGATC ATCATTTAAC
12901 GATCTGCGGG ACTAGTAGGC TATCAGAAGC AATAACCTTC GGTTGTTTCA GATGGACACG
12961 AATGTGCATC ACCAGTTTAC AGCTCTGTAT ACTTCACCTA ATAACTGAAC ATTCTGAGAG
13021 AATGAACTAT TTGTGGCTCC TTGATGAGGC CCAGCATGTT TACCTTTTAG GTTCCCTTAG
13081 GTTAAACACT AAATCTTCAT GATGGAAGGT GTTTGCCTGA ACTCCAAGAC AGCAAGGTTT
13141 TCTCTATACT TCTTTACTTC GGCCACCATT CTGTTGTACG ATTCAGGGTA TTTGCAAAAA
13201 ATCACGATTT TGATTCAGCT CCCTGGCTCG TGCCTGCAAT GTCAACATGA TCCTTTACAA
13261 ATGTTCGAAG GCATCCATTA ATTACCCGAG GGGCACCACC ATCACAAAAT CGCTTTGCCA
13321 GATCTACTGC CTGAAAGACA AGGGTCGAGA GACTTTTATT CTACTAGTAC TCAAAAATGG
13381 AAAGAGTAAT AGCTATAAGA AAACATGCAG GTGCTAGATG CATAAAGTCA AAATATGAAG
13441 AAAAACAAGT AATTGGGAGA AAATAAGCAC CTCATTAATG ACAACTTTGT GAGGTGTTCC
13501 TTTTGATGTC ATCTCTGCCA TAGCAATATG TAGAATGCAG AGCTCAAGTA TCCTTGCCAC
13561 AGGCTCATCC TGCCATGAAT TTTTCCATGT ATCAACAGCA GGTTATGCCA TAAAACAAGA
13621 CAGCAAAATA ATAAATACTA AAATATTTAA CCAGTTTAAA GATCAGGTAG ATTATAAACT
13681 GATGAAAGGA AAGTAATATA TTGTGTTTCA TATTTTTCTA ATTTTTACTT TAAAAAACAT
13741 CTGAGCTATG GTAGTAGAAA CAAATATAGA AATAAAGCGA TTCAGATTAA GGAAGGTGCA
13801 TTCTTCAGAT TCTGTATCAC TTCCTCATCC TTGGGGTGGC CAACAGAAAT AACTAATTAA
13861 CTATGCTGGA AAATTAAGTA GTGTAATAAG GCCATAAGTC TAAAATAACA ATGGGAGATC
13921 TCAATATTTC ACTGCATGCC AAAAGATAAG GCAGGAAATA ATCTTTGATG GTCACATGCT
13981 TTTGGTATGC ATCAGAGTGA TTGTTCACTA GTTCAGTGTA GTGAAAAACA GTTGTGTAAT
14041 ATACAGAATA AGGATACGTT CAAATCAAAC TGATAACCAT ATATAAACAT CTTCTGGTAT
14101 GCATTGTTCA CTAGTTCAGT GTAGTGAAAA ATGGTTGTGT AATATACAGA ATAAGTATAT
14161 GTTTGAACCA AACTGATAAA CATATAAACA GCTTGATGCA TATCGCAGGG ATTTGATGAA
14221 TCAACATAGA ATATTAGGAA AAGGTATCTA ACCTTCCAAG CCTGGGGAAT TATTTTGTCA
14281 ATGATATCTA CATGCTTATC CCATCCACTA GCAACAGCCA CTAAAAGTTC CCTGGACAAC
14341 CTGTACTTGA AAAGTATATA ATTAGGAATG TAAGAGCAGC AGGACTAAAT ATTGAACAGG
14401 AAATTAAATT TTATCATATA TCAGAACAGT GTATCGATAC CTAATGCCTT TAGTGGAATG
14461 GGGCAAGAAG GAAAGTATAC CGTAAGACGA AGTTGTTGTA CACCAGTTTT GGAGGAGCTG
14521 AAAGTACATC TTCTTCTGAA TATGAAAGAA AAACATGTCA AATTCTTTGC AGAAGAATAA
14581 CCAAACATTA ATGGAACATA TTTACACAAA AACAAATCTA TAGTTACTCA GCTGATTTCA
14641 CAACAGACTA AGGAAGAAAA TGTATATGGT TAATATGACT ATATGAGCTG TTTAGCACGC
14701 ATCGTAAGGA TACGTTTATT GTGCTGAACG AGATAGATGC CACTGGGCTG CTACAAAAGA
14761 TGCATGCTAA CGAAGGTGAA CAGTTTTCAG CATGTCGATT AAAAGTGTAA TCAATACATA
14821 GCTTGGTAAA ATATATCAAA ATTTACTGCC GCTTAGAGTG ATGGATTATG GTATAGCTCT
14881 CTTAAAACTC AGTCTGCAAC CCCCCCCCCC CCCCAAAAAA AAAAAAAAGA CACACAACCC
14941 CCTTAGATCT TGACGACCTA GCCTGACTAG GTAGCACCTA GGCATTAGCC ACTATACCGA
15001 ATCAAGAGTT AGGTGCCACG CAGCTGCTTA CCTAGCACAT TGCGTTTTTT TAAGCCAAAG
15061 CACTGCGTTA ACTGTTCTAG TTTGACGGTC TGAAATTCAC AGCACCAACT TGAAATTGCT
15121 CTAGCATGCC CTCCAGTTTT TATATACATG AAAATAGGCA CACGCCCACA ATAAAAAAAA
15181 AAGAAAATTG GCCTAAGTTC AATAATGTAT TTATGGAACA ACCAATGATC CATTGCTCTC
15241 TTTACTTTAG GAAATCAGAA TCATAGATAT ATGACATAAA GTTTCAAAAC TTAGACTGAA
15301 ACCCACCATA AAATTTATTT AAACAGGAAT CAACTAGATT TTCTGGTGGT TGTATGTTTC
15361 AGATTGACCG AAGGATAACC ATTAAAAGAC TGCTATAATG GAATTGGTAC CTAACTGAAC
15421 TTGTGCTCTT TGGAATCTTC TGGATATAGA GATATTCCAT CTCAAAATTG TGAAAAAAAG
15481 ATGGACATAT GTCCAATTTA CCAACAACAA TCTACTACTC CAGCTGTAAC AGCGTTAACA
15541 TATAGGAAGT AG
```

(SEQ ID NO:6, Sp01g012870 and Sp01g012880, S. propinquum), or a variant thereof having at least 90%, 95%, or more sequence identity to SEQ ID NO:6.

Accordingly, in some embodiments, a nucleic acid sequence containing the Sh1 gene as it is found in S. propinquum includes the nucleic acid sequence of SEQ ID NO:1, 2, 3, 4, 5, 6 or a fragment or variant thereof.

A polynucleotide is disclosed having a nucleic acid sequence SEQ ID NO: 1, 2, 3, 4, 5, 6, or a fragment or variant thereof. Also disclosed is a fragment or variant of the Sh1 gene as it is found in S. propinquum having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1, 2, 3, 4, 5, or 6. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more nucleotides shorter than SEQ ID NO: 1, 2, 3, 4, 5, or 6.

Also disclosed is a polynucleotide that hybridizes under stringent conditions to a polynucleotide consisting of the nucleic acid sequence SEQ ID NO: 1, 2, 3, 4, 5, 6 or a fragment or variant thereof

2. Non-Shattering Sh1 Gene Disclosed are polynucleotides having a non-shattering Sh1 (also referred to herein as sh1) gene from a sorghum plant. The Sorghum plant can be S. bicolor. Sequences for the non-shattering Sh1 gene in S. bicolor are provided.

In some embodiments, the non-shattering Sh1 can be overexpressed to inhibit endogenous Sh1 by acting as a competitive inhibitor.

In some embodiments, the coding sequence, without introns, of the non-shattering Sh1 gene as it is found in S. bicolor can include the nucleic acid sequence:

```
    1 ATGCCCGAGG ACGGGTACGA GTGGAAGAAG TACGGCCAGA AGTTCATCAA GAACATCCAG
  61 AAAATCAGGA GCTACTTCCG GTGTCGGCAC AAGCTGTGCG GCGCCAAGAA GAAGGTGGAG
121 TGGCACCCGC GGGACCCCAG CGGCGACCTC CGCATCGTCT ACGAGGGCGC GCACCAGCAC
181 GGCGCCCCGG CGGCGGCGGC TCCTCCCGGT CCCGGCGGCC AGCATCACGG CGGCGGCGCC
241 TCCGACTTCA ACAGATACGA GCTGGGCGCG CAGTACTTCG GCGGGGCCGG CCGGTCGCAT
301 TGA
```

(SEQ ID NO:7, Sb01g012870, S. bicolor), or a variant thereof having at least 90%, 95%, or more sequence identity to SEQ ID NO:7.

In some embodiments, the coding sequence of the non-shattering Sh1 gene in S. bicolor, including introns, can be:

```
    1 ATGCCCGAGG ACGGGTACGA GTGGAAGAAG TACGGCCAGA AGTTCATCAA GAACATCCAG
  61 AAAATCAGGT ACTTGCTCCG TTCGATCCAA CATGCATACG TAGCATTTTT TGCATCGAGA
121 TTGATCTCGA GCTCTCACAT AAAGCTAGTG CAAGCTTGTT CACATATACC ATTTTTTCGT
181 GGTCAAATCG TTTCCCGCCA TACGCGTGTA CATCGGATTA ATCAATAGCT CGACGTTGAC
241 CAAGCTTGTT GACTTGTTCA TCTTCGTTCC TGTGCATCAA ATCGTTTTAT TAATTAATTG
301 AGTCGATGTG ACGCCGCCCA TCGATCGAAC ACTGGTATAA TGGAATGTAT GGGTTGCCCG
361 CCGTCCCCGT GCATATATGC ATACGTGCAA TGCTTTGCTG CCAGATCTTA TCTTTCGAAG
421 AAGAATCAAC GGAAGAATAA TATCCTCGCT TTATTATATT ATTGATAACG GTCAACCAAA
481 TAAAAAGCCC TGATGATGAC TTGATGAGCA AACTGCACAA GTGTGTTTTG CATTGCATGC
541 CAACTGATGA TACCGTACGT GGGGTGGTCC ATGATGCATG TGTGTGATCC AAATCCAACA
601 ATGGCGCAGG AGCTACTTCC GGTGTCGGCA CAAGCTGTGC GGCGCCAAGA AGAAGGTGGA
661 GTGGCACCCG CGGGACCCCA GCGGCGACCT CCGCATCGTC TACGAGGGCG CGCACCAGCA
721 CGGCGCCCCG GCGGCGGCGG CTCCTCCCGG TCCCGGCGGC CAGCATCACG GCGGCGGCGC
781 CTCCGACTTC AACAGATACG AGCTGGGCGC GCAGTACTTC GGCGGGGCCG GCCGGTCGCA
841 TTGA
```

(SEQ ID NO:8, Sb01g012870, S. bicolor), or a variant thereof having at least 95% sequence identity to SEQ ID NO:8.

In some embodiments, the coding sequence of the non-shattering Sh1 gene in S. bicolor, including introns and 5′ untranslated region, has the nucleic acid sequence:

```
      1 TTGGTCAACT CAGATGTGCT GAGGTCTGTT TGGTTCTCTT CTCACCTAGG CTACACCGCA
    61 TCTAGAGGGA GAGACAGGCT AGCCACAGCC TGGTCTGGTG CATGCACCTG CACTTGTTTG
  121 GTTTTGCTTT TTGTTTTGAG CCACTCCAGC CATGTCTCGA AAAGATATTG TTTGGTTGGT
  181 CTTTGGCTTG GCACCAGTGC TCTCTCACGT GTACAGGCAC ACGCTCTGTT TTGGCTCCAC
  241 ACAACCATGT GTTGGCTAAA AATGATTTTA GAATCCATTT CCCATGAGCC TGAGATGGTT
  301 GCACGCACTA TGGGCCTAAC CCTGGTAGCA CTTTAGGTAA CCAAACACCT TAAGCCTGCA
  361 TCCCAAGAGC CAGTTTGGAA CTGGACAACC AAATAGGCCT CTAATGAATC TGATGTGTTG
  421 TATTCTGTGC CTGCCTAGCA CTCTTCACCA ACTAAACACC GATAAAAAAA AGTTATGGCA
  481 CGCAATGCCT GAGTGTGGCA TGGCAAGTGA AGGTCGGGAA CCAAACATGC TTTTACTCTT
  541 TCATATCTTA GGCCTGTTTG GTTCGTCGCG GTAAACTTTA ACTTCCATCA CATCGAATAT
  601 TTGAACACAT ACATAGAGTA CTAAATATAG ACTATTTATA AAATTAAAAA CACAACTAGA
  661 GAATAATTTA TGAGACAAGT ATTTTTAGCC TAATTAGTCT ATGATTGGAC ACTAATTGCC
  721 AAATAAAATA AAAATACTAC AATACTTGTT AAACTCTAAT ACCTTCAACC AAACAAGCCC
  781 TTACAGGGAT TCAGATATGT ATATAAAATT ATTTTCGTTA GGCTTTCATA TTAAACTTCT
  841 CATTGTTGTC TCATTACCAT CTTTCCCTGC AAAATGTGAA AACAAGGTGG ACAAATACAT
  901 GAATCCACAT CTGTTCTCAC CCCTAGTATT TAGTAAAAGG AAATAGTGTA CTATCTCAAG
  961 TACAAATAAT GATGTTTCTT CAACACCTCT AACACAAAAT AGTAACTAAT ATTATTTGTG
1021 TAATAATATA TATCTATAAA AGAACATGTT GCCTCTCTCT AGAAAAGTCT ACCTCTTGAT
1081 GTCATTTTCC AAATATCAAA ACTCGATACA CAAAAGAATT GATTTAGAAC CAAAGATTAA
1141 AATGCCTGAC TACATGATGA AACCTGAAAA CATTGTTCTA TTATTAGTGA CTGAAGGGAG
1201 TAATATCCAA CAGTAACTTC TTGTTGCGGA GATTAGTGTT GTACGCAAAA AGAAATATCC
1261 ATATTCCTCC ATATAAAGGA GATGATGAGA TCACAGTGAT TTTCTGGTTC AGTCAAAACC
1321 AGTAGTGTCG AAGTTGGGTA GGACAGCATG TGAACCCAAA AATTTACTGA TTCGTCTTCG
1381 TCTTGACGAT GTTAACGTCG TCGCATCAGA GAAGCTTCCA TTCGATTGAC TAATAAGCCC
1441 TGATAATAAA TATACCACAC CCAAAGAGCT TCGTCACTAC TTTCAATCTC TCTCCCTCTC
1501 ATCTACATGT TTCATTCATT AAACTTTGCG ATAACATGGG AGCAGCAGTA GAGCACAGGA
1561 CGTTGTAGAC GTACGGTCAC TGGCGGCGTC CATGGATTCA AGCTCACAGC CCGGCGCAAT
1621 GTATGCATCT CTCTCTCTCT CTCTCTCTCT CTCTCTCTCT CTCTCTCTCT CTCTCTCTCT
1681 CTCTCTCTAC GCTGTGTTTG ATGCGTTTGC CTTAAACCAG CTTTGGTTCT CATGCATGCA
1741 TGTATGGTTC ATCATGTTTT TGTCAAATTT TCATGTAGCA ACATATATTG TCCTCCGTCC
1801 ACAACAGATA AGCTGATCCT GCTAGTCATA GCTGCTATAT ACAGATCAGC TTATTAAGTT
1861 TGCAGGTTGT TGTTATGCGT GTTCTAATGT TCCTTGGCAC AAAAACTAAC TGTGTAGTGA
1921 TGCACGCAGA GGCAGCGGAG GAGGAGGAGA GAGAAACCAA AGGGAGGAGG ACGAGGCGGC
1981 GGCGGCGGCG GCGGCAGAGG CCGGCTACGG CAGGCAGCTG GTGATGCCCG AGGACGGGTA
2041 CGAGTGGAAG AAGTACGGCC AGAAGTTCAT CAAGAACATC CAGAAAATCA GGTACTTGCT
2101 CCGTTCGATC CAACATGCAT ACGTAGCATT TTTTGCATCG AGATTGATCT CGAGCTCTCA
2161 CATAAAGCTA GTGCAAACTT GATCACATAT ACCATTTTTT CGTGGTCAAA TCGTTTCCCG
2221 CCATACGCGT GTACATCGGA TTAATCAATA GCTCGACGTT GACCAAGCTT GTTGACTTGT
2281 TCATCTTCGT TCCTGTGCAT CAAATCGTTT TATTAATTAA TTGAGTCGAT GTGACGCCGC
2341 CCATCGATCG AACACTGGTA TAATGGAATG TATGGGTTGC CCGCCGTCCC CGTGCATATA
2401 TGCATACGTG CAATGCTTTG CTGCCAGATC TTATCTTTCG AAGAAGAATC AACGGAAGAA
2461 TAATATCCTC GCTTTATTAT ATTATTGATA ACGGTCAACC AAATAAAAAG CCCTGATGAT
2521 GACTTGATGA GCAAACTGCA CAAGTGTGTT TTGCATTGCA TGCCAACTGA TGATACCGTA
2581 CGTGGGGTGG TCCATGATGC ATGTGTGTGA TCCAAATCCA ACAATGGCGC AGGAGCTACT
2641 TCCGGTGTCG GCACAAGCTG TGCGGCGCCA AGAAGAAGGT GGAGTGGCAC CCGCGGGACC
2701 CCAGCGGCGA CCTCCGCATC GTCTACGAGG GCGCGCACCA GCACGGCGCC CCGGCGGCGG
2761 CGGCTCCTCC CGGTCCCGGC GGCCAGCATC ACGGCGGCGG CGCCTCCGAC TTCAACAGAT
2821 ACGAGCTGGG CGCGCAGTAC TTCGGCGGGG CCGGCCGGTC GCATTGA
```

(SEQ ID NO:9, Sb01g012870, S. bicolor), or a variant thereof having at least 90%, 95%, or more sequence identity to SEQ ID NO:9.

In some embodiments, the coding sequence (without introns) of candidate gene Sb01g012880 as it is found in S. bicolor, includes the nucleic acid sequence:

```
    1 ATGGCGGAGC CGGGGCTCGA GGGCAGCCAG CCGGTGGATC TGTCCAAGCA CCCCTCCGGC
  61 ATCGTCCCCA CGCTCCAGAA TATTGTATCA ACAGTTAATT TGGATTGTAA ACTTGACCTC
121 AAAGCAATAG CTTTGCAAGC ACGAAATGCG GAGTATAACC CCAAGCGTTT TGCTGCAGTC
181 ATCATGAGAA TAAGGGAACC CAAAACCACA GCACTGATAT TTGCATCGGG TAAAATGGTA
241 TGTACTGGAG CAAAGAGCGA ACAGCAATCT AAGCTTGCAG CAAGAAAGTA TGCTCGTATT
301 ATTCAGAAAC TTGGTTTTCC TGCTAAATTT AAGGACTTTA AGATTCAGAA TATTGTTGGC
361 TCTTGTGATG TCAAGTTTCC AATTAGGCTT GAGGGCCTTG CATATTCTCA TGGTGCCTTC
421 TCAAGTTACG AACCAGAACT CTTTCCTGGC CTTATCTATC GGATGAAACA ACCAAAGATT
481 GTTCTTTTAA TTTTTGTTTC AGGCAAGATT GTTTTGACTG GAGCAAAGGT GAGAGAGGAG
541 ACCTACACTG CCTTCGAAAA CATCTATCCT GTACTGACAG AGTTTAGAAA AGTTCAGCAA
601 TGT
```

(SEQ ID NO:10, Sb01g012880, S. bicolor), or a variant thereof having at least 90%, 95%, or more sequence identity to SEQ ID NO:10.

In some embodiments, the region between two SNPs that show high levels of genetic association with the shattering trait, located between nucleotide position 11941320 and 1195600 on S. bicolor chromosome 1 including both Sb01g012870 and Sb01g012880, has the nucleic acid sequence:

```
        1 TCTTGCAGTC GATCTCGTCC TAGCTACTTT GGCATGCAGG CAGGCAGGAG AGATCTACCA
      61 AAAGAGTCCT TCTTCCTCCG GCACCCATAT AATAAACAAA ACAAACTACA CGATCGAGAT
    121 CTCGCCAGGA TTTAATTTGA CACGTGCATG GATCACGGTT TGTTGGATCG TCTCCAACAA
    181 TAAGACGAAT GAACTGATAG TACTATATAC GCCTACTACA CCCACCAACG TGCATGGATC
    241 ACACGGTTCA ATTAGTTTGT CTTCCACACG TGCATGGATC TGTGAGTCAT TCAGAATTGT
    301 AGCCTTAATT TGATCAAGCA GTATGTCCAT CCGTTCAAAT GCTCCACTAA ACATATATTA
    361 ATATTTAAGA AGGTCGGAGT TCACATTCAC ATGGAGACTA CTACTCGCTC TGTTTCTAAA
    421 TGTTTGTCGT TTTCGCTTCT CGAGAAATAA TTTTAACTAA ACATATATTA TAAAATGTTA
    481 ATATTTAAGA TACATAATTA GTATTATTTG ATAGATATTT GAATCTAGTT TTTTTAATAA
    541 ATTTATTTAG AGATAAAAGT GTTACACGTA TTTTCTAATA AATTATTTAG AGATAAAGGT
    601 AGTACCGCAC GATGCAAAAA AAAAAACCCA TTAACTGCAC AGGCATGATG CTGGAAGCGT
    661 ACGCCAAATA TTACCTAGCT AGCGCTGGCT GAAGGGTAAA AGAAAAGAGG CAGCAGCTTC
    721 TTGGAACAAC ACACCGCATC GAGGGAACGG TTGCTGACGT AGGACAAGTG ACGTCAGTCA
    781 CGGCTCCAGC CGCGACCTGG CGCGGCCCCC GCCCCGCTAA CGGCCATCCA GGGGTTTAGG
    841 ACGATCGCAG AGCGTGCTTT CAGGTTTGAA TTTGATCGGC ATAAAGTTTC CCTTTGCTTG
    901 AAATTTGTAT ATTCGTCCTT ATAAAATTGG TGTATTATAA AATTTGTTTA GTTCCCAAAA
    961 TTTTCTAATA TTTACCGTCA CATCAAATTT TACGGTACAT GTATGTAACA CTAAATATAG
  1021 ATAAAATAAA AATTAATTGC ATAGTTTATC TGTAATTTGC AAGACGAATT TTTTAAGCCT
  1081 AATTAGTCCA AAGTCTGTTT GGTCAACTCA GATGTGCTGA GGTCTGTTTG GTTCTCTTCT
  1141 CACCTAGGCT ACACCGCATC TAGAGGGAGA GACAGGCTAG CCACAGCCTG GTCTGGTGCA
  1201 TGCACCTGCA CTTGTTTGGT TTTGCTTTTT GTTTTGAGCC ACTCCAGCCA TGTCTCGAAA
  1261 AGATATTGTT TGGTTGGTCT TTGGCTTGGC ACCAGTGCTC TCTCACGTGT ACAGGCACAC
  1321 GCTCTGTTTT GGCTCCACAC AACCATGTGT TGGCTAAAAA TGATTTTAGA ATCCATTTCC
  1381 CATGAGCCTG AGATGGTTGC ACGCACTATG GGCCTAACCC TGGTAGCACT TTAGGTAACC
  1441 AAACACCTTA AGCCTGCATC CCAAGAGCCA GTTTGGAACT GGACAACCAA ATAGGCCTCT
  1501 AATGAATCTG ATGTGTTGTA TTCTGTGCCT GCCTAGCACT CTTCACCAAC TAAACACCGA
  1561 TAAAAAAAAG TTATGGCACG CAATGCCTGA GTGTGGCATG GCAAGTGAAG GTCGGGAACC
  1621 AAACATGCTT TTACTCTTTC ATATCTTAGG CCTGTTTGGT TCGTCGCGGT AAACTTTAAC
  1681 TTCCATCACA TCGAATATTT GAACACATAC ATAGAGTACT AAATATAGAC TATTTATAAA
  1741 ATTAAAAACA CAACTAGAGA ATAATTTATG AGACAAGTAT TTTTAGCCTA ATTAGTCTAT
  1801 GATTGGACAC TAATTGCCAA ATAAAATAAA AATACTACAA TACTTGTTAA ACTCTAATAC
  1861 CTTCAACCAA ACAAGCCCTT ACAGGGATTC AGATATGTAT ATAAAATTAT TTTCGTTAGG
  1921 CTTTCATATT AAACTTCTCA TTGTTGTCTC ATTACCATCT TTCCCTGCAA AATGTGAAAA
  1981 CAAGGTGGAC AAATACATGA ATCCACATCT GTTCTCACCC CTAGTATTTA GTAAAAGGAA
  2041 ATAGTGTACT ATCTCAAGTA CAAATAATGA TGTTTCTTCA ACACCTCTAA CACAAAATAG
  2101 TAACTAATAT TATTTGTGTA ATAATATATA TCTATAAAAG AACATGTTGC CTCTCTCTAG
  2161 AAAAGTCTAC CTCTTGATGT CATTTTCCAA ATATCAAAAC TCGATACACA AAAGAATTGA
  2221 TTTAGAACCA AAGATTAAAA TGCCTGACTA CATGATGAAA CCTGAAAACA TTGTTCTATT
  2281 ATTAGTGACT GAAGGGAGTA ATATCCAACA GTAACTTCTT GTTGCGGAGA TTAGTGTTGT
  2341 ACGCAAAAAG AAATATCCAT ATTCGTCCTT ATAAAGGAGA TGATGAGATC AGCGTGCTTT
  2401 TCTGGTTCAG TCAAAACCAG TAGTGTCGAA GTTGGGTAGG ACAGCATGTG AACCCAAAAA
  2461 TTTACTGATT CGTCTTCGTC TTGCTGACGT TAACGTCGTC GCATCAGAGA AGCTTCCATT
  2521 CGATTGACTA ATAAGCCCTG ATAATAAATA TACCACACCC AAAGAGCTTC GTCACTACTT
  2581 TCAATCTCTC TCCCTCTCAT CTACATGTTT CATTCATTAA ACTTTGCGAT AACATGGGAG
  2641 CAGCAGTAGA GCACAGGACG TTGCTGACGT ACGGTCACTG GCGGCGTCCA TGGATTCAAG
  2701 CTCACAGCCC GGCGCAATGT ATGCATCTCT CTCTCTCTCT CTCTCTCTCT CTCTCTCTCT
  2761 CTCTCTCTCT CTCTCTCTCT CTCTCTACGC TGTGTTTGAT GCGTTTGCCT TAAACCAGCT
  2821 TTGGTTCTCA TGCATGCATG TATGGTTCAT CATGTTTTTG TCAAATTTTC ATGTAGCAAC
  2881 ATATATTGTC CTCCGTCCAC AACAGATAAG CTGATCCTGC TAGTCATAGC TGCTATATAC
  2941 AGATCAGCTT ATTAAGTTTG CAGGTTGTTG TTATGCGTGT TCTAATGTTC CTTGGCACAA
  3001 AAACTAACTG TGTAGTGATG CACGCAGAGG CAGCGGAGGA GGAGGAGAGA GAAACCAAAG
  3061 GGAGGAGGAC GAGGCGGCGG CGGCGGCGGC GGCAGAGGCC GGCTACGGCA GGCAGCTGGT
  3121 GATGCCCGAG GACGGGTACG AGTGGAAGAA GTACGGCCAG AAGTTCATCA AGAACATCCA
  3181 GAAAATCAGG TACTTGCTCC GTTCGATCCA ACATGCATAC GTAGCATTTT TTGCATCGAG
  3241 ATTGATCTCG AGCTCTCACA TAAAGCTAGT GCAAACTTGA TCACATATAC CATTTTTTCG
  3301 TGGTCAAATC GTTTCCCGCC ATACGCGTGT ACATCGGATT AATCAATAGC TCGACGTTGA
  3361 CCAAGCTTGT TGACTTGTTC ATCTTCGTTC CTGTGCATCA AATCGTTTTA TTAATTAATT
  3421 GAGTCGATGT GACGCCGCCC ATCGATCGAA CACTGGTATA ATGGAATGTA TGGGTTGCCC
  3481 GCCGTCCCCG TGCATATATG CATACGTGCA ATGCTTTGCT GCCAGATCTT ATCTTTCGAA
  3541 GAAGAATCAA CGGAAGAATA ATATCCTCGC TTTATTATAT TATTGATAAC GGTCAACCAA
  3601 ATAAAAAGCC CTGATGATGA CTTGATGAGC AAACTGCACA AGTGTGTTTT GCATTGCATG
  3661 CCAACTGATG ATACCGTACG TGGGGTGGTC CATGATGCAT GTGTGTGATC CAAATCCAAC
  3721 AATGGCGCAG GAGCTACTTC CGGTGTCGGC ACAAGCTGTG CGGCGCCAAG AAGAAGGTGG
  3781 AGTGGCACCC GCGGGACCCC AGCGGCGACC TCCGCATCGT CTACGAGGGC GCGCACCAGC
  3841 ACGGCGCCCC GGCGGCGGCG GCTCCTCCCG GTCCCGGCGG CCAGCATCAC GGCGGCGGCG
  3901 CCTCCGACTT CAACAGATAC GAGCTGGGCG CGCAGTACTT CGGCGGGGCC GGCCGGTCGC
  3961 ATTGACGCGG GGAGCCAGGG TCTTGTTTAC TTTCTAAAAT ATTTTATAAA AATTTTCACA
  4021 TTCTTTATTA CATTAAATTT TGCGGTACAT ACATGATGCA CTAAATATAG ATAAAAAAAA
  4081 TAACTAGTTA CATAGTTTAT CTGTCATTTG TGAGACGAAT CTTTTGAGCC TAATTAGTTT
  4141 ATGATTGAAC AATATTTGTC AAATACAAAC GAAAGTATTG ACAAACCGAC AAGAAAGGCC
  4201 GGCGGCGTTG CGTCACGTAC GCATGCATCA GCTCCTGTGC TGGCCTCTGC TGGCTGCCGC
  4261 TGCATCGATC GATCGCTTTC GCTGCGCACC GGAGGGCAGC GGCAGGTGCT GCCGGTGCCG
  4321 GTTGACGCCT TGCGCCGGCG CAACGTGATG TTGAGTGCGG ATTAATTGTT GCTGCTCCGG
  4381 TTAACTCTCT GGTCTAGTGC TAGTGTACGG CTACTATTAG GACGATGGTG CATAATTGTA
  4441 ATTTTGATAT TGTACATGCA TAAAAAACAA TATTTAGCTG AAAGTGGGAA GTAGCACCGT
  4501 CGCTATTATG TTTTGTTTTC TGCAAAGTGT AAACTTGTCG AAAGTCTCCA GAGTTGGGTT
  4561 CGAGGCCCTG GTCACCCAGT TTACATTGCA TCGCCTCTGA ACTGAATGCG ACACTCGAGA
  4621 CCTAGCTTTA TCAGTGGGAT ACACCTAATT CGTTTAGTGA GCGTTTAACA TTCAATCATT
  4681 TGCAGATAAC CTGGCAGCTG ACACTGCAAC GGCTGGGTAT CCACAACCAA CAAGTTGGCA
  4741 ACACTAATAA TGTTTTCGAT TGAGGTAAAC ACCGAAGAGC GGTAAACAAA GTTCCATGCG
  4801 ATACGAGACA GCTCGTTCGC CTAGCAATCT GGAAAGACAC AGTAATAGGC ATTCTTATAC
  4861 AGTACGTACA ATTCAAATTA TTCATCCTAG CATACAACAA CATCGAAAAA GTTAAAAACC
  4921 ACAAGTGCAG GAACATTTGG ATACAGAAAC ATGTCTACTG CGTGGTCAGT CGACCGGTTC
  4981 CTCCATACGG TGATAATAAC CAACAAGATT ATTCCCGGTG TCCTCTACGA TACAGCATCT
  5041 CAAATACAAC AGATAACTTA CAACCAGTCA CACTCACACA ATCCCGTCAG TAGTCAGTAC
  5101 ATTGCCCCAG TTACCTACAG TGCCAGTCTT TTCATCATCG CACAGCACTG AAAGATACTC
  5161 AGAAAAGACT TTAATAGACT CGTGTCTCAA AGACAAAGTA GGGCAAAATT TATCTACTCT
  5221 TGTTAGCACT CAAGTTAACC ACATGGGACA CAAACTACTC AAACTGAAGT AATTTGACAA
  5281 GTCCACCAGC TACCACAACA AACCCACCCA ACCATGACAC ACCGAGGCTC ACAGAAATTA
  5341 CAGGATGCTA TAAGTTCCGC CAGACTTTTT ATGTACAGTT AGAATTTATG GTCACACAAA
  5401 AAACCTCAAG GATGCTTGTA ATTAGAAGAA CGTGACCTTC ACTTGGGTCA TCTGCAAAGA
  5461 GGGCACCAGA AGGAAAAGAT TAGTTTTAAA TAATTAATTC TAGTACTGCA CACACCGACA
  5521 CGAGTTATAA ACAATATAAA CGGTCCATTT GGAATACAGA AATTTCACAG AAATCATGTA
  5581 CAATTCCAAG GGAATCGGTC CATTTTCACA GGAAAACACA GGAAACAGGG GGATCCCACA
  5641 TTCCAAAAGG GGCTTAAAGA GAGAAGGAAT TATCCCACAT TACAGGAATT AACATGCCAT
  5701 GACATCTGAT TTGAATACCT AGAATACCAT AATAAAAGTT TGTTTCGAAA ACACAGTAGA
  5761 AAACATGATT CCAACATTTT ACTATCAAGT CTAACAACAA ATAACATATA GGTGCCCAGT
  5821 CCCACACATG TTCCAAAAAT GAGTACAAGA CATAGTGAAC ATAGTCAACA GAACAAGAGA
  5881 ATCTCAATTG CAGGAAGAGT CATGCATGCG CTATGATTGA AGCATGATAA AAAGAACTAC
  5941 ATACCATTGC TGAACTTTTC TAAACTCTGT CAGTACAGGA TAGATGTTTT CGAAGGCAGT
  6001 GTAGGTCTCC TCTCTCACCT ACAAACAATC GACTATGAAA TTAAGGAGAA AGATAAGCTA
  6061 ATCGCAGTAT AATTAAGCAT GAGCACGAAA TGACAACTAA CCTTTGCTCC AGTCAAAACA
  6121 ATCTTGCCTG AAACAAAAAT TAAAAGAACA ATCTTTGGTT GTTTCATCCG ATAGATAAGG
  6181 CCAGGAAAGA GTTCTGGTTC GTACTGTAAA ACAAATTAAA AATGTCATTA TCCAAAGAAT
  6241 GCAGACAAAA AAGGGTAAAA GAATTACTGT GATGTTAAAA TAAGCCATCA TTGGACATAC
  6301 ACTTGAGAAG GCACCATGAG AATATGCAAG GCCCTCAAGC CTAATTGGAA ACTTGACATC
  6361 ACAAGAGCCA ACAATATTCT GAATCTTAAA GTCCTGGCAT AGAACAGTAA CTTAGCAACT
  6421 GATGTACAAA TTGTTCAAAG TACAGGTCAA TGTACACAAG TATGAAAATA GTTACCTTAA
  6481 ATTTAGCAGG AAAACCAAGT TTCTGAATAA TACGAGCATA CTGGAAATAC AGACAGGGGT
  6541 TAGAATTCCA AAGCTCTCAG TAAACTAGAT CCAACTTAAA TAAAATGGTA GCAAGCCATA
  6601 TGGCACCTTT CTTGCTGCAA GCTTAGATTG CTGTTCGCTC TTTGCTCCAG TACATACCTG
  6661 GTGATAGAAA ATTATCGGTT GCTTGCTTCA GCACTAGAAC ACTTATGATG GATTGATACA
  6721 AGATTGTAGT TCTATATGAA AGAAATGCAG TTCTAGTAAA CTTTCTTCAT TTGGAAGAAA
  6781 AGTATTGACA CATCAATGCA TTTAATTAAT ATTCAATATG ACAACCAAGA AAGTCTACAA
  6841 TACTGACTAT TGATCCAAAT AAATCCCAAG TAAAAACCCA CCGAGATATA TCATCTGGTA
  6901 AGGGAAAATA GATTTGCCTA GGGTAGGCTA GAGAGGGTAA GAACTTTATT CTCCAATATT
  6961 TGATGATTGA GAGAGGTAGA TTAGGACACA GAAAAAACAA ACAGATTAGC CTTTCTATCT
  7021 TTTGACAGGA CAGCACCAAG GCAACAAAAC ATGTCAAAAA AAAGATCAAA TCTGTTTACA
  7081 TCAAAAACAT GCAAAATCCT TGAAAATTGA CAGTATAAGA CAAAAGATGT TGATGACATA
  7141 CCATTTTACC CGATGCAAAT ATCAGTGCTG TGGTTTTGGG TTCCCTTATT CTCATGATGA
  7201 CTGCAGCAAA ACGCTGTTTA CAGATAAAAG AGTCAAATAC GAAATATAAT GACAGAAAAC
  7261 TTAGCAAAAT TCAGGTTGCT ACATTGTATC ATCATAACTG AGAAAGATTG CATTCAATAG
  7321 AATGCCTAAA AGAGCAAACA AGTCATATAT AAGCTAAAAA TTTAGAACTT GATTGTCAAA
  7381 GAATATTGTG GTTATTCACA GGACAAGCAG GATATGAGCA TCCATCTGGT TTGAAACTAA
  7441 CCGTGCACAT CTCATATCCC AGGCCATCCA TTAGTTATTA GCACAAAGCT ATTTGAACTC
  7501 ATGGACAAGA TTGTACATCA TTACAAAGGA TCAACATACT TTATATGTCC ATAAATCTTC
  7561 CACTAGATAA AAACAACAAG TAAATACCGT GCAAAGCCAT TGCTTTGAGG TAATCACTAT
  7621 ACCTTGGGGT TATACTCCGC ATTTCGTGCT TGCAAAGCTA TTGCTTTGAG GTCAAGTTTA
  7681 CAATCCAAAT TAACTGTTGA TACAATATTC CTGTCATGAA AAAATGACAC ACGTCAAGCA
  7741 GACCATGATC AAAGAACTGC AGTAAACATG TGAATTTTGT TTTGTAAAAC CAACATAGGG
  7801 TTCTTATTGT AAGTTTTTAG CATTGAAGAG ACACTACAAG ATAATTTTCA TTGTTCTTTT
  7861 TATATTTGAT AGTGTGTGCT ATTAATTTCT TCATGCCAAT TTCCAACATG TGCAAATCAT
  7921 AATAAATTTA AGACTAACAT TCAAGATAAC CTACACTATA ATGGTTGGAT CGTAAAATCT
  7981 TTGTATCAAT CAAAGTCATT TCAGGACTCA ATATGGCACT AATATGCCCA TAGCACTTAA
  8041 TAATGAAATT GCCTGCAGAA AAATCTTACA CCTAAATCAT AATAAAAATC TTCCACAAAA
  8101 GCTAGTTAGG TTACTTCTGG TTTGGGGACG GAGTGGGATG GAATGGTCAT GTCCCTATTT
  8161 TTTGGACGGG ATTGACCCGG ATCTTGTTTG GTTGGACAGA AAGGTTCATT CCAATTTTTG
  8221 TTTGGTTCGA AGGATATGGT GGGATGGAAC CCGCTGGAGT TTTAACTCCA TTAGACACAA
  8281 TAATCCATGG CCGCACCATC CATTGTCTCT ACACCTGTTC TTGTTGTCTT CTTCAGGTGA
  8341 GCAAAGCATG ATTCCCAAGA TTTTGTACCA CAGTCGCTCA ACATCTCACA GCTCCGGTGC
  8401 CCAACAGCTG GGCACTACCA CCGCCCAAGA GCTTGGCCAA CCCATTCGCC CAAGATCTCA
  8461 TGCAGAGATC TTGGCATTGC CACCACCAGA GATGCTCAAC CTGCCCCACC AGAGTTCTCA
  8521 TGTGGCCAGA GGAGGTAATT GGACCCACTC CTCTTATCGT CGGCGCTAGC CCAGTGGGCT
  8581 GCATATGCTC CAAACATCTC CTCTCCTCCG CTTGCCTTGA GCTTGGAGCT TCCACGTGCC
  8641 TGCGCCCCTC CTTTTGACCA CGCTTGCACC AGGCAATGCA AAGATGGCGT GCAACGCCGT
  8701 CCGCAAGGAA TGGCTTCATC CACCCGATTC AAGGGGACCG AGCTGTCCAC ATATTTCAGG
  8761 AATATGCCAC TGCAAAAAAT GACCCCATCC CTAGCTCCTC CCAACCAAAC ACTGCTGAAA
  8821 AAGGATTGCC CCATCCCGTC TGGGACGTCC CTCAATCCAA GCCAATGCAT TTAACCCTCC
  8881 CCACGATATA AGATATGGAA ACCTCAGTGC GTGAGGCTGA CTGTTTATCA TATTACACAA
  8941 TTTATGCACC AACGAGTCAA AACATAGAAT GGAAATATGG TAAGAAGAGA TTATGCTTGC
  9001 TGCAACTATT ACGCCAAGAT GACAAACTTC AATAAGGAAA TAGATCTCCT CTCCAGTTTG
  9061 GCCCTCTCTC GTTCTCCCAA GTTTCATACC TGAAATCAAC CCTCGGAGAG AGGATGACAA
  9121 CTAAATAATT CCCACCAAAG CCCCAACTAT TTAAGACAAT ATTAGCTCGT TTCGATGCAC
  9181 CCAGCACCGG GAAGCTGAAC AAAAACACGG CATAAACCAA CCACACCACC ACCCACAAGA
  9241 CAGGGAGGCA CCCCGCTGGC CAGAACCAAG CCTTGGCAGC TCCACAGCAC ACCCAAGCAC
  9301 CCATCCGCCG GGCGGCGGGA CCCTAGCACG TACGGTACGG GATCTCTCCG GAACCCCGAA
  9361 TCCCCGACGA CCCAGATCCG GGACTTACTG GAGCGTGGGG ACGATGCCGG AGGGGTGCTT
  9421 GGACAGATCC ACCGGCTGGC TGCCCTCGAG CCCCGGCTCC GCCATCCGAA CCACGCACGC
  9481 GACCTCGGCG GGGCTCCGCG CCGCGAATCC GGGGCCGAAA TGGGCGGGAA AGGAGCGCGC
  9541 GCGTCACCGG TTCGAGGGGG AATTCGAAAT CCGGGTCTTT TATAGAGATC GGGAGAGGAG
  9601 TTGGGGAGGA GGGAAAGCAA GGGGAAGGAG AGCTAGGGTT ATCTGTCTCG CGAGGGGGAG
  9661 TCGGGGACAG CGCAGGCGGC GTGAGAATGC GGGGGGAAGA GGGGGAGGTC GTCTGGTGGT
  9721 GGGAGGTAGA TGCGTGCGGG AGTTGGGGTT GTATCGGTGG ACGGGGAGCA GGCGGTGGAT
  9781 GGCGACTGCT TGGCTTTTGT AGGGGAACAG GGTGCACCGG CTGTGGCCGG TTACCCCAGG
  9841 GCGCGGTTTG CCCACGCGCT GGTTCGAGTT ATGCAAACTG ACCTGTGGGT CATAGCATGC
  9901 GGTGGGACCC GGTGTCGGTG TGTGTGGGTA TGATGCGCGT TCGACGGCCA TTAATCAAGA
  9961 ATTTCTCCTA CTCGCAGATC GCACTAGCAG GTTTACGAAC GCGCCGAGAA GATCGCACTA
10021 TTATGAATTA TTTTCTTTGA AAGAAAATTG TTATGAATTA TGAAAATCAT GAACTATACT
10081 AATCGGACTA TTTGAATTAT TGTGATGGAT CATTTTCCGT TCGAGTGGGA AATCATGGTC
10141 ACCAAAAAGC TGGTAAGAGA GAGATTATAA GATGATTATT ATAGTCGAGT GTTTTAGTTA
10201 TGTTTAGTTT ATAATTAAAT TATTTTAGCT AATTATTATA ATCACAGTGG ATCCAAACAG
10261 GCCTGACTAG TGACTACTTG AGCATTCGCG TTACGTCGCT GTTGCAGTGC ACATTCATTA
10321 ATGTTAAGGC CTTGTTTAGT TCCCAGAATA TTTTGTAAAA ATTTTCAGAT TCTTCCATCA
10381 CATCGAATCT TGCGGCATAT GTATGGAGCA CTAAATATAG ATGAAAGAAA TAACTAATTA
10441 CATAATTTAT CTGTAATTTG TGAGATGAAT CTTTTGAGTC TAATTAGTCT ATGATTAGAT
10501 AATATTTGTT AAATACAAAC GAAAGTGCTA TTGTTCCTAT TTTGCAAAAA AATTTGAAAC
10561 TAAACAAGGC CTAACTAAAA CATCTTGCGT TAGAGCTTCC TTGATGCACC ACGGTGGCGT
10621 GCTGTCGTAG TGACCACCTC AGCTCTAGAC TTCCATGTCA TAGGCTCTTG CAGAGGAGAT
10681 CATGGCCTCA TCTAAAAAAA ATCAAAGGCA ACAGCTAGGC AGCGTGCTAT GGTGGAAGTA
10741 GTGGCTCTAA GCTATTGGGA CCACGTCTGG TTCGTGCATT TGGCTCCAAA TTGTCTTTAG
10801 CAGCGACTGA CGGTGGAACG CCTATAGAGA CAAGCCACAT GCAGCTTGCA TTGAGTACAA
10861 TGGTGGTTTT AACTTTTAAC CCATCGAACG TACGTGGATG GTCACCTTTT TTTCCTGGGG
10921 CTAACGCTAC TAGGTGCCCG TGTTGCGACT ACCCTTAGGC TGTCTCCAAA GGCATGTGAA
10981 ATTTTTTTGG ATTTCGCTAC TGTAGCACTT TCGTTTGTTT GTGATAAATA TTGTTCAATA
11041 ATAGACTAAC TAGGGTTAAA AAATTTGTCT CACGATTTAC AGTCAAACTG TGTGATTAGT
11101 TTTTGTTTTC GTCTATATGC TTCATGCATT TGCCGCAAAA TTCGATGTGA CAGGGAATCT
11161 TGAAATTTTT TTGGATTTCA GAATTAACTA AACAAGGCCC AAGACCCATT TGGGAACCCA
11221 AATCCAAAAT AGGTTTTCAA CACAATACCT ATAGCCTCCA ACAGAGTACT CATACAGAAG
11281 ATCCATTTTG AGTATCAGGA GAGGCATAAC CCAAATTTGG GTATCCTCTC TCTTCGAGAC
11341 CCATTTGTAG AGAGTGTTGT CTTTTAGGTC TTGTTGTTGG AAAAGACTAA AAATAGGTAT
11401 GGATCCTTTT AGCTGTAGCG CTAACCAAAT GACAAATGAG TTTTGTATTT TGGGTGACGA
11461 TTGTTGAAGA CAGTCTTGTA CTAGCCACAA CGGCGAGCAT CGATGTGTCA GTAAGCATGT
11521 CAGTAAGCAT CGGTTTATAA GAGAGCTGTA ATGTCTAAAC ATCATGTGGG ACCAACCAAA
11581 TGAATAAACA AACAAGGAGA CATTGCAATG CCTGAACATA TCAGTGAGCA TCGGTTGAAA
11641 CTCGCCCTCT CTCAGTATGT GCAACTATAG TTTTTTTATG TTGCACTGTG GAAAGTAGAA
11701 GCCTCGATGT CGCACAAAAA AAAATCAGCA TCGCACCCCG CGATGTGATG CCTCAAGGCT
11761 AGAAGCCAAA ATATGCGCAA TGGTAAAACT ATACGTTATG TGTAGTCTTA TATATAAAAT
11821 GTTAGAAAAA AATATTTCAT TTTAGAATGG AGAGAGTAGG CAATAAGACC AGTACAAAAC
11881 GGACATAAAT CTAAAACAAA TATTGTTTGA GAGAAAATAT CTAAAATCAA TCCAAGTATA
11941 AGCAAGCATC ATATGTGACA TAATAAGAGA TTAATAATCC TAAAATGAGT GTACATGTCT
12001 TGCATCAATT TATGAAACTC GAATTATCTG TCTCCCAGAG CACGAGCCAA TGCCACTCAT
12061 AACCTATTAC ATATAGGTCA ATCTTTTACA GAGCTTGTGA TCATCTTTAT ATCTGATCAT
12121 CATTTAACGA TCTGCGGGAC TAGTAGGCTA TCAGAAGCAA TAACCTTCGG TTGTTTCAGA
12181 TGGACACGAA TGTGCATCAC CAGTTTACAG CTCTGTATAC TTCACCTAAT AACTGAACAT
12241 TCTGAGAGAA TGAACTATTT GTGGCTCCTT GATGAGGCCC AGCATGTTTA CCTTTTAGGT
12301 TCCCTTAGGT TAAACACTAA ATCTTCATGA TGGAAGGTGT TTGCCTGAAC TCCAAGACAG
12361 CAAGGTTTTC TCTATACTTC TTTACTTCGG CCACCATTCT GTCGTACGAT TCAGGGTATT
12421 TGCAAAAAAT CACGATTTTG ATTCAGCTCC CTGGCTCGTG CCTGCAATGT CAACATGATC
12481 CTTTACAAAT GTTCGAAGGC ATCCATTAAT TACCCGAGGG GCACCACCAT CACAAAATCG
12541 CTTTGCCAGA TCTACTGCCT GAAAGACAAG GGTCGAGAGA CATTTATATT CTACTAGTAC
12601 TCAAAAGTGG AAAGAGTAAT AGCTATAAGA AAACATGCAG GTGCTTGATG CATAAAGTCA
12661 AAATATGAAG AAAAACAAGT AATAGGGAGA AATAAGCACC TCATTGATGA CAACTTTGTG
12721 AGGTGTTCCT TTTGATGTCA TCTCTGCCAT AGCAATATGT AGAATGCAGA GCTCAAGTAT
12781 CCTTGCCACA GGCTCATCCT GCCATGAAAT TTTCCATGTA TCAACAGCAG GTTATGCCAT
12841 AAAACAAGAC AGCAAAATAA TAAATACTAA AATATAACAC CAAGTTAAAG ATCAGGAAGA
12901 TTATAAACTG ATGAAAGGAA AGTAATATAT TGTGTTTGAA CCAAACACAA TATAAACAGC
12961 TTGATGCATA TCGAAGGGAT TTGATGAATC AACATAGAAT AGTAGGAAAA GGTATCTAAC
13021 CTTCCAAGCC TGGGGAATTA TTTTGTCAAT GATATCTACA TGCTTATCCC ATCCACTAGC
13081 AACAGCCACT AAAAGTTCCC TGGACAACCT GTACTGGAAA AATATCTAAT TAGGAATGTA
13141 AGAGCAGCAG GACTAAATAT TAAACAGGAA ATTAAATTTT ATCATATATC AGAACAGTGT
13201 ATCGATACCT AATGCCTTTA GTGGAATTGG GCAAGAAGGA AAGTATACCG TAAGACAAAG
13261 TTGTTGTACA CCAGTTTTGG AGGAGCTGAA AGTACATCTT CTTCTGAATA TGAAAGAAAA
13321 ACATGTCAAA TTCTTTGCAG AAGAATAACC AAACATTAAT GGAACATATT TACACAAAAA
13381 CAAATCTATA GTTACTCAGC TGATTTCACA ACAGACTAAG GAAGAAAATG TATACGGTTA
13441 ATATGACTAT ATGAGCCGTT TAGCACGCAT CGTAAGGATA TGTTTATTGT GCTGAACGAG
13501 ATAGATGCCA CTGGGCTGCT ACAAAAGATG CATGCTAACG AACGTGAACA GTTTTCAGCA
13561 TGTCGATTAA AAGTGTAATC AACACATAGC TTGATAAAAT ATATCAAAAT TTACTGGCGC
13621 TTAGAGTGAT GGATTATGGT ATAGCTCTCT TAAAACTCAG TCTGCAAAAC CACCAAAAGA
13681 AAAAAAAAAC AGATACACAA CCCCTGTAGA TCTTAATGAC CTAGCCTGAC TAGGTAGCAC
13741 CTAGGCATTA GCCACTATAC CGAATCAAGA GTTAGGTGCC ACACAGCTGC TTACCTAGCA
13801 CATTGGGTTT TTTAAGCCAA AGCACTGCAT TAACTGTTGT AGTTTAACGG TCTGAAATTC
13861 ACAGCACCAA CTGTGAATTG CTCTAGCATG CCCTCCAGTT TTTATATACA TGAAAATAGG
13921 CACACGCCCA CAATAAAAAA AAAAAGAAAC TTGGCCTAAG TTCAATAACG TATTTATGGA
13981 ACAACCAATG ATCCATTGCT CTCTTTACTT TAGGAAACCA GAATCATAGA TATATGACGA
14041 AAGTTTCAAA ACTTAGACTG AAACCCACCA TAAAATTTGT TTAAACAGGA ACCAACTAGA
14101 TTTTCTGGTG GTTGTATGTT TCAGATTGAC CGAAGGATAA CCATTAAAAG ACTGCTATAA
14161 TGGAATTGGT ACCTAACTGA ACTTGTGCTC TTTGGAATCT TCTGGATATA GAAATATTGA
14221 ATCTCAAAAT TGTGAAAAAA AAAGATGGGC ATATGTCCAA ATTTACCAAC AACAATCTAC
14281 GACTCCAACT GTAACAGCGT TAACATATAG GAAGTAGCTA TGTTACCCCG ATACTTCTCT
14341 GAATCGCCGT ACCGATATCG CGATACGTAT CCGATACGGC GCCGATACGG TATCGGAGAA
14401 GTATCGAGGA AATAGAGAAA TAAAAATAAA TAAAATAAAT CCGATACTAG ACCGATACCT
14461 TCCCGATACT TCCCAGCCCA TAACCTCTCA AATTGAAGTC CATCAAGTTA GCAGCTCATT
14521 TTTGTGGCCC ATTTACACAA CACTAAAACC CTACTAGCCA CCACACGTAC ACAATAGATG
14581 TAGTAGCGGA CTTAGCCTAA AACTTATAGT ATCCTAATAT TTATTTTTCT GCTGTAAGGA
14641 TATTAAAAAC AATATTTAGT TTTCTGCTGG TGTGAAACCA AATA
```

(SEQ ID NO:11, Sb01g012870 and Sb01g012880, S. bicolor), or a variant thereof having at least 90%, 95%, or more sequence identity to SEQ ID NO:11.

Accordingly, in some embodiments, a nucleic acid sequence containing the Sh1 gene as it is found in S. bicolor includes the nucleic acid sequence of SEQ ID NO:7, 8, 9, 10, 11, or a fragment or variant thereof.

A polynucleotide is disclosed having a nucleic acid sequence SEQ ID NO:7, 8, 9, 10, 11, or a fragment or variant thereof. Also disclosed is a fragment or variant of the Sh1 gene as it is found in S. bicolor having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 7, 8, 9, 10, or 11. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more nucleotides shorter than SEQ ID NO:7, 8, 9, 10, 11.

Also disclosed is a polynucleotide that hybridizes under stringent conditions to a polynucleotide consisting of the nucleic acid sequence SEQ ID NO: 7, 8, 9, 10, 11, or a fragment or variant thereof.

B. Polypeptides

1. Shattering Sh1 Polypeptides

An amino acid sequence encoding a shattering Sh1 gene product is also disclosed. Thus disclosed is a polypeptide encoded by the nucleic acid sequence of SEQ ID NO: 1, 2, 3, 4, 5, 6 or a fragment or variant thereof. Also disclosed is a polypeptide encoded by a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1, 2, 3, 4, 5, 6 or a fragment or variant thereof. Also disclosed is a polypeptide encoded by a polynucleotide that hybridizes under stringent conditions to a polynucleotide consisting of the nucleic acid sequence SEQ ID NO: 1, 2, 3, 4, 5, 6 or a fragment or variant thereof.

A polypeptide that is a fragment or variant of a shattering Sh1 gene product is also disclosed. Thus, a polypeptide encoded by a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1, 2, 3, 4, 5, or 6 or is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more amino acids shorter than the polypeptide encoded by the nucleic acid sequence SEQ ID NO: 1, 2, 3, 4, 5, or 6.

In some embodiments, the shattering Sh1 gene product as it is found in S. propinquum includes the amino acid sequence encoded by SEQ ID NO:1

MDSSSQPGAI DTCRGSGGGG DRNQREEDAA AAAAAEAGYG
RQLVIPEDGY EWKKYGQKFI KNIQKIRSYF RCRHKLCGAK
KKVEWHPRDP SGDLRIVYEG AHQHGAPAAA APPGPGGQHQ
GGGASDFNRY ELGAQYFGGA GRSH

(SEQ ID NO:12) or a variant thereof having one or more conservative amino acid substitutions and at least 90%, 95%, or more sequence identity compared to SEQ ID NO:12.

In another embodiment, the shattering Sh1 gene product as it is found in S. propinquum includes the amino acid sequence of the polypeptide encoded by SEQ ID NO:5:

MAEPGLEGSQ PVDLSKHPSG IVPTLQNIVS TVNLDCKLDL
KAIALQARNA EYNPKRFAAV IMRIREPKTT ALIFASGKMV
CTGAKSEQQS KLAARKYARI IQKLGFPAKF KDFKIQNIVG
SCDVKFPIRL EGLAYSHGAF SSYEPELFPG LIYRMKQPKI
VLLIFVSGKI VLTGAKVREE TYTAFENIYP VLTEFRKVQQ

(SEQ ID NO:13) or a variant thereof having one or more conservative amino acid substitutions and at least 90%, 95%, or sequence identity compared to SEQ ID NO:13.

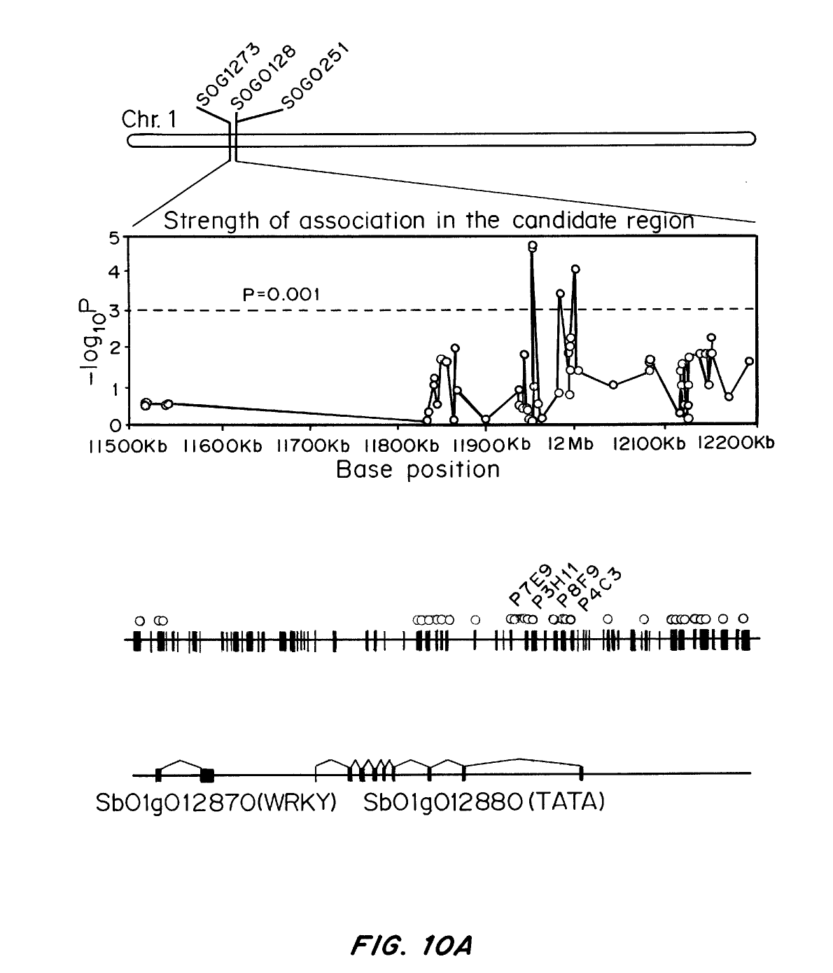

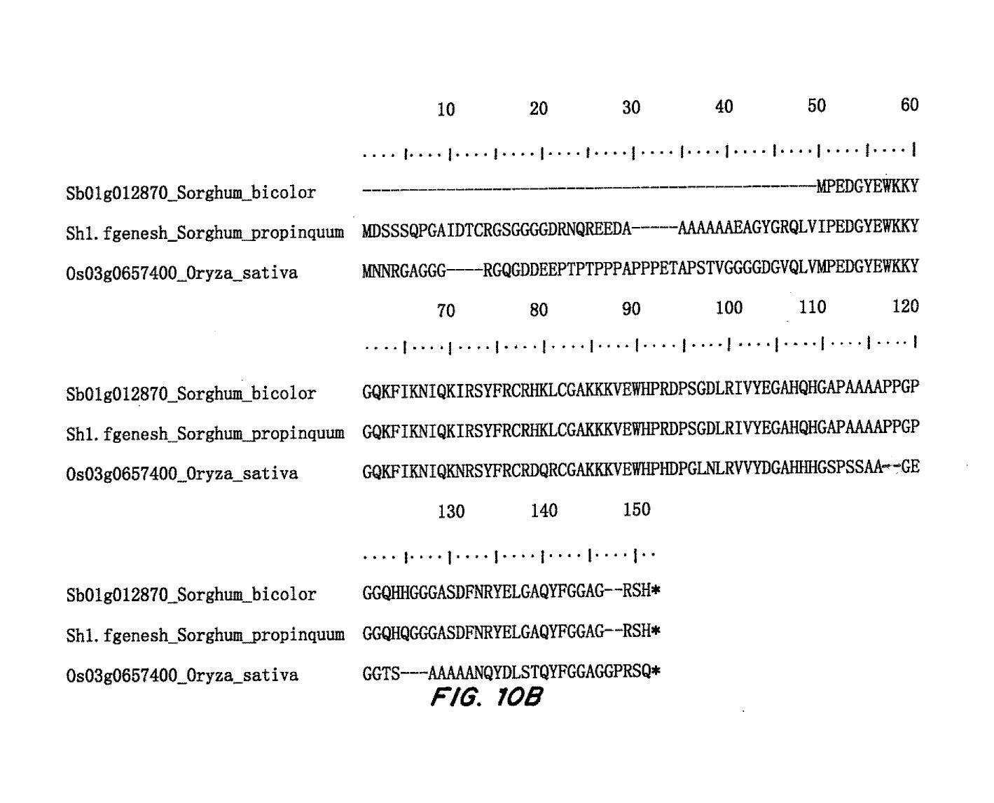

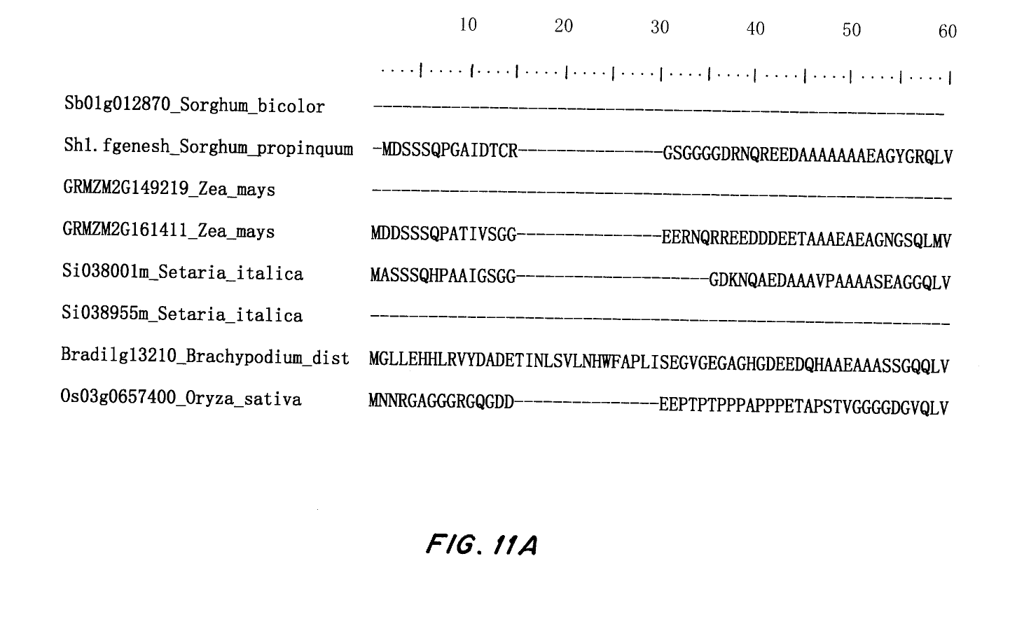

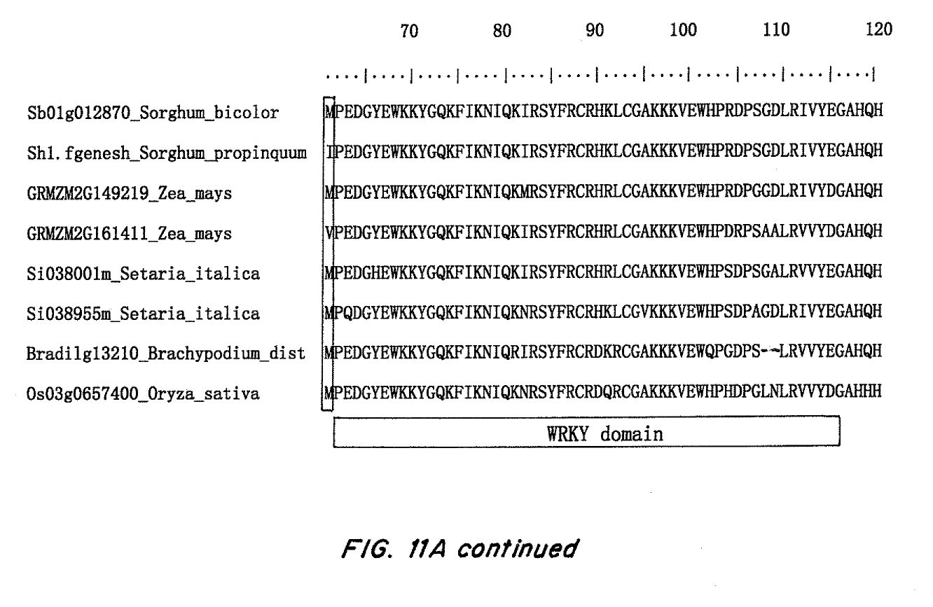

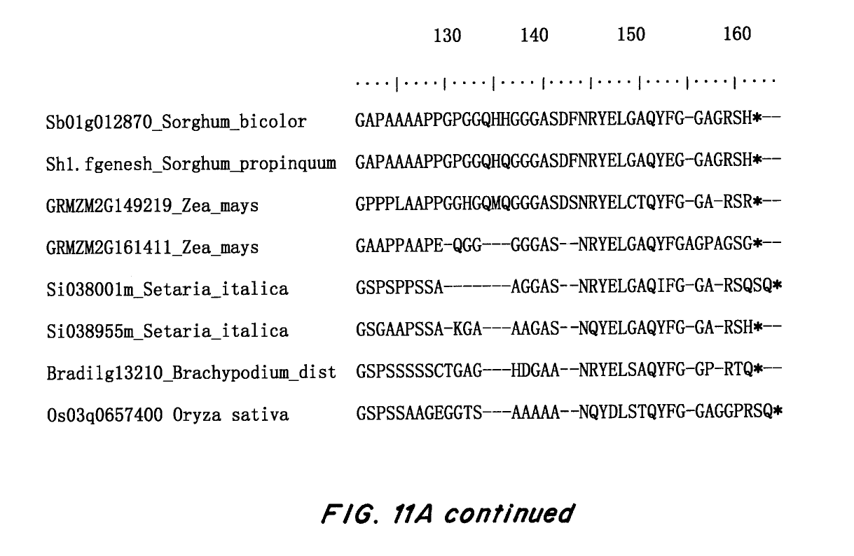

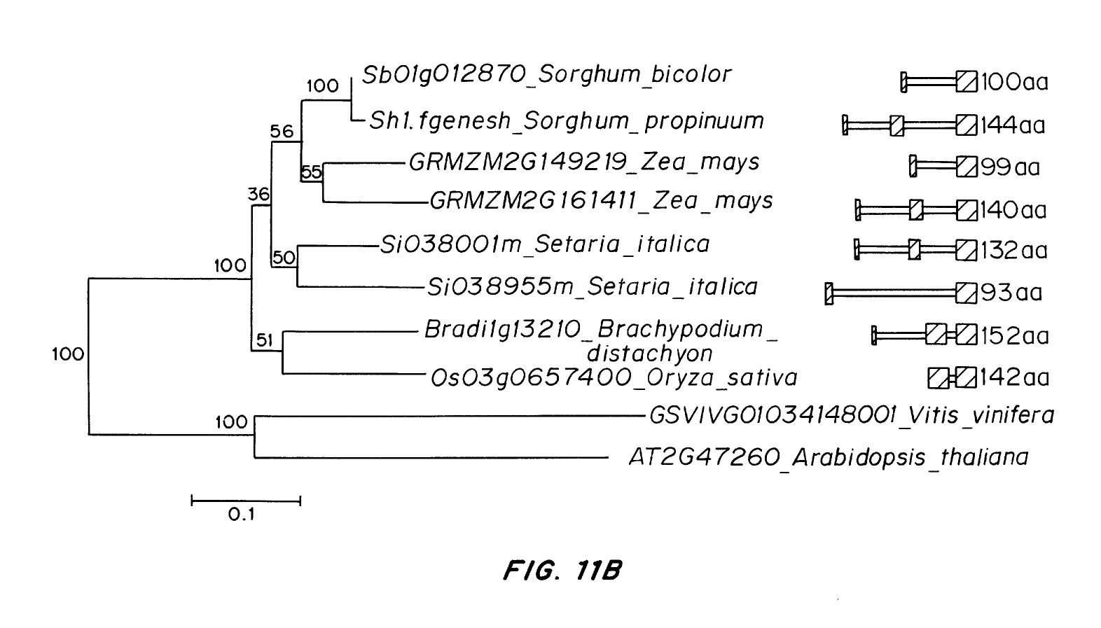

SEQ ID NO:1 is the nucleic acid sequence in S. propinquum homologous to the predicted gene sequence Sb01g012870 (SEQ ID NO:7) in S. bicolor. SEQ ID NO:1 encodes two non-synonymous mutations relative to SEQ ID NO:7. An G→T at nucleic acid position 3; and C→G at position 228 of SEQ ID NO:390%, 95%, or more relative to SEQ ID NO:1. The transversions result in methionine (M)→isoleucine (I) and histidine (H)→glutamine (Q) missense mutations at positions 1 and 76 respectively of SEQ ID NO:16 relative to SEQ ID NO:12. The amino acid sequences are aligned in FIGS. 10B and 11A.

The methionine (M)→isoleucine (I) mutation results in a change in the translational start site of the S. bicolor allele, which makes the S. bicolor protein 44 residues shorter than the predicted S. propinquum protein (FIGS. 10B and 11A). The 44 amino acid fragment is:

### MDSSSQPGAI DTCRGSGGGG DRNQREEDAA AAAAAEAGYG RQLV

(SEQ ID NO:14). The 100 amino acid fragment in S. propinquum homologous to the predicted gene sequence Sb01g012870 (SEQ ID NO:7) in S. bicolor is

IPEDGYEWKK YGQKFIKNIQ KIRSYFRCRH KLCGAKKKVE
WHPRDPSGDL RIVYEGAHQH GAPAAAAPPG PGGQHQGGGA
SDFNRYELGA QYFGGAGRSH

(SEQ ID NO:15). Accordingly, in some embodiments, an amino acid sequence encoded by the Sh1 gene as it is found in S. propinquum includes the amino acid sequence of SEQ ID NO:14, or 15, or a fragment or variant thereof.

A polypeptide is therefore disclosed having the amino acid sequence SEQ ID NO: 12, 13, 14, 15, or a fragment or variant thereof. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 12, 13, 14, or 15 is also disclosed.

A polypeptide that is a fragment or variant of the Sh1 protein including the amino acid sequence SEQ ID NO: 12, 13, 14, or 15, is also disclosed. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of 12, 13, 14, 15, is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, or 75 amino acids shorter than SEQ ID NO: 12, 13, 14, or 15.

Also disclosed are polynucleotides encoding the amino acid sequence SEQ ID NO: 12, 13, 14, 15, or fragments or variants thereof.

2. Non-Shattering Sh1 Polypeptides

An amino acid sequence encoding a non-shattering Sh1 gene product is also disclosed. Thus disclosed is a polypeptide encoded by the nucleic acid sequence of SEQ ID NO:7, 8, 9, 10, 11 or a fragment or variant thereof. Also disclosed is a polypeptide encoded by a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:7, 8, 9, 10, or 11. Also disclosed is a polypeptide encoded by a polynucleotide that hybridizes under stringent conditions to a polynucleotide consisting of the nucleic acid sequence SEQ ID NO: 7, 8, 9, 10, 11 or a fragment or variant thereof.

A polypeptide that is a fragment or variant of a non-shattering Sh1 gene product is also disclosed. Thus, a polypeptide encoded by a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of SEQ ID NO: 7, 8, 9, 10, 11 or a variant thereof is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, or more amino acids shorter than the polypeptide encoded by the nucleic acid sequence SEQ ID NO: 7, 8, 9, 10, or 11.

In a preferred embodiment, the non-shattering Sh1 gene product as it is found in S. bicolor includes the amino acid sequence of the polypeptide encoded by SEQ ID NO:7:

MPEDGYEWKK YGQKFIKNIQ KIRSYFRCRH KLCGAKKKVE
WHPRDPSGDL RIVYEGAHQH GAPAAAAPPG PGGQHHGGGA
SDFNRYELGA QYFGGAGRSH

(SEQ ID NO:16) or a variant thereof having one or more conservative amino acid substitutions and at least 90%, 95%, or more sequence identity compared to SEQ ID NO:16.

In another embodiment, the non-shattering Sh1 gene product as it is found in S. bicolor includes the amino acid sequence of the polypeptide encoded by SEQ ID NO:10:

MAEPGLEGSQ PVDLSKHPSG IVPTLQNIVS TVNLDCKLDL
KAIALQARNA EYNPKRFAAV IMRIREPKTT ALIFASGKMV
CTGAKSEQQS KLAARKYARI IQKLGFPAKF KDFKIQNIVG
SCDVKFPIRL EGLAYSHGAF SSYEPELFPG LIYRMKQPKI
VLLIFVSGKI VLTGAKVREE TYTAFENIYP VLTEFRKVQQ C

(SEQ ID NO:17) or a variant thereof having one or more conservative amino acid substitutions and at least 90%, 95%, or more sequence identity compared to SEQ ID NO:17.

Accordingly, in some embodiments, an amino acid sequence encoded by the Sh1 gene as it is found in S. bicolor includes the amino acid sequence of SEQ ID NO:16, or 17, or a fragment or variant thereof.

A polypeptide is therefore disclosed having the amino acid sequence SEQ ID NO: 16, or 17, or a fragment or variant thereof. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 16, or 17, or a fragment or variant thereof is also disclosed.

A polypeptide that is a fragment or variant of the Sh1 protein including the amino acid sequence SEQ ID NO: 16 or 17, is also disclosed. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of 16 or 17 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, or 75 amino acids shorter than SEQ ID NO: 16 or 17.

Also disclosed are polynucleotides encoding the amino acid sequence SEQ ID NO: 16 or 17, or fragments or variants thereof.

C. Functional Nucleic Acids

Also disclosed is a functional nucleic acid that silences Sh1 expression. The disclosed functional nucleic acid can in some embodiments also silence homologous seed shattering genes in other plants lacking a non-shattering variety. Thus, disclosed is functional nucleic acid that silences expression of a polynucleotide having the nucleic acid sequence SEQ ID NO:1, 2, 3, 4, 5, 6, 7, 8, 9, 10, 11, or fragments or variants thereof, or a polynucleotide encoding the polypeptide sequence SEQ ID NO: 12, 13, 14, 15, 16, 17, or fragments or variants thereof.

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with Sh1 mRNA or the genomic DNA of an Sh1 gene or they can interact with the polypeptide encoded by an Sh1 gene. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III—like enzyme, Dicer, into double stranded small interfering RNAs (siRNA) 21-23 nucleotides in length that contains 2 nucleotide overhangs on the 3′ ends (Elbashir, et al., Genes Dev., 15:188-200 (2001); Bernstein, et al., Nature, 409:363-6 (2001); Hammond, et al., Nature, 404:293-6 (2000)). In an ATP dependent step, the siRNAs become integrated into a multi-subunit protein complex, commonly known as the RNAi induced silencing complex (RISC), which guides the siRNAs to the target RNA sequence (Nykanen, et al., Cell, 107:309-21 (2001)). At some point the siRNA duplex unwinds, and it appears that the antisense strand remains bound to RISC and directs degradation of the complementary mRNA sequence by a combination of endo and exonucleases (Martinez, et al., Cell, 110:563-74 (2002)). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, et al., Nature, 411:494 498 (2001)) (Ui-Tei, et al., FEBS Lett 479:79-82 (2000)). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for an Sh1 gene.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-ITT™ inducible RNAi plasmid and lentivirus vectors. Disclosed herein are any shRNA designed as described above based on the sequences for the herein disclosed inflammatory mediators.

In some embodiments, the functional nucleic acid that silences expression of an Sh1 gene does so moderately. For example, methods of delaying seed shattering in plants using moderate dsRNA gene silencing is disclosed in U.S. Patent Publication 2006/0248612, which is incorporated by reference in its entirety.

Generally, moderate dsRNA gene silencing of genes involved in the development of the dehiscence zone and valve margins of fruits allows the isolation of transgenic lines with increased shatter resistance and reduced seed shattering, the fruits of which however may still be opened along the dehiscence zone by applying limited physical forces. This contrasts with transgenic plants wherein the dsRNA silencing is more pronounced, which can result in transgenic lines with indehiscent fruits, which no longer can be opened along the dehiscence zone, and which only open after applying significant physical forces by random breakage of the fruits, whereby the seeds remain predominantly within the remains of the fruits.

Moderate dsRNA gene silencing of genes can be conveniently achieved by operably linking the dsRNA coding DNA region to a relatively weak promoter region, or by choosing the sequence identity between the complementary sense and antisense part of the dsRNA encoding DNA region to be lower than 90% and preferably within a range of about 60% to 80%.

Thus, in one embodiment, a method is provided for reducing seed shattering in a plant by creating a population of transgenic lines of a plant, wherein the transgenic lines of the population exhibit variation in seed shatter resistance. This population may be obtained by introducing an expression vector into cells of a plant, to create transgenic cells, whereby the expression vector includes a plant-expressible promoter and a 3′ end region having transcription termination and polyadenylation signals functioning in cells of a plant, operably linked to a DNA region which when transcribed yields a double-stranded RNA molecule capable of reducing the expression of a gene endogenous to the plant, involved in the development of a dehiscence zone and valve margin of a fruit of the plant.

The RNA molecule can have a first (sense) RNA region and second (antisense) RNA region whereby the first RNA region includes a nucleotide sequence of at least 19 consecutive nucleotides having about 94% sequence identity to the nucleotide sequence of the endogenous gene; the second RNA region including a nucleotide sequence complementary to the at least 19 consecutive nucleotides of the first RNA region; the first and second RNA region being capable of base-pairing to form a double stranded RNA molecule between the at least 19 consecutive nucleotides of the first and second region.

Thus, in preferred embodiments, expression of a functional nucleic acid that silences expression of an Sh1 gene in plants increases seed shatter resistance compared to seed shatter resistance in an untransformed plant of the same species, while however maintaining an agronomically relevant threshability of the fruit. After regeneration of transgenic lines from the transgenic cells comprising the chimeric genes disclosed herein, a seed shatter resistant plant can be selected from the generated population.

D. Vectors and Constructs

Vectors and constructs containing an Sh1 gene, mRNA, cDNA, or variant or fragment thereof operably linked to an endogenous or heterologous expression control sequence are also provided. The constructs can include an expression cassette containing an Sh1 gene mRNA, cDNA, or variant or fragment thereof. For example, the expression constructs can include an expression cassette including a nucleic acid having the sequence SEQ ID NO:1, 2, 3, 4, 5, 6, 7, 8, 9, 10, 11, or fragments or variants thereof or a polynucleotide encoding a polypeptide having the amino acid sequence SEQ ID NO:12, 13, 14, 15, 16, 17, or fragments or variants thereof. The expression constructs can be used to control shattering in plants.

Also provided are vectors and constructs containing a nucleic acid sequence that silences Sh1 gene expression (e.g., RNAi) operably linked to an endogenous or heterologous expression control sequence. For example, the expression constructs can include an expression cassette that expresses a nucleic acid designed to inhibit or reduce expression of a nucleic acid having the sequence SEQ ID NO: SEQ ID NO:1, 2, 3, 4, 5, 6, 7, 8, 9, 10, 11, or fragments or variants thereof, or a polynucleotide encoding a polypeptide having the amino acid sequence SEQ ID NO:12, 13, 14, 15, 16, 17, or fragments or variants thereof.

Transformation constructs can be engineered such that transformation of the nuclear genome and expression of transgenes from the nuclear genome occurs. Alternatively, transformation constructs can be engineered such that transformation of the plastid genome and expression of the plastid genome Occurs.

An exemplary construct contains a nucleic acid sequence containing an Sh1 gene operatively linked in the 5′ to 3′ direction to a promoter that directs transcription of the nucleic acid sequence, and a 3′ polyadenylation signal sequence. In some embodiments, the encoded protein has at least about 10, 20, 30, 40, 50, 60, 70, 80, 90, or 100 percent gene shattering activity of the Sh1 gene in S. bicolor. In some embodiments the protein has at least about 10, 20, 30, 40, 50, 60, 70, 80, 90, or 100 percent gene shattering activity of the Sh1 gene in S. propinquum.

Another exemplary construct contains a nucleic acid sequence that silences Sh1 gene expression operatively linked in the 5′ to 3′ direction to a promoter that directs transcription of the nucleic acid sequence, and a 3′ polyadenylation signal sequence. In some embodiments, the transcribed nucleic acid sequence can result in at least about 10, 20, 30, 40, 50, 60, 70, 80, 90, or 100 percent inhibition of the Sh1 gene in S. propinquum. In some embodiments, the transcribed nucleic acid sequence can result in at least about 10, 20, 30, 40, 50, 60, 70, 80, 90, or 100 percent inhibition of the Sh1 gene in S. bicolor.

Generally, nucleic acid sequences containing an Sh1 gene are first assembled in expression cassettes behind a suitable promoter expressible in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors. Representative plant transformation vectors are described in plant transformation vector options available (Gene Transfer to Plants (1995), Potrykus, I. and Spangenberg, G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular biology-a laboratory course manual (1995), Maliga, P., Klessig, D. F., Cashmore, A. R., Gruissem, W. and Varner, J. E. eds. Cold Spring Laboratory Press, New York). An additional approach is to use a vector to specifically transform the plant plastid chromosome by homologous recombination (U.S. Pat. No. 5,545,818 to McBride, et al.), in which case it is possible to take advantage of the prokaryotic nature of the plastid genome and insert a number of transgenes as an operon.

In some embodiments the expression cassette includes endogenous 5′ untranslated sequence (5′ UTR), endogenou 3′ untranslated sequence (3′ UTR), or a combination thereof.

The following is a description of various components of typical expression cassettes.

1. Promoters

Plant promoters can be selected to control the expression of the transgene in different plant tissues or organelles, for all of which methods are known to those skilled in the art (Gasser & Fraley, Science 244:1293-99 (1989)). In a preferred embodiment, promoters are selected from those of plant or prokaryotic origin that are known to yield high expression in plastids. In certain embodiments the promoters are inducible. Inducible plant promoters are known in the art.

The transgenes can be inserted into an existing transcription unit (such as, but not limited to, psbA) to generate an operon. However, other insertion sites can be used to add additional expression units as well, such as existing transcription units and existing operons (e.g., atpE, accD). Such methods are described in, for example, U.S. Pat. App. Pub. 2004/0137631, which is incorporated herein by reference in its entirety. For an overview of other insertion sites used for integration of transgenes into the tobacco plastome, see Staub (Staub, J. M., “Expression of Recombinant Proteins via the Plastid Genome,” in: Vinci V A, Parekh S R (eds.) Handbook of Industrial Cell Culture: Mammalian, and Plant Cells, pp. 259-278, Humana Press Inc., Totowa, N.J. (2002)).

In general, the promoter can be from any class I, II or III gene. For example, any of the following plastidial promoters and/or transcription regulation elements can be used for expression in plastids. Sequences can be derived from the same species as that used for transformation. Alternatively, sequences can be derived from other species to decrease homology and to prevent homologous recombination with endogenous sequences.

For instance, the following plastidial promoters can be used for expression in plastids.

PrbcL promoter (Allison L A, Simon L D, Maliga P, EMBO J. 15:2802-2809 (1996); Shiina T, Allison L, Maliga P, Plant Cell 10:1713-1722 (1998));

PpsbA promoter (Agrawal G K, Kato H, Asayama M, Shirai M, Nucleic Acids Research 29:1835-1843 (2001));

Prrn 16 promoter (Svab Z, Maliga P, Proc. Natl. Acad. Sci. USA 90:913-917 (1993); Allison L A, Simon L D, Maliga P, EMBO J. 15:2802-2809 (1996));

PaccD promoter (Hajdukiewicz P T J, Allison L A, Maliga P, EMBO J. 16:4041-4048 (1997); WO 97/06250);

PclpP promoter (Hajdukiewicz P T J, Allison L A, Maliga P, EMBO J. 16:4041-4048 (1997); WO 99/46394);

PatpB, PatpI, PpsbB promoters (Hajdukiewicz P T J, Allison L A, Maliga P, EMBO J. 16:4041-4048 (1997));

PrpoB promoter (Liere K, Maliga P, EMBO J. 18:249-257 (1999));

PatpB/E promoter (Kapoor S, Suzuki J Y, Sugiura M, Plant J. 11:327-337 (1997)).

In addition, prokaryotic promoters (such as those from, e.g., E. coli or Synechocystis) or synthetic promoters can also be used.

Promoters vary in their strength, i.e., ability to promote transcription.

Depending upon the host cell system utilized, any one of a number of suitable promoters known in the art may be used. For example, for constitutive expression, the CaMV 35S promoter, the rice actin promoter, or the ubiquitin promoter may be used. For example, for regulatable expression, the chemically inducible PR-1 promoter from tobacco or Arabidopsis may be used (see, e.g., U.S. Pat. No. 5,689,044 to Ryals, et al.).

A suitable category of promoters is that which is wound inducible. Numerous promoters have been described which are expressed at wound sites. Preferred promoters of this kind include those described by Stanford, et al. Mol. Gen. Genet. 215:200-208 (1989), Xu, et al., Plant Molec. Biol. 22:573-588 (1993), Logemann, et al., Plant Cell, 1:151-158 (1989), Rohrmeier & Lehle, Plant Molec. Biol., 22: 783-792 (1993), Firek, et al., Plant Molec. Biol., 22:129-142 (1993), and Warner, et al., Plant J., 3: 191-201 (1993).

Suitable tissue specific expression patterns include green tissue specific, root specific, stem specific, and flower specific. Promoters suitable for expression in green tissue include many which regulate genes involved in photosynthesis, and many of these have been cloned from both monocotyledons and dicotyledons. A suitable promoter is the maize PEPC promoter from the phosphoenol carboxylase gene (Hudspeth & Grula, Plant Molec. Biol. 12:579-589 (1989)). A suitable promoter for root specific expression is that described by de Framond FEBS 290: 103-106 (1991); EP 0 452 269 to de Framond and a root-specific promoter is that from the T-1 gene. A suitable stem specific promoter is that described in U.S. Pat. No. 5,625,136 and which drives expression of the maize trpA gene.

The expression control sequence can be a dehiscence zone-selective regulatory element. The dehiscence zone-selective regulatory element can be from Sh1 or derived from a gene that is an ortholog of Sh1 and is selectively expressed in the valve margin or dehiscence zone of a seed plant. Dehiscence zone-selective regulatory elements also can be derived from a variety of other genes that are selectively expressed in the valve margin or dehiscence zone of a seed plant. For example, the rapeseed gene RDPG1 is selectively expressed in the dehiscence zone (Petersen, et al., Plant Mol. Biol., 31:517-527 (1996)). Thus, the RDPG1 promoter or an active fragment thereof can be a dehiscence zone-selective regulatory element as defined herein. Additional genes such as the rapeseed gene SAC51 also are known to be selectively expressed in the dehiscence zone; the SAC51 promoter or an active fragment thereof also can be a dehiscence zone-selective regulatory element (Coupe, et al., Plant Mol. Biol., 23:1223-1232 (1993)). The skilled artisan understands that a regulatory element of any such gene selectively expressed in cells of the valve margin or dehiscence zone can be a dehiscence zone-selective regulatory element.

Additional dehiscence zone-selective regulatory elements can be identified and isolated using routine methodology. Differential screening strategies using, for example, RNA prepared from the dehiscence zone and RNA prepared from adjacent fruit material can be used to isolate cDNAs selectively expressed in cells of the dehiscence zone (Coupe, et al., Plant Mol. Biol., 23:1223-1232 (1993)); subsequently, the corresponding genes are isolated using the cDNA sequence as a probe.

The promoter can be a relatively weak plant expressible promoter. Thus, the promoter can in some embodiments initiate and control transcription of the operably linked nucleic acids about 10 to about 100 times less efficient that an optimal CaMV35S promoter. Relatively weak plant expressible promoters include the promoters or promoter regions from the opine synthase genes of Agrobacterium spp. such as the promoter or promoter region of the nopaline synthase, the promoter or promoter region of the octopine synthase, the promoter or promoter region of the mannopine synthase, the promoter or promoter region of the agropine synthase and any plant expressible promoter with comparably activity in transcription initiation. Other relatively weak plant expressible promoters may be dehiscence zone selective promoters, or promoters expressed predominantly or selectively in dehiscence zone and/or valve margins of fruits, such as the promoters described in WO97/13865.

2. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

At the extreme 3′ end of the transcript, a polyadenylation signal can be engineered. A polyadenylation signal refers to any sequence that can result in polyadenylation of the mRNA in the nucleus prior to export of the mRNA to the cytosol, such as the 3′ region of nopaline synthase (Bevan, M., et al., Nucleic Acids Res., 11:369-385 (1983)).

3. Sequences for the Enhancement or Regulation of Expression

Numerous sequences have been found to enhance gene expression from within the transcriptional unit and these sequences can be used in conjunction with the genes to increase their expression in transgenic plants. For example, various intron sequences such as introns of the maize Adh1 gene have been shown to enhance expression, particularly in monocotyledonous cells. In addition, a number of non-translated leader sequences derived from viruses are also known to enhance expression, and these are particularly effective in dicotyledonous cells.

4. Coding Sequence Optimization

The coding sequence of the selected gene may be genetically engineered by altering the coding sequence for optimal expression in the crop species of interest. Methods for modifying coding sequences to achieve optimal expression in a particular crop species are well known (see, e.g. Perlak, et al., Proc. Natl. Acad. Sci. USA, 88:3324 (1991); and Koziel, et al, Biotechnol., 11: 94 (1993)).

5. Targeting Sequences

The disclosed vectors and constructs may further include, within the region that encodes the protein to be expressed, one or more nucleotide sequences encoding a targeting sequence. A “targeting” sequence is a nucleotide sequence that encodes an amino acid sequence or motif that directs the encoded protein to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The “targeting” sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof, such as a glyoxysome) an endoplasmic reticulum, an endosome, a vacuole, a plasma membrane, a cell wall, a mitochondria, a chloroplast or a plastid. A chloroplast targeting sequence is any peptide sequence that can target a protein to the chloroplasts or plastids, such as the transit peptide of the small subunit of the alfalfa ribulose-biphosphate carboxylase (Khoudi, et al., Gene, 197:343-351 (1997)). A peroxisomal targeting sequence refers to any peptide sequence, either N-terminal, internal, or C-terminal, that can target a protein to the peroxisomes, such as the plant C-terminal targeting tripeptide SKL (Banjoko, A. & Trelease, R. N. Plant Physiol., 107:1201-1208 (1995); T. P. Wallace et al., “Plant Organellular Targeting Sequences,” in Plant Molecular Biology, Ed. R. Croy, BIOS Scientific Publishers Limited (1993) pp. 287-288, and peroxisomal targeting in plant is shown in M. Volokita, The Plant J., 361-366 (1991)).

Plastid targeting sequences are known in the art and include the chloroplast small subunit of ribulose-1,5-bisphosphate carboxylase (Rubisco) (de Castro Silva Filho, et al., Plant Mol. Biol., 30:769-780 (1996); Schnell, et al., J. Biol. Chem. 266(5):3335-3342 (1991)); 5-(enolpyruvyl)shikimate-3-phosphate synthase (EPSPS) (Archer, et al., J. Bioenerg. Biomemb., 22(6):789-810 (1990)); tryptophan synthase (Zhao, et al., J. Biol. Chem., 270(11):6081-6087 (1995)); plastocyanin (Lawrence, et al., J. Biol. Chem., 272(33):20357-20363 (1997)); chorismate synthase (Schmidt, et al., J. Biol. Chem., 268(36):27447-27457 (1993)); and the light harvesting chlorophyll a/b binding protein (LHBP) (Lamppa, et al., J. Biol. Chem. 263:14996-14999 (1988)). See also Von Heijne, et al., Plant Mol. Biol. Rep., 9:104-126 (1991); Clark, et al., J. Biol. Chem., 264:17544-17550 (1989); Della-Cioppa, et al., Plant Physiol., 84:965-968 (1987); Romer, et al., Biochem. Biophys. Res. Commun., 196:1414-1421 (1993); and Shah, et al., Science, 233:478-481 (1986). Alternative plastid targeting signals have also been described in the following: US 2008/0263728; Miras, et al., J Biol Chem, 277(49): 47770-8 (2002); Miras, et al., J Biol Chem, 282: 29482-29492 (2007)).

E. Plants and Tissues for Transfection

Both dicotyledons (“dicots”) and monocotyledons (“monocots”) can be used in the disclosed positive selection system. Monocot seedlings typically have one cotyledon (seed-leaf), in contrast to the two cotyledons typical of dicots. Eudicots are dicots whose pollen has three apertures (i.e. triaperturate pollen), through one of which the pollen tube emerges during pollination. Eudicots contrast with the so-called ‘primitive’ dicots, such as the magnolia family, which have uniaperturate pollen (i.e. with a single aperture).

Monocots include one of the large divisions of Angiosperm plants (flowering plants with seeds protected within a vessel). They are herbaceous plants with parallel veined leaves and have an embryo with a single cotyledon, as opposed to dicot plants (dicotyledonous), which have an embryo with two cotyledons. Most of the important staple crops of the world, the so-called cereals, such as wheat, barley, rice, maize, sorghum, oats, rye and millet, are monocots. Thus, the plant can be a grass, such as wheat, barley, rice, maize, sorghum, oats, rye and millet.

Thus, the plant can be a cereal crop such as wheat, oat, barley, or rice; a forage such as bahiagrass, dallisgrass, kleingrass, guineagrass, reed canarygrass, orchardgrass, ricegrass, foxtail, or vetch; a legume such as soybean, lentil, or chickpea; an oilseed such as canola; a vegetable such as onion or carrot; or a specialty crop such as caraway, hemp, or sesame.

In some embodiments, the plant is a sorghum. Thus, the plant can be of the species Sorghum almum, Sorghum amplum, Sorghum angustum, Sorghum arundinaceum, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum purpureosericeum, Sorghum stipoideum, Sorghum timorense, Sorghum trichocladum, Sorghum versicolor, Sorghum virgatum, and Sorghum vulgare

In some embodiments, the plant is a miscanthus. Thus, the plant can be of the species Miscanthus floridulus, Miscanthus giganteus, Miscanthus sacchariflorus (Amur silver-grass), Miscanthus sinensis, Miscanthus tinctorius, or Miscanthus transmorrisonensis.

Additional representative plants useful in the compositions and methods disclosed herein include the Brassica family including napus, rapa, oleracea, nigra, carinata and juncea; industrial oilseeds such as Camelina sativa, Crambe, Jatropha, castor; Arabidopsis thaliana; soybean; cottonseed; sunflower; palm; coconut; rice; safflower; peanut; mustards including Sinapis alba; sugarcane and flax.

Crops harvested as biomass, such as silage corn, alfalfa, switchgrass, or tobacco, also are useful with the methods disclosed herein. Representative tissues for transformation using these vectors include protoplasts, cells, callus tissue, leaf discs, pollen, and meristems.

### III. Methods of Modulating Seed Shattering

A. Methods of Reducing, Inhibiting, Delaying, or Eliminating Shattering

Seed/grain losses due to shattering remain a significant economic problem in common cereal crops such as wheat, oat, barley, and rice; forages such as bahiagrass, dallisgrass, kleingrass, guineagrass, reed canarygrass, orchardgrass, ricegrass, foxtail, and vetch; legumes such as soybean, lentil, and chickpea; oilseeds such as canola; vegetables such as onion and carrot; and specialty crops such as caraway, hemp, and sesame. Moreover, economical large-scale cultivation of many prospective new crops would be greatly facilitated by suppression of shattering—some examples include wild rice, birdsfoot trefoil, castor, oilseed spurge, Veronica and others.

Methods for reducing, inhibiting, delaying or eliminating shattering in a plant including, but not limited to a sorghum plant, are disclosed. As discussed in more detail in the Examples below, it is believed that the gene that conveys a shattering phenotype in sorghum is dominant to the gene the conveys a non-shattering phenotype, because following a cross of non-shattering S. bicolor with the shattering S. propinquum, all F1 progenies shattered. Accordingly, it is believed that reducing the expression levels of a gene product from a gene that conveys a shattering phenotype, increasing the expression levels of a gene product from a gene that conveys a non-shattering phenotype, or combinations thereof can reduce, inhibit, delay or eliminate shattering in a plant that is typically a shattering plant.

For example, a method of reducing, inhibiting, delaying or eliminating fruit dehiscence in a plant is provided, involving introducing to the plant a nucleic acid sequence that suppresses the expression of an endogenous gene orthologous to sorghum grain shattering gene (Sh1) that conveys a shattering phenotype. In some embodiments, inhibiting or reducing expression of the Sh1 gene, mRNA, a polypeptide encoded thereby, or variants thereof from Sorghum propinquum, including transient inhibition or reduction in expression can reduce, inhibit, delay, or inhibit shattering. Thus, the methods can involve introducing to the plant a composition that inhibits activity of the shattering gene (Sh1) from a Sorghum propinquum plant, or a variant thereof that conveys a shattering phenotype.

Thus, the methods can involve introducing to the plant a composition including a polynucleotide having a nucleic acid sequence that silences expression of a polynucleotide having a nucleic acid sequence SEQ ID NO:1, 2, 3, 4, 5, or 6 or fragments or variants thereof, or a polynucleotide encoding the polypeptide sequence SEQ ID NO:12, 13, 14, or 15, or fragments or variants thereof. As a result of this method, the transgenic plant preferably has reduced seed shattering compared to non-transgenic (e.g., wild-type) plant of the same species. Preferably, the transgenic plant retains agronomically relevant threshability.

A method of reducing, inhibiting, delaying or eliminating fruit dehiscence in a plant is also provided, involving introducing to the plant a composition that increases or promotes the expression of an endogenous gene orthologous to sorghum grain shattering gene (Sh1) that conveys a non-shattering phenotype. In some embodiments, increasing or promoting expression of the Sh1 gene, mRNA, a polypeptide encoded thereby, or variants thereof from Sorghum bicolor, including a transient increase or promotion in expression can reduce, inhibit, delay, or eliminate shattering. Thus, the methods can involve introducing to the plant a composition that promotes activity of the shattering gene (Sh1) from a Sorghum bicolor plant.

Thus, the methods can involve introducing to the plant a nucleic acid sequence that promotes expression of a polynucleotide having a nucleic acid sequence SEQ ID NO:7, 8, 9, 10, 11, or fragments of variants therefore or a polynucleotide encoding the polypeptide sequence SEQ ID NO: 16 or 17, or fragments or variants thereof. As a result of this method, the transgenic plant preferably has accelerated seed shattering compared to non-transgenic (e.g., wild-type) plant of the same species. Preferably, the transgenic plant retains agronomically relevant threshability.

In some embodiments, the methods can involve introducing to the plant a composition that inhibits activity of the shattering gene (Sh1) from a Sorghum propinquum plant and introducing to the plant a composition that promotes activity of the shattering gene (Sh1) from a Sorghum bicolor plant.

B. Methods of Promoting, Increasing, or Accelerating Shattering

Shattering also contributes to the dissemination of agricultural weeds such as Johnson grass, wild oat, proso millet, and red rice. If premature shattering could be induced it could cause dispersal before seeds are viable, reducing the weed “seed reservoir” in the soil.

Methods for promoting, increasing, or accelerating shattering in a plant including, but not limited to a sorghum plant, are disclosed. As discussed above, it is believed that the gene that conveys a shattering phenotype in sorghum is dominant to the gene that conveys a non-shattering phenotype. Accordingly, it is believed that increasing the expression levels of a gene product from a gene that conveys a shattering phenotype, decreasing the expression levels of a gene product from a gene that conveys a non-shattering phenotype, or combinations thereof can promote, increase, or accelerate shattering in a plant that is typically a non-shattering plant.

For example, a method of promoting, increasing, or accelerating shattering fruit dehiscence in a plant is provided, involving introducing to the plant a nucleic acid sequence that suppresses the expression of an endogenous gene orthologous to sorghum grain shattering gene (Sh1) that conveys a non-shattering phenotype. In some embodiments, inhibiting or reducing expression of the Sh1 gene, mRNA, a polypeptide encoded thereby, or variants thereof from Sorghum bicolor, including transient inhibition or reduction in expression can promote, increase, or accelerate shattering. Thus, the methods can involve introducing to the plant a composition that inhibits activity of the shattering gene (Sh1) from a Sorghum bicolor plant.

Thus, the methods can involve introducing to the plant a composition including a polynucleotide having a nucleic acid sequence that silences expression of a polynucleotide having a nucleic acid sequence SEQ ID NO:7, 8, 9, 10, 11, or fragments of variants therefore or a polynucleotide encoding the polypeptide sequence SEQ ID NO: 16 or 17, or fragments or variants thereof. As a result of this method, the transgenic plant preferably has increased or accelerated seed shattering compared to non-transgenic (e.g., wild-type) plant of the same species.

A method of promoting, increasing, or accelerating shattering fruit dehiscence in a plant is also provided, involving introducing to the plant a composition that increases or promotes the expression of an endogenous gene orthologous to sorghum grain shattering gene (Sh1) that conveys a shattering phenotype. In some embodiments, increasing or promoting expression of the Sh1 gene, mRNA, a polypeptide encoded thereby, or variants thereof from Sorghum propinquum, including a transient increase or promotion in expression can reduce, inhibit, delay, or inhibit shattering. Thus, the methods can involve introducing to the plant a composition that promotes activity of the shattering gene (Sh1) from a Sorghum propinquum plant.

Thus, the methods can involve introducing to the plant a nucleic acid sequence that promotes expression of a polynucleotide having a nucleic acid sequence SEQ ID NO:1, 2, 3, 4, 5, or 6 or fragments or variants thereof, or a polynucleotide encoding the polypeptide sequence SEQ ID NO:12, 13, 14, or 15, or fragments or variants thereof. As a result of this method, the transgenic plant preferably has accelerated seed shattering compared to non-transgenic (e.g., wild-type) plant of the same species.

In some embodiments, the methods can involve introducing to the plant a composition that inhibits activity of the shattering gene (Sh1) from a Sorghum bicolor plant and introducing to the plant a composition that promotes activity of the shattering gene (Sh1) from a Sorghum propinquum plant.

C. Methods of Altering Lignin Deposition Around the Seed-Stalk Interface

Towards the end of the floral development in the beginning of the shattering process, there is significant lignin deposition at the seed-stalk interface. The lignification of those tissues is part of the programmed cell death and facilitates the break-off of the seeds from the stalk. It has been discovered that the gene that controls shattering in sorghum also controls lignin deposition around the seed-stalk interface. Accordingly, the methods described above for decreasing or delaying shattering can also be used to decrease lignin deposition at the seed-stalk interface and around the shattering zone of a plant, and the methods described above for increasing or accelerating shattering can also be used to increase lignin deposition at the seed-stalk interface and around the shattering zone of plant.

### IV. Methods of Making Transgenic Plants

A. Plant Transformation Techniques

The transformation of suitable agronomic plant hosts using vectors expressing transgenes can be accomplished with a variety of methods and plant tissues. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765 to Coffee, et al; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga, et al. eds.) Cold Spring Laboratory Press, New York (1995)).

Soybean can be transformed by a number of reported procedures (U.S. Pat. Nos. 5,015,580 to Christou, et al; 5,015,944 to Bubash; 5,024,944 to Collins, et al; 5,322,783 to Tomes, et al; 5,416,011 to Hinchee, et al; 5,169,770 to Chee, et al.).

A number of transformation procedures have been reported for the production of transgenic maize plants including pollen transformation (U.S. Pat. No. 5,629,183 to Saunders, et al.), silicon fiber-mediated transformation (U.S. Pat. No. 5,464,765 to Coffee, et al.), electroporation of protoplasts (U.S. Pat. Nos. 5,231,019 Paszkowski, et al; 5,472,869 to Krzyzek, et al; 5,384,253 to Krzyzek, et al.), gene gun (U.S. Pat. Nos. 5,538,877 to Lundquist, et al. and 5,538,880 to Lundquist, et al.), and Agrobacterium-mediated transformation (EP 0 604 662 A1 and WO 94/00977 both to Hiei Yukou, et al.). The Agrobacterium-mediated procedure is particularly preferred as single integration events of the transgene constructs are more readily obtained using this procedure which greatly facilitates subsequent plant breeding. Cotton can be transformed by particle bombardment (U.S. Pat. Nos. 5,004,863 to Umbeck and 5,159,135 to Umbeck). Sunflower can be transformed using a combination of particle bombardment and Agrobacterium infection (EP 0 486 233 A2 to Bidney, Dennis; U.S. Pat. No. 5,030,572 to Power, et al.). Flax can be transformed by either particle bombardment or Agrobacterium-mediated transformation. Switchgrass can be transformed using either biolistic or Agrobacterium mediated methods (Richards, et al., Plant Cell Rep. 20:48-54 (2001); Somleva, et al., Crop Science, 42:2080-2087 (2002)). Methods for sugarcane transformation have also been described (Franks & Birch Aust. J. Plant Physiol. 18, 471-480 (1991); WO 2002/037951 to Elliott, Adrian, Ross, et al.).

Methods for transformation of sorghum are known and disclosed, for example, in Able, et al. (2001). In Vitro Cellular & Developmental Biology-Plant 37:341-348; Battraw, et al. (1991). Theoretical and Applied Genetics 82:161-168; Carvalho, C. H. S., et al. 2004. Genetics and Molecular Biology 27:259-269; Casas, A. M., et al. 1997. In Vitro Cellular & Developmental Biology-Plant 33:92-100; Casas, A. M., et al. 1993. Proc Nat. Acad. Sci. U.S.A. 90:11212-11216; Devi, P. B., et al. 2003. Plant Biosystems 137:249-254; Gao, Z.S2005a. Plant Biotechnology Journal 3:591-599; Gao, Z. S., et al. 2005b. Genome 48:321-333; Gray, S. J., et al. 2004. Sorghum Tissue Culture and Transformation:35-43; Hagio, T., et al. 1991. Plant Cell Reports 10:260-264; Howe, A., et al. 2006. Plant Cell Reports 25:784-791; Jeoung, J. M., et al. 2002. Hereditas 137:20-28; Jeoung, J. M., et al. 2004. Sorghum Tissue Culture and Transformation:57-64; Krishnaven, S., et al. 2004. Sorghum Tissue Culture and Transformation:65-74; Nguyen, T. V., et al. 2007. Plant Cell Tissue and Organ Culture 91:155-164; Park, S. H., et al. 1998. Cell Biology—a Laboaratory Handbook, 2nd Edition, Vol 4:176-182; Rao, S. V., et al. 2004. Sorghum Tissue Culture and Transformation:45-50; Rathus, C., et al. 2004. Sorghum Tissue Culture and Transformation:25-34; Sai, N. S., et al. 2006. Plant Cell Reports 25:174-182; Seetharama, N., et al. Plant Cell Tissue and Organ Culture 61:169-173; Shrawat, A. K., et al. 2006. Plant Biotechnology Journal 4:575-603; Tadesse, Y., et al. 2003. Plant Cell Tissue and Organ Culture 75:1-18; Wang, W. Q., et al. 2007. Biotechnology and Applied Biochemistry 48:79-83; Williams, S. B., et al. 2004. Transgenic Crops of the World: Essential Protocols:89-102; Zhao, Z., et al. 2003. Genetic Transformation of Plants 23:91-107; Zhao, Z.Y. 2006. Agrobacterium Protocols, Second Edition, Vol 1 343:233-244; Zhao, Z. Y., et al. 2000. Plant Molecular Biology 44:789-798; Zhong, H., et al. 1998. Journal of Plant Physiology 153:719-726.

Recombinase technologies which are useful in practicing the current invention include the cre-lox, FLP/FRT and Gin systems. Methods by which these technologies can be used for the purpose described herein are described for example in (U.S. Pat. No. 5,527,695 to Hodges et al; Dale and Ow, Proc. Natl. Acad. Sci. USA, 88:10558-10562 (1991); Medberry et al., Nucleic Acids Res., 23: 485-490 (1995)).

Engineered minichromosomes can also be used to express one or more genes in plant cells. Cloned telomeric repeats introduced into cells may truncate the distal portion of a chromosome by the formation of a new telomere at the integration site. Using this method, a vector for gene transfer can be prepared by trimming off the arms of a natural plant chromosome and adding an insertion site for large inserts (Yu et al., Proc Natl Acad Sci USA, 103:17331-6 (2006); Yu et al., Proc Natl Acad Sci USA, 104:8924-9 (2007)). The utility of engineered minichromosome platforms has been shown using Cre/lox and FRT/FLP site-specific recombination systems on a maize minichromosome where the ability to undergo recombination was demonstrated (Yu et al., Proc Natl Acad Sci USA, 103:17331-6 (2006); Yu et al., Proc Natl Acad Sci U S A, 104:8924-9 (2007)). Such technologies could be applied to minichromosomes, for example, to add genes to an engineered plant. Site specific recombination systems have also been demonstrated to be valuable tools for marker gene removal (Kerbach, S. et al., Theor. Appl. Genet. 111:1608-1616 (2005)), gene targeting (Chawla, R. et al., Plant Biotechnol J., 4:209-218 (2006); Choi, S. et al., Nucleic Acids Res., 28, E19 (2000); Srivastava V & Ow D W, Plant Mol. Biol. 46:561-566 (2001); Lyznik L A et al., Nucleic Acids Res., 21: 969-975 (1993)) and gene conversion (Djukanovic V et al., Plant Biotechnol J., 4:345-357 (2006).

An alternative approach to chromosome engineering in plants involves in vivo assembly of autonomous plant minichromosomes (Carlson et al., PLoS Genet., 3:1965-74 (2007). Plant cells can be transformed with centromeric sequences and screened for plants that have assembled autonomous chromosomes de novo. Useful constructs combine a selectable marker gene with genomic DNA fragments containing centromeric satellite and retroelement sequences and/or other repeats.

Another approach useful to the described invention is Engineered Trait Loci (“ETL”) technology (U.S. Pat. No. 6,077,697; US Patent Application 2006/0143732). This system targets DNA to a heterochromatic region of plant chromosomes, such as the pericentric heterochromatin, in the short arm of acrocentric chromosomes. Targeting sequences may include ribosomal DNA (rDNA) or lambda phage DNA. The pericentric rDNA region supports stable insertion, low recombination, and high levels of gene expression. This technology is also useful for stacking of multiple traits in a plant (US Patent Application 2006/0246586).

Zinc-finger nucleases (ZFNs) are also useful for practicing the invention in that they allow double strand DNA cleavage at specific sites in plant chromosomes such that targeted gene insertion or deletion can be performed (Shukla et al., Nature, (2009); Townsend et al., Nature, (2009).

Following transformation by any one of the methods described above, the following procedures can, for example, be used to obtain a transformed plant expressing the transgenes: select the plant cells that have been transformed on a selective medium, regenerate the plant cells that have been transformed to produce differentiated plants, select transformed plants expressing the transgene producing the desired level of desired polypeptide(s) in the desired tissue and cellular location.

Transformation techniques for dicotyledons are well known in the art and include Agrobacterium-based techniques and techniques that do not require Agrobacterium. Non-Agrobacterium techniques involve the uptake of heterologous genetic material directly by protoplasts or cells. This is accomplished by PEG or electroporation mediated uptake, particle bombardment-mediated delivery, or microinjection. In each case the transformed cells may be regenerated to whole plants using standard techniques known in the art.

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

Plants from transformation events are grown, propagated and bred to yield progeny with the desired trait, and seeds are obtained with the desired trait, using processes well known in the art.

B. Plastid Transformation

In another embodiment the transgene is directly transformed into the plastid genome. Plastid transformation technology is extensively described in U.S. Pat. Nos. 5,451,513 to Maliga et al., 5,545,817 to McBride et al., and 5,545,818 to McBride et al., in PCT application no. WO 95/16783 to McBride et al., and in McBride et al. Proc. Natl. Acad. Sci. USA 91, 7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Suitable plastids that can be transfected include, but are not limited to, chloroplasts, etioplasts, chromoplasts, leucoplasts, amyloplasts, proplastids, statoliths, elaioplasts, proteinoplasts and combinations thereof.

### V. Screening Methods

Methods are also provided for identifying chemical treatments that can modify natural seed dispersal.

In some embodiments, the method involves administering a candidate agent to a transgenic plant disclosed herein and comparing the effect of the administration on seed shattering in the plant to a control. For example, the purpose of the method can be to identify a candidate agent that causes the transgenic plant to shatter prematurely. For example, it would be desirable to identify an agent the causes weeds to disseminate its seeds before they are mature. Alternatively, the purpose of the method can be to identify a candidate agent that causes the transgenic plant to delay seed shatter.

In some embodiments, the method involves contacting cells expressing an Sh1 gene disclosed herein with a candidate agent, monitoring the effect of the candidate agent on Sh1 gene expression, and comparing the effect of the candidate agent on Sh1 gene expression to a control. For example, the purpose of the method can be to identify an agent that promotes Sh1 gene expression of an Sh1 gene that conveys a shattering phenotype. For example, in some embodiments, the agent promotes expression of SEQ ID NO:1, 2, 3, 4, 5, or 6 or fragments or variants thereof, or a polynucleotide encoding the polypeptide sequence SEQ ID NO:12, 13, 14, or 15, or fragments or variants thereof. In another embodiment, the method can be to identify an agent that reduces or inhibits Sh1 gene expression of an Sh1 gene that conveys a non-shattering phenotype. For example, in some embodiments, the agent reduces or inhibits expression of SEQ ID NO:7, 8, 9, 10, or 11 or fragments or variants thereof, or a polynucleotide encoding the polypeptide sequence SEQ ID NO:16, or 17 or fragments or variants thereof.

In some embodiments, the purpose of the method can be to identify an agent that could be used to promote Sh1 gene expression of an Sh1 gene that conveys a non-shattering phenotype. For example, in some embodiments, the agent promotes expression of SEQ ID NO:7, 8, 9, 10, or 11 or fragments or variants thereof, or a polynucleotide encoding the polypeptide sequence SEQ ID NO:16, or 17 or fragments or variants thereof. Alternatively, the purpose of the method can be to identify an agent that inhibits gene expression of an Sh1 gene that conveys a shattering phenotype. For example, in some embodiments, the agent reduces or inhibits expression of SEQ ID NO:1, 2, 3, 4, 5, or 6 or fragments or variants thereof, or a polynucleotide encoding the polypeptide sequence SEQ ID NO:12, 13, 14, or 15, or fragments or variants thereof.

The effect of the agent can be compared to control. For example, in some embodiments, the expression of a Sh1 gene or gene product in a plant treated with the agent is compared to the expression of a Sh1 gene or gene product in a plant that is not treated with the agent. In some embodiments, the agent conveys a non-shattering phenotype to a plant that exhibits a shattering phenotype in the absence of the agent. In other embodiments, the agent conveys a shattering phenotype to a plant that exhibits a non-shattering phenotype in the absence of the agent.

Methods of determining gene or protein expression levels are known in the art. For example, mRNA levels can be determined using assays such as RT-PCT or gene array assays. Protein expression can be detected using routine methods, such as immunodetection methods. The methods can be cell-based or cell-free assays. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the disclosed screening procedure. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds.

Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have a desired activity, further fractionation of the positive lead can be used to isolate chemical constituents responsible for the observed effect. Thus, the goal of the extraction, fractionation, and purification process is the careful characterization and identification of a chemical entity within the crude extract having the activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

### VI. Methods of Identifying Shattering Genes in Related Plants

Methods are also provided for identifying genes that regulate the seed shattering process in other plants. In preferred embodiments, the plant is closely related to Sorghum propinquum. Thus, in some embodiments, the plant is Sorghum halepense, Miscanthus, or Saccharum.

In some embodiments, the method involves scanning the genetic sequences of a plant for genes that are homologous to Sh1. In this way, naturally occurring variants of the Sh1 gene can be identified and the phenotype associated with that variant can be analyzed. In one embodiment, mutations in the Sh1 homolog that prevent shattering are identified. The plants containing a mutated gene from a Sh1 homolog are then crossed using standard breeding techniques to obtain plants homozygous for the Sh1 mutation and do not shatter seeds. Preferred plants for identifying mutated Sh1 homologs include heterozygous polyploids such as sugarcane and Miscanthus.

In still another embodiment Sh1 homologs are identified in plants and mutated to produce a non-shattering plant.

In some embodiments, an Sh1 homolog gene product that conveys a non-shattering phenotype has a deletion of the about 44 N-terminal amino acids relative to SEQ ID NO:12. Accordingly, in some embodiments, an Sh1 homolog that conveys a non-shattering phenotype has nucleic acid sequence of SEQ ID NO:7, 8, 9, or 11, or an amino acids sequence of SEQ ID NO:16.

In some embodiments, an Sh1 homolog gene product that conveys a shattering phenotype includes about 44 N-terminal amino acids of SEQ ID NO:12. Accordingly, in some embodiments, an Sh1 homolog that conveys a non-shattering phenotype has nucleic acid sequence of SEQ ID NO:1, 2, 3, or 5, or an amino acids sequence of SEQ ID NO:12, 14, or 15.

### VII. Methods of Identifying Molecular Interactions

Methods are provided for identifying molecular interactions such as nucleic acid-protein and protein-protein interactions. In some embodiments, the molecular interaction regulates gene or protein expression of Sh1, or Sh1 protein activity. For example, the disclosed sequences can be used as the target, or bait sequence to identify nucleic acid-protein interactions using methods including, but not limited to, electrophoretic mobility shift assays (“gel shift” assays), yeast one-hybrid screens, chromatin immunoprecipitation-sequencing (also known as ChIP-Sequencing or ChIP-Seq). In one embodiment, DNA-binding proteins that bind within or adjacent to the Sh1 gene are identified. In another embodiment, Sh1 regulatory or expression sequences within or adjacent to the Sh1 gene are identified.

In some embodiments Sh1 regulates the expression or activity of another gene or protein. Sh1 protein can be used as a probe to identify nucleic acid or protein binding partners using methods including, but not limited to, electrophoretic mobility shift assays (“gel shift” assays), ChIP-Seq, yeast one-hybrid, and yeast two-hybrid screens. In one embodiment, nucleic acid sequences bound by Sh1 protein are identified. In another embodiment, proteins that bind to Sh1 protein are identified.

In some embodiments Sh1 is the subject of microarray or gene chip analysis. Oligonucleotide or cDNA microarray can be used to profile gene expression and identify mutations such as single nucleotide polymorphisms. For example, microarray analysis can be used to compare Sh1 expression in different species or organisms, to monitor Sh1 expression under different physiological or molecular conditions, or to identify genes that are regulated by Sh1 expression.

### EXAMPLES

### Example 1

### Genetic Mapping of the Sh1 Locus in S. bicolor×S. propinquum F2 Population

Substitution mapping (Paterson, et al., Genetics, 124(3):735-42 (1990)) was used for the genetic mapping of the chromosome segment associated with Sh1. In the cross S. bicolor×S. propinquum, all F1 progenies shattered, indicating that Sh1 was completely dominant (Paterson, et al., Science, 269: 1714-18 (1995)). The mapping population was comprised of 370 F2 individuals (740 informative gametes). DNA markers that were mapped directly or inferred by comparative data to locate close to Sh1 were applied to a panel of recombinants in the region. The markers that flanked, or co-segregated with the shattering trait were identified.

### Example 2

### Sequencing, Assembly and Annotation of S. propinquum BACs

An S. propinquum bacterial artificial chromosome (BAC) library with high coverage of the genome (Lin, et al., Molecular Breeding, 5: 511-520 (1999)) was screened with the DNA markers closely linked to Sh1. BACs that hybridized to the two flanking genetic markers in the shattering region were fingerprinted via restriction enzyme digestion, and used to construct physical contigs (Soderlund, et al., Cabios, 13: 523-535 (1997)). One contig that spans the entire length between the two flanking markers was constructed. Several BACs forming a tiling path of the contig were selected. The DNA of the BACs was isolated, sheared, end-repaired into subclones and Sanger-sequenced.

TABLE 1
Assembly status of the S. propinquum BACs around the
putative shattering region.

| BAC ID | # of scaffolds | # of contigs | Size | Total # of reads |
|---|---|---|---|---|
| YRL39E21 | 4 | 5 | 226 kb | 5898 |
| YRL07C13 | 1 | 3 | 111 kb | 2118 |
| YRL62I16 | 6 | 15 | 120 kb | 2304 |
| YRL38P22 | 5 | 16 | 210 kb | 3355 |
| YRL20H16 | 3 | 5 | 61 kb | 1772 |
| YRL58G20 | 3 | 9 | 115 kb | 3840 |
| YRL69G23 | 2 | 12 | 157 kb | 3137 |
| YRL34P18 | 3 | 4 | 55 kb | 1536 |
| YRL79E08 | 5 | 26 | 119 kb | 2304 |
| YRL60N05 | 3 | 14 | 142 kb | 2131 |

Only contigs that are >1 kb length were counted.

Sequence assembly followed the PHRED/PHRAP/CONSED pipeline (Ewing, et al., Genome Research, 8:175-85 (1998)). Alternative assemblies were also attempted with the TIGR and CELERA assemblers but PHRAP was chosen because it shows the lowest error rate among the three programs. Thus far, draft assemblies were obtained for the 10 BACs containing un-finished contigs within each BAC (Table 1). Finally, the reads from the 10 overlapping BACs were pooled and assembled into 108 contigs, comprising a total size of 1.06 Mb of the entire region in S. propinquum.

Gene structures in the S. propinquum shattering region were predicted using the similarity-based gene prediction software GENEWISE, using the S. bicolor predicted genes (Sbi version 1.4) as the reference sequences. GENEWISE predicted 95 S. propinquum gene models (with a median size of 906 base pairs), corresponding to 95 S. bicolor gene models. A total of 80 genes are within the boundary of the two flanking markers in the linkage mapping.

Comparative analyses between S. bicolor and S. propinquum orthologs show that they are similar at the DNA level. For the 95 gene loci predicted, 9 loci show no protein changes between the two species. The median of synonymous substitution per synonymous site (Ks) is 0.0215 in the shattering region. This median Ks value corresponds to ˜1.7 million years of divergence between S. propinquum and S. bicolor, using a rate estimate of 6.5×10⁻⁹ synonymous substitutions per year (Gaut, et al., Proc Natl Acad Sci USA, 93: 10274-79 (1996)). Median non-synonymous substitution value (Ka) is 0.0063 between the two species. Most genes show Ka/Ks ratio less than 1, indicating purifying selection (Yang, et al., Trends Ecol Evol, 15:496-503 (2000)). Surprisingly, 10 genes among the 95 genes have a Ka/Ks ratio greater than 1 (FIG. 1), which is often interpreted as evidence supporting positive selection (Yang, et al., Trends Ecol Evol, 15:496-503 (2000)). However, since all 10 genes with high Ka/Ks ratio only have putative function, it is possible that some genes or some parts of the genes might be results of mis-annotations.

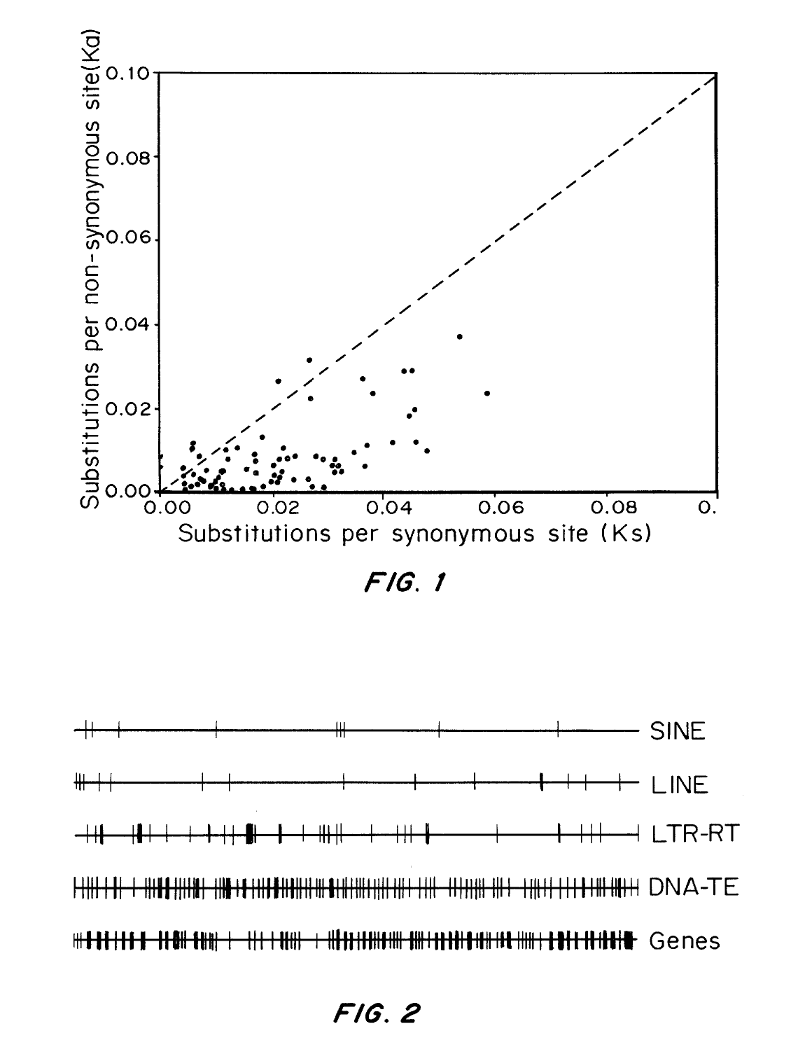

Repeats within the shattering region of the two sorghum species were identified using REPEATMASKER version 3.2 (Huda, et al., Methods Mol Biol, 537:323-36 (2009)). The physical positions of these elements in S. bicolor are shown in FIG. 2. The overall repeat level is comparable between the two sorghum species in this region. There is a higher level of retroelements in S. propinquum (7.7%) than in S. bicolor (4.9%). Previous study found that the entire sorghum genome contains 55% retrotransposons, with preferential insertions of these elements in the heterochromatic regions (Paterson, et al., Nature, 457:551-56 (2009)). Therefore, the relatively low percentage of retroelements observed in this region compared to the genome average is consistent with features of euchromatin. Contrary to the relative abundance of retroelements, there are slightly more DNA transposons in S. bicolor (8.5%) than in S. propinquum (7.3%). The most abundant type of retroelement and DNA transposon in this region in both sorghum species are Gypsy/DIRS1 and Tourist/Harbinger, respectively.

### Example 2 S. propinquum BACs Align to an Orthologous S. bicolor Region

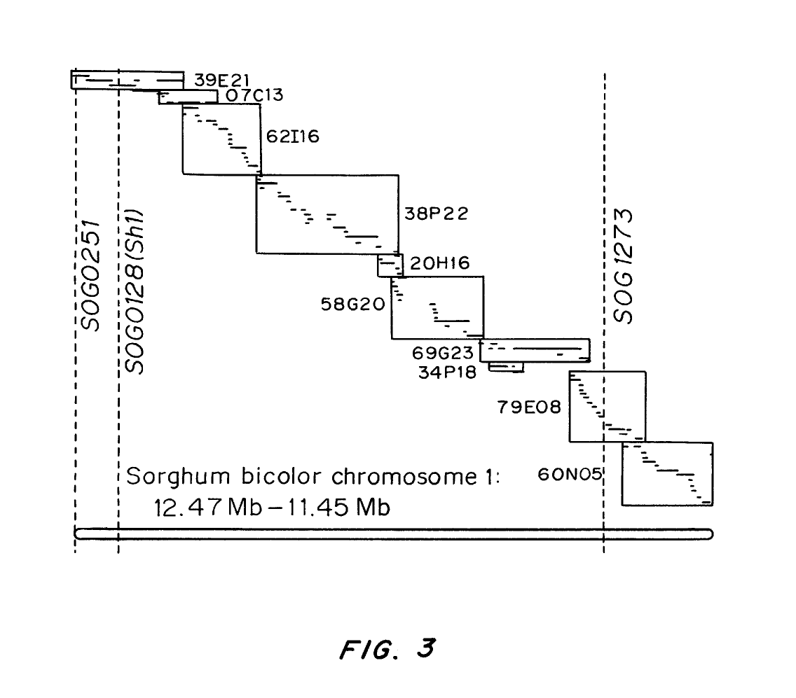

Using the F2 population, the physical location of Sh1 was mapped within a region flanked by two RFLP markers SOG0251 and SOG1273 (FIG. 3), with a genetic distance of 0.42cM (3 recombinants out of a total of 740 gametes) between the two markers. The RFLP markers delineated a genomic region used to identify 10 overlapping S. propinquum BACs in a minimum tiling path (FIG. 3). The sequence reads from the BACs were pooled and assembled into 30 contigs, comprising a total size of 1.04 Mb (N50=63.9 Kb) of sequences from the target region in S. propinquum.

The corresponding regions in S. bicolor and S. propinquum were aligned using MUMMER version 3.0 (Kurtz, et al., Genome Biol, 5:R12 (2004)). The alignments show that the BAC sequences correspond to a ˜1 Mb region on S. bicolor chromosome 1 (FIG. 3). Over 90% of this sequence is well aligned with S. propinquum contigs.

Genome alignments between S. propinquum BACs with the corresponding region in S. bicolor identified 127 sequences (>300 bp) present in S. bicolor but not in S. propinquum. Comparative analyses between S. bicolor and S. propinquum coding regions show that they are very similar at the DNA level. The gene predictions revealed 95 S. propinquum gene models with a median size of 906 base pairs on the sequenced BACs. Among the 95 gene loci predicted, 9 loci show no protein sequence change between S. bicolor and S. proqinquum. The median of synonymous substitution per synonymous site (Ks) is 0.0215 in the shattering region. This median Ks value corresponds to ˜1.7 million years of divergence between S. propinquum and S. bicolor, using a rate estimate of 6.5×10⁻⁹ synonymous substitutions per year (Gaut, et al., Proc Natl Acad Sci USA, 93:10274-79 (1996)). A total of 80 genes are within the boundary of the two flanking markers in the linkage mapping.

Some of the sequences missing in S. propinquum are simple sequence repeats (SSRs) and known retrotransposons. This resource of genomic indels is useful for the discovery of novel transposon species. Because most sorghum helitrons lack structural features compared to other DNA transposons, helitron prediction software can use the indel differences between closely related species as a training set (Du, et al., BMC Genomics, 9:51 (2008)). These indel sequences that are different between the two species of Sorghum were used to train the helitron prediction software used in describing the sorghum genome sequence (Paterson, et al., Nature, 457:551-56 (2009)).

The physical to genetic distance ratio was calculated, which appeared non-uniform in this region. From marker SOG0251 to SOG0128 (˜70 kb, 2 recombinants), where most of BAC YRL39E21 sits, the physical to genetic distance ratio is ˜260 kb/cM (kilobase/centimorgan), whereas between SOG0128 to SOG1273 (˜790 kb, 1 recombinant), the rest of the BACs, the physical to genetic distance ratio is ˜5600 kb/cM, indicating that recombination is very limited in this part of the region. According to previous estimates, heterochromatic regions in sorghum showed a much lower recombination rate ˜8700 kb/cM compared to euchromatic regions ˜250 kb/cM (Kim, et al., Genetics, 171:1963-76 (2005)). Therefore the drastic transition observed in the Sh1 region from one side of the middle SOG0128 marker to the other side is comparable to the difference between euchromatin to heterochromatin, although the region generally appears to be euchromatic (Bowers, et al., Proc Natl Acad Sci USA, 102:13206-11 (2005)). Such a precipitous transition is unlikely an artifact due to sampling: assuming that the low-recombination part has an actual physical to genetic distance ratio of 260 kb/cM, 22 recombinant gametes were expected instead of only 1 observed (P=6×10⁻⁹).

It is unclear what has caused the difference in recombination frequency in this region. The two parts appear to have similar repeat and gene density (FIG. 2). One possibility is that there might be chromosomal inversion to suppress recombination between S. bicolor and S. propinquum in the right part of the region. However, due to the incompleteness of the S. propinquum assembly, this possibility was not tested.

### Example 3

### The Shattering Region Aligns to Homologous Regions in Other Taxa

Gene content and collinearity is conserved across the sorghum shattering region, aligning well with a region on rice chromosome 3 (26.91 Mb-25.79 Mb, i.e. in reverse orientation). Although the rice genome is smaller than sorghum (430 Mb versus 730 Mb), the corresponding region in rice appears to cover a slightly larger physical distance than the sorghum region, although with a similar number of genes (98 versus 95). A total of 77 sorghum genes in the shattering region have syntenic rice orthologs with a median Ks value of 0.58, corresponding to ˜44.6 million years of divergence.

Because of the most recent cereal polyploidy event, the shattering region is also syntenic to rice chromosome 12 (27.23 Mb-26.54 Mb), as part of a duplication block ρ6 (Paterson, et al., Proc Natl Acad Sci USA, 101:9903-08 (2004)). The region is also involved in a more ancient duplication block σ8 (consisting of ρ4 and ρ6) (Tang, et al., Proc Natl Acad Sci USA, 107(1):472-77 (2009)).

Corresponding regions in a eudicot genome are less clear. Part of the sorghum shattering region is syntenic to regions on grape chromosome 6 and chromosome 8 through ancestral synteny block PAR21 (Tang, et al., Proc Natl Acad Sci USA, 107(1):472-77 (2009)), but these synteny relationships are more degenerate, involving less than 10 gene pairs each.

### Example 4

### Shattering Phenotypes are Present in a Sorghum Diversity Panel

### Materials and Methods

Compiling a Sorghum Diversity Panel for Mapping the Shattering Trait

To test the gene-trait association and identify functional candidates in the region, a diversity panel of sorghum varieties that are suitable to study the shattering trait was compiled. These sorghum accessions were provided by S. Kresovich and M. Hamblin from Cornell University and from the USDA-ARS germplasm collection. Within the panel, the varieties were selected to represent a wide range of geographical locations including Africa and Asia (Table 2). Diverse varieties from wider geographical areas are chosen since in theory association mapping works better on unrelated individuals. Otherwise, if some individuals with similar genotypes are represented multiple times in our panel, this could create false positive associations.

There were three accessions that did not flower. In the “PGML index” column accessions with prefix (AL, AN, AP) are from Cornell and accessions with prefix BP are from USDA-ARS. “Race” information was taken from the accompanying documentations shipped with the samples.

TABLE 2
The sorghum accessions selected in the shattering diversity panel.

| | | | |
|---|---|---|---|
| Accession ID | PGML index | Race | Origin |

Complete shatterers (11 varieties)

| | | | |
|---|---|---|---|
| PI 267436 | BP03 (#5) | bicolor | India |
| PI 569834 | BP10 (#6) | bicolor | Sudan |
| PI 521356 | BP06 (#7) | drummondii | Kenya |
| PI 365024 | BP05 (#8) | verticilliflorum | South Africa |
| L-WA 27 | AL03 (#10) | verticilliflorum | Angola |
| L-WA 23 | AL02 (#11) | verticilliflorum | Angola |
| L-WA 13 | AL01 (#12) | verticilliflorum | Sudan |
| PI 155675 | BP01 (#15) | bicolor | Malawi |
| S. propinquum | SP (#20) | S. propinquum | — |
| KFS (deciduous mutant) | KFS (#21) | bicolor | United States |
| PI 570917 | BP11 (#22) | bicolor | Sudan |

Non-shatterers (13 varieties)

| | | | |
|---|---|---|---|
| PI 221607 | AP02 (#1) | bicolor | Nigeria |
| PI 302115 | BP04 (#2) | verticilliflorum | Australia |
| PI 152702 | AP01 (#3) | bicolor | Sudan |
| NSL 87902 | AN07 (#4) | bicolor | Cameroon |
| NSL77217 | AN05 (#9) | bicolor | Chad |
| NSL56003 | AN03 (#13) | bicolor | Kenya |
| NSL56174 | AN04 (#14) | bicolor | Ethiopia |
| PI 267408 | AP03 (#16) | bicolor | Uganda |
| PI 563146 | BP07 (#17) | bicolor | Sudan |
| PI 267539 | AP04 (#18) | bicolor | India |
| PI 563474 | BP09 (#19) | bicolor | United States |
| PI 591385 | BP13 (#23) | bicolor | India |
| PI 584089 | BP12 (#24) | bicolor | Uganda |

### Results

The shattering phenotype for each accession in the panel was carefully validated. A simple but subjective method is to classify the shattering phenotypes of the individuals into “shattering” and “non-shattering”, through the hand tapping technique. The panicles were cut off from the plant and shaken vigorously, and the grains from the “shattering” varieties would usually fall off easily. Alternatively, breaking tensile strength (BTS) was used as a quantitative measurement for the degree of shattering (Konishi, et al., Science, 312:1392-96 (2006)), using a digital force gauge (IMADA Inc. DPS-4) to clasp to the grain and measure the force required to break the pedicel when pulling the grain away. The BTS values were recorded at different developmental stages and stable values (after maturity of the grains) were used to distinguish the shattering/non-shattering phenotype for each variety. For each genotype, the BTS values was recorded for multiple panicles at roughly five-day intervals. Ideally, the sorghum accessions need to be measured at roughly equally spaced dates. However, since different sorghum accessions were flowering at different times, it is difficult to track each individual panicle and manage a well spaced sampling of measurements. Therefore, a few accessions were not sampled every five days.

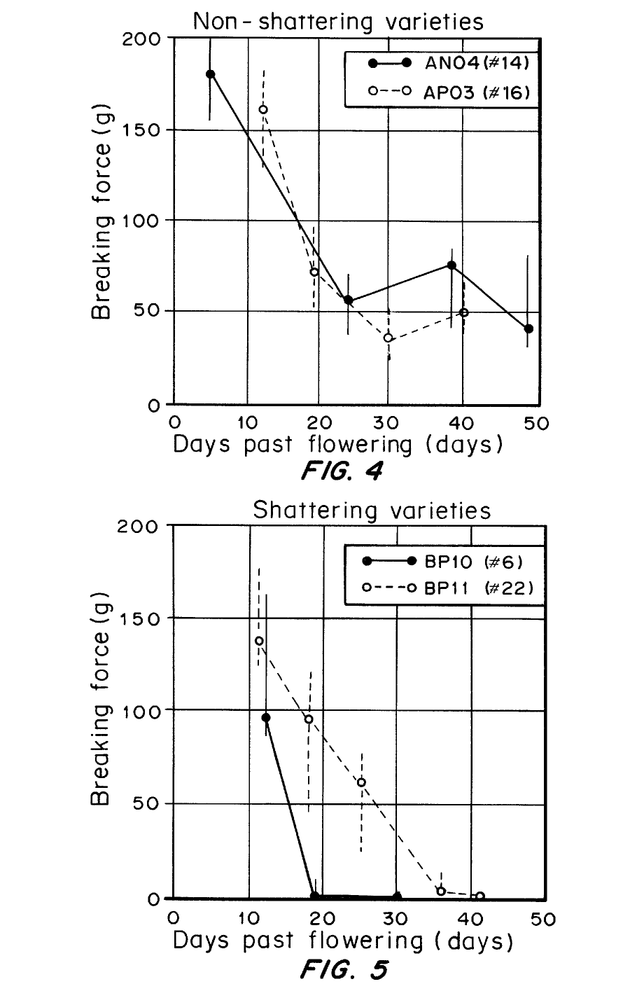

In the span of five months, a total of 77 panicles were clipped from the planted sorghum individuals and measured in terms of degree of shattering at various stages (multiple panicles were measured for each genotype). On average, each panicle was tracked and measured around 4 times, with one case (AP03, panicle #8) measured 8 times to make sure that it is indeed non-shattering. The shattering varieties are often easier to distinguish since they are deciduous once the grains mature, while the non-shattering varieties need to be monitored for a longer period of time. It was found that the breaking force (BTS) for non-shattering varieties stabilize around 50 g force after maturity, while the shattering varieties go to zero, i.e. capable of dispersal with little external force (FIGS. 4 and 5).

The final distributions of the mature BTS for the genotypes are therefore quite bimodal even without the quantitative measurements. 25 g of mature BTS was used as a cutoff to distinguish the shattering/non-shattering genotypes, and 23 panicles (from 8 varieties) were scored as shattering and 52 panicles (from 13 varieties) were scored as non-shattering. These results are consistent with the qualitative hand tapping. One individual (BP06) did not flower in the five month period, so the plant was moved to the growth chamber to induce flowering. BP06, KFS and SP were not measured with force gauge but were verified as “shattering” varieties through hand tapping. The final phenotypes for the sorghum individuals are shown in Table 2.

### Example 5

### Linkage Disequilibrium in the Sh1 Region

### Materials and Methods

Resequencing and Analyses of the Polymorphic Sites within the Shattering Region

Primers of 20-22 bp that amplify between 700-1000 bp amplicons were designed around the polymorphic sites of the candidate loci using PRIMER3 (Koressaar, et al., Bioinformatics, 23:1289-91 (2007)). DNA was prepared from young leaves of individual plants. PCR reactions of 15 μl per well were set up to amplify sampled regions using the following thermo-cycling program (ANN): 95° C. 30 sec, 58° C. 30 sec, 72° C. 1 min for a total of 36 cycles, 72° C. 10 min. The concentrations of the PCR amplicons were verified in 1% agarose gel and excessive primers and dNTPs in the PCR reactions were removed using exonuclease I and shrimp alkaline phosphatase enzymatic digestion. The amplicons were sequenced using BigDye 3.1 chemistry using the following thermo-cycling program (BRISEQ): 96° C. 15 sec, 56° C. 30 sec, and 58.8° C. 1 min 30 sec for a total of 60 cycles. Excessive primers and dyes in the sequencing reactions were removed using Sephadex columns before the sequencing plates were loaded onto ABI3730 capillary sequencer.

The chromatograms were examined carefully using SEQUENCHER software (GENECODES Inc. version 4.1) and the polymorphisms were recorded in an EXCEL spreadsheet. From each PCR amplicon sequence, only the “informative” SNPs (tagging SNPs that are sufficient to reconstruct haplotype blocks) were retained based on the observation that polymorphic sites within the same amplicon often show complete linkage disequilibrium (LD). PCR amplicons were sequenced with the DNA of 24 individuals in the compiled shattering panel. The public genome sequence of sorghum was from a non-shattering inbred cultivar S. bicolor BTX623 (Paterson, et al., Nature, 457:551-56 (2009)), therefore a total of 25 different genotypes were available to be compared.

LD between multiple loci and the strength of marker-trait associations were analyzed using TASSEL (version 2.1) (Bradbury, et al., Bioinformatics, 1;23(19):2633-5 (2007)). r² was used as an indicator of linkage disequilibrium between pairwise SNP markers. Consider a pair of loci—alleles A/a in one and B/a in another, π_{A}, πₐ, π_{B}, π_{b} are allele frequencies, π_{AB}, π_{aB}, π_{Ab}, π_{ab} are haplotype frequencies, then the following equation can be used (Flint-Garcia, et al., Annu Rev Plant Bio, 54: 357-74 (2003)),

$${r}^{2} = \frac{{( {π}_{AB} - {π}_{A} {π}_{B} )}^{2}}{{π}_{A} {π}_{a} {π}_{B} {π}_{b}} .$$

For the association test, a generalized linear model (GLM) was used to evaluate the level of association between the shattering traits with the genotype data. Sorghum propinquum genotype was excluded from the calculations of LD.

### Results

A total of 67 informative sites were retained after removing a few sites with rare polymorphisms. The concatenated 67 sites comprise haplotype alignment among the individuals and were used as input to the program TASSEL. Some sites are heterozygous for some individuals (e.g. plant #24 is heterozygous in least three sites). A total of 5 sites are indels (ranging from 3 to 11 bp), but are treated similarly as SNP sites in the analysis.

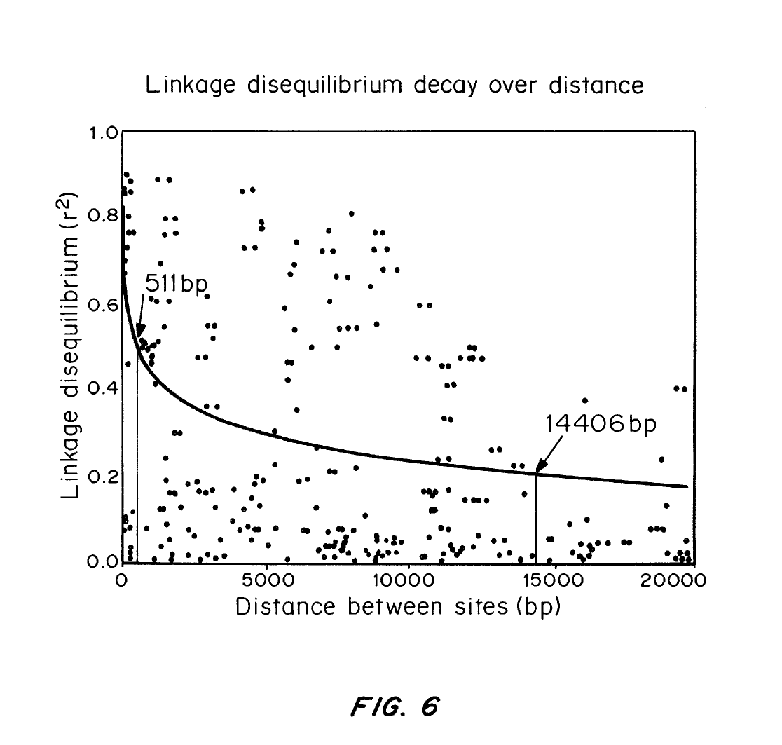

Compared to maize, sorghum is a predominantly self-pollinating species with a range of outcrossing rates between 2%-35%; Sorghum also has a smaller effective population size. Both factors can lead to higher levels of LD than maize (Hamblin, et al., Genetics, 167:471-83 (2004)). The strength of LD over the physical distance is shown in FIG. 6. The LD in this region drops by half at a distance of ˜500 bp. This estimate of LD is largely consistent with a previous estimate of LD decay to 0.5 by 400 bp (Hamblin, et al., Genetics, 167: 471-83 (2004)).

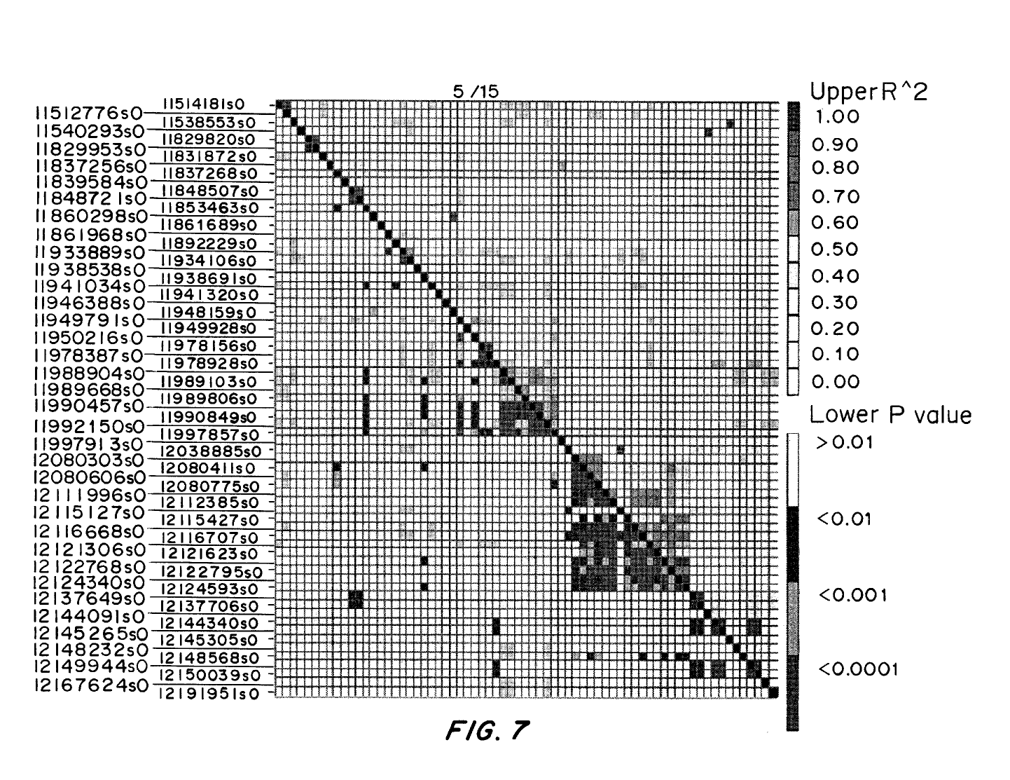

Pairwise LD values between the sampled sites were shown in FIG. 7. Two relatively large LD blocks (with size ˜48 kb and ˜44 kb) were evident. Although the average estimate for LD decay as calculated above was 477 bp, in the two large LD blocks in FIG. 7, sites that were separated by 40 kb still showed LD ˜0.5. There was also variation of LD in the region, as some regions do not show strong LD. This might have been partially affected by the uneven sampling of polymorphic sites. Some LD occasionally persisted over large distances and did not correspond to the tight linkage, as suggested in (Flint-Garcia, et al., Annu Rev Plant Biol, 54:357-74 (2003)).

### Example 6

### Association Analysis in the Sh1 Region

The general linear model (GLM) used is a simple statistical model: y=marker+e, where y is the phenotype (0 for non-shattering, 1 for shattering). Since only a specific target region was searched, the risk of false positive associations is much less than for a genome-wide search, mitigating the need for inclusion of population structure parameters in the model.

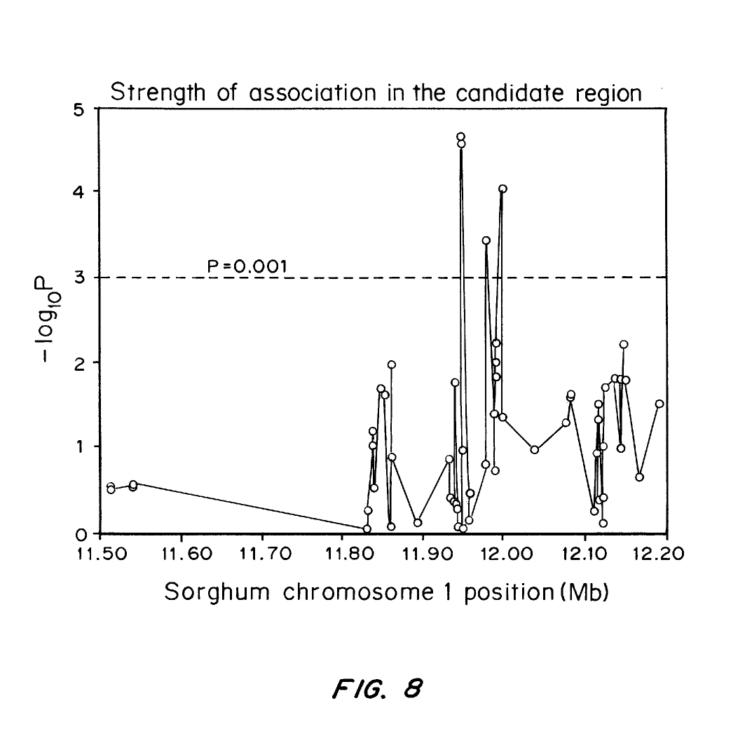

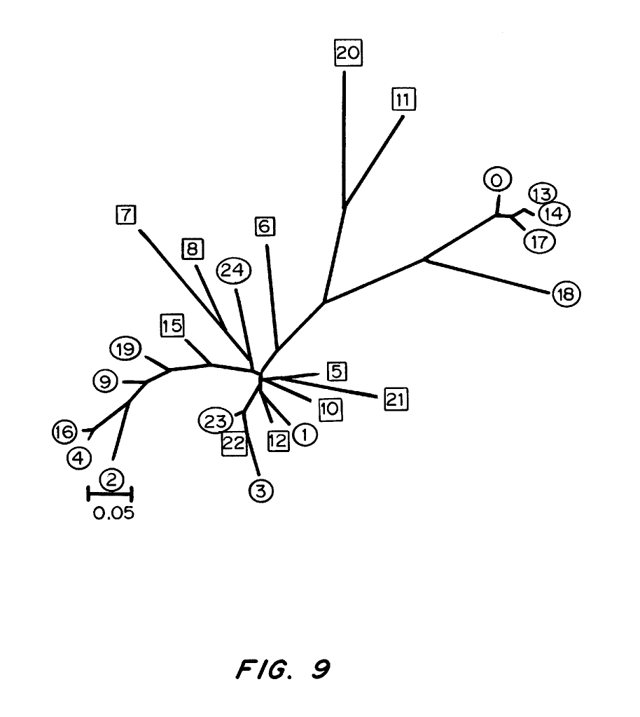

Among the 67 sites that were tested, 4 sites were found significantly associated with the shattering trait (amplicons P7E9, P3H11, P8F9 and P4C3 in the shattering region) at significance level P<0.001 (FIG. 8; FIG. 9). The highest peak contains P7E9 (P=2.8e-5) and P3H11 (P=2.2e-5), covering a ˜50 Kb genomic region. The four sites were also in good LD. However, the intermediate sites between the two peaks were not significantly associated with the shattering trait, possibly due to mutations that are of more recent origin than those related to shattering and therefore are not informative with regard to shattering.

TABLE 3
Four sites with strong associations with the shattering trait (N/S).

| Phenotype | | N | N | N | N | N | N | N | N | N | N | N | N | N | N |
|---|---|---|---|---|---|---|---|---|---|---|---|---|---|---|---|
| Coord | Marker | 0 | 2 | 4 | 9 | 13 | 14 | 16 | 17 | 19 | 23 | 3 | 1 | 18 | 24 |
| 11949791 | P7E9 | A | A | A | A | A | A | A | A | A | A | ? | B | C | B |
| 11950216 | P3H11 | A | A | A | A | A | A | A | A | A | A | A | B | B | B |
| 11978928 | P8F9 | A | A | A | A | A | A | A | A | A | A | A | A | A | ? |
| 11997857 | P4C3 | A | A | A | A | A | A | A | A | A | B | B | B | B | B |

| Phenotype | | S | S | S | S | S | S | S | S | S | S | S |
|---|---|---|---|---|---|---|---|---|---|---|---|---|
| Coord | Marker | 5 | 6 | 7 | 8 | 10 | 11 | 15 | 20 | 21 | 12 | 22 |
| 11949791 | P7E9 | B | ? | B | B | B | B | B | B | B | B | A |
| 11950216 | P3H11 | B | B | B | B | B | B | B | B | B | B | A |
| 11978928 | P8F9 | A | ? | B | B | B | B | B | B | A | A | A |
| 11997857 | P4C3 | B | B | B | B | B | B | B | B | B | B | B |

Each column represents the genotype from one individual.
Symbol “A” represents S. bicolor BTX623 type (individual #0);
Symbol “B” represents different allele;
Symbol “C” represents heterozygous;
Symbol “?” represents missing data.

Additional PCR primers were designed to sample more sequences in the ˜50 kb region which extends from gene models Sb01g012870 to Sb01g012960, in order to find the extent of the LD and also reveal sites that are even more associated with the shattering trait that might be the actual causal site or tightly linked sites. If the causal locus Sh1 is assumed to have perfect association with the shattering trait, the r₂ between P3H11 and Sh1 is 0.48—a relatively tight linkage based on the LD decay trend in FIG. 6. Based on the genotypes within this region, it is likely the Sh1 locus is further contained between base position 11,946,388 to 11,956,003. This interval contains two genes, encoding two transcriptional factors Sb01g012870 and Sb01g012880, both of which are located within BAC YRL20H16 (FIG. 10A).

### Example 7

### Relationship Among the Genotyped Individuals

Phylogenetic relationship was also observed among the haplotypes of the individuals. Visually, three sub-structures were seen, note that #0 and #20 are the two parents used in the linkage mapping study (FIG. 9). One clade contained S. bicolor BTX623 (#0) with four other non-shattering varieties, one clade contained S. propinquum (#20) and one other shattering variety, while the rest formed the third clade with mixed shattering/non-shattering accessions.

The tree analysis was used to determine whether there is underlying population structure that accounts for the shattering/non-shattering varieties. If this were the case, then the associations identified above might be false positives. This is unlikely, for two reasons. First, clade #3 in FIG. 9 includes both shattering/non-shattering individuals and therefore does not show significant partitions. Second, most sites in the region do not show significant association with the trait (except for the three sites shown in FIG. 9).

### Example 8

### Sb01g012870 and Sb01g012880 are Candidates for the Sh1 Gene

A candidate genomic region that contains all four associated sites (FIG. 8) extends from gene model Sb01g012870 to Sb01g012960, which covers ˜50 kb of sequence and ˜10 predicted genes. Based on the genotypes within this region, the Sh1 locus can be contained between base positions 11941320 to 11956003, also supported by two SNP sites with highest significance (FIG. 8, and FIG. 10A). This interval only contains two genes, encoding two transcriptional factors Sb01g012870 and Sb01g012880.

Sb01g012870 is a member of the WRKY gene family, and is implicated in a variety of physiological and developmental processes including leaf senescence in Arabidopsis (Robatzek, et al., Plant J, 28:123-33 (2001)). Interestingly, over-expression of this gene could result in ectopic lignin deposition, as reported in Medicago (Naoumkina, et al., BMC Plant Biol, 8:132 (2008)), tobacco (Guillaumie, et al., Plant Mol. Biol., 72(1-2):215-34, (2009)) and rice (Wang, et al., Plant Mol Biol, 65:799-815 (2007)).

To verify the predicted gene models, the full length cDNAs from both shattering S. propinquum (Sh1) and non-shattering S. bicolor (sh1) were sequenced. The transcript from the Sh1 allele encodes a 144-amino-acid protein. The transcript from the sh1 allele encodes a 100 aa protein. Both proteins contain a 54 aa WRKY domain that show no amino acid differences between the two species. The conserved [WKKYGQK] sequence is considered to be directly involved in DNA binding with downstream DNA motif called W-box (EULGEM™ et al. 2000).

The S. propinquum allele and S. bicolor allele differ at two amino acid positions within this protein (FIG. 10B). Both of the two substitutions are located outside the WRKY domain. Notably, one amino acid difference is at the translational start of the S. bicolor allele, which makes the S. bicolor protein 44 residues shorter than the predicted S. propinquum protein (FIG. 10B). Differences in gene prediction method could have caused this size difference—it is possible that the S. bicolor gene also starts earlier than the model in Paterson, et al., Nature, 457:551-556 (2009) (i.e. at the S. propinquum start site). EST evidences appear to favor the S. bicolor gene model. However, the Sh1 protein cannot start at the S. bicolor start, because of ATG to ATT mutation in Sh1 transcript in this particular codon, which also results in a methionine (M) to isoleucine (I) substitution in the protein sequence (column 61 in FIG. 11A). Data also shows that the S. propinquum transcript appears to be longer than the S. bicolor transcript. The second amino acid difference is a substitution of histidine (H) to glutamine (Q) (column 136 in FIG. 11A).

The next gene, Sb01g012880, is a member of the TATA-box gene family, and is also a transcriptional regulator that is evolutionary conserved across fungi, animals and plants. The two maize orthologs tbp1/2 were studied in (Swigonova, et al., Genome Res, 14:1916-23 (2004)). However, the polymorphic sites between the two sorghum species are all synonymous sites (i.e. they do not show amino acid differences).

Both genes Sb01g012870 and Sb01g012880 are on BAC YRL20H16 contig 13. Both genes can be cloned from the BAC YRL20H16, these two gene fragments enzyme-cut, and the fragments ligated to the transformation vector. In order to make sure that the entire transcriptional machinery of these genes are carried in the vector, additional flanking sequences from both 5′ and 3′ end can also included and cloned.

Because of the dominant nature of the S. propinquum allele, the non-shattering S. bicolor individuals can be transformed. Shattering phenotype can be found in the transformant, as functional validations of these gene candidates.

### Example 9

### Sorghum Sh1 has Homologs in Other Grasses

The WRKY gene family is a large family in plants (e.g. 113 members in rice (Gao, et al., Bioinformatics, 22:1286-1287 (2006)), however, the direct ortholog(s) of Sh1 in the related grass genomes were identified based on genomic collinearity. The comparison of sorghum Sh1 proteins to other sequenced grass genomes showed that Sh1 is orthologous to two maize proteins encoded by GRMZM2G149219 and GRMZM2G161411, two Setaria proteins Si038955m and Si038001m, rice OsWRKY60 (Os03g0657400) and Brachypodium protein Bradi1g13210 (FIGS. 11A and 11B). All of these proteins are each located in the collinear region in the respective genome when compared to the target region on sorghum chromosome 1. It is more difficult to discern the direct orthologs(s) among the 21 similar proteins in grape and 19 proteins in Arabidopsis because of the lack of collinearity between Sh1 and those proteins. The two gene copies in maize were derived from the WGD event (Schnable, et al., Science, 326:1112-1115 (2009)). The two copies in Setaria are tandem gene copies that are adjacent to one another. In both cases, the two duplicated gene loci were able to retain the genomic collinearity to the Sh1 locus due to their non-dispersed duplication mechanism.

We found that the distinction of the long (˜140 aa) and short proteins (˜100 aa) in sorghum also exist in other grass genomes, with the short proteins often lacking a ˜40 aa N-terminus, although the exact N-terminus sequences vary among the long proteins. Based on the exon-intron structures of these homologous genes, the sequences in the 3′-terminal exon are much conserved across the homologs compared to the 5′-end. The main difference among the gene homologs is whether they have 1 or 2 additional exons in the 5′-end, which amounts to either 2 or 3 exons in total (FIG. 11B). The long proteins often contain 3 exons, with the only exception of Os03g0657400 which might have merged the first two exons. On the basis of the codon alignments (not shown), the ATG to ATT mutation (M=>I) appears to be derived in S. propinquum, since all other orthologous genes in the related grass species has a “G” in that nucleotide position. The maize ortholog GRMZM2G161411 has a “TTG” codon which translates to valine (V).

In the grasses compared in this analysis, there is at least one copy of the long protein, while species with two gene copies (maize and Setaria) contain one extra short protein. The rice and Brachypodium ortholog is long, which is the only gene copy in their genomes. There are two copies in maize and Setaria, one short and one long copy. The duplication into two copies in maize and Setaria occurred more recently and independently in their respective lineages after the divergence with other grasses (FIG. 11B).

The extended part in the 5′-end of the Sh1 protein are much less conserved in the grasses compared to the WRKY domain based on the multiple sequence alignments (FIG. 11A). A BLASTP search to Genbank using only the 44 N-terminal amino acids did not reveal any significant hits at E<0.01.

### Example 10

### A Sb01g012870 Transgene Increases Shattering in a Non-Shattering Sorghum Background

### Materials and Methods

RT-PCR of the Gene Candidate

The gene expression profiles were studied through inflorescence development in the shattering and non-shattering genotypes. Plant materials for the phenotyping and expression studies were collected from the University of Georgia Plant Science Farm during a summer season. Sorghum halepense genotype GRIF14527 was chosen to represent the shattering category and S. bicolor genotype PI 658864, a recombinant inbred line derived from a cross between BT×623 and IS3620C, was selected as a non-shattering type. Inflorescence was collected at different developmental stages by visual observation, i.e. inflorescence still covered by flag leaf, inflorescence just emerging from flag leaf, after anther dehiscence and inflorescence close to maturity. Tissue was harvested from two different individuals for each developmental stage. Also leaf samples were collected from each genotype to use as a control. Part of the tissue harvested was flash frozen in liquid nitrogen and stored at −80° C. until RNA isolation. The remainder of the inflorescence was used to score the phenotype.

RNA from inflorescence and leaf tissue was isolated using RNeasy plant mini kit (QIAGEN Inc., Valencia, Calif., USA) according to the manufacturer's protocol. RNA was treated with RNase-Free DNase set (QIAGEN Inc., Valencia, Calif., USA) to digest any genomic DNA which might be present. RNA was quantified using a UV-spectrophotometer. RNA quality and integrity was examined on a 1% agarose gel prepared in RNase free 1X TAE. First-strand cDNA was synthesized from 1 μg of total RNA using SuperScript III reverse transcriptase (Invitrogen) with 500 ng anchored oligo (dT) primers in a 20 μl reaction. This reaction was incubated at room temperature for 5 min prior to 2 hour cDNA synthesis at 50° C. and 15 min at 70° C. After cDNA synthesis 20 μl sterile double-distilled water was added to the reaction. Each PCR reaction consisted of 1 μl cDNA in a 20 μl reaction with the following components: 4 μl 5×GoTaq green reaction buffer, 2 μl 2 mM dNTP mix, 0.5 μl each primer (10 μM), 0.5 Units of GoTaq DNA polymerase (Promega Corporation, Madison, Wis.). The thermal profile consisted of incubation at 95° C. for 4 mins, followed by 35 cycles at 95° C. for 45 sec, annealing temperature for 45 sec, 72° C. for 45 sec, and a final extension at 72° C. for 5 mins. A Sorghum actin gene (SbActin) was used as loading control. The forward and reverse primer sequence for SbActin is as follows: forward 5′-acattgccctggactacgac-3′ and reverse 5′-aatgaaggatggctggaaga-3′.

### Results

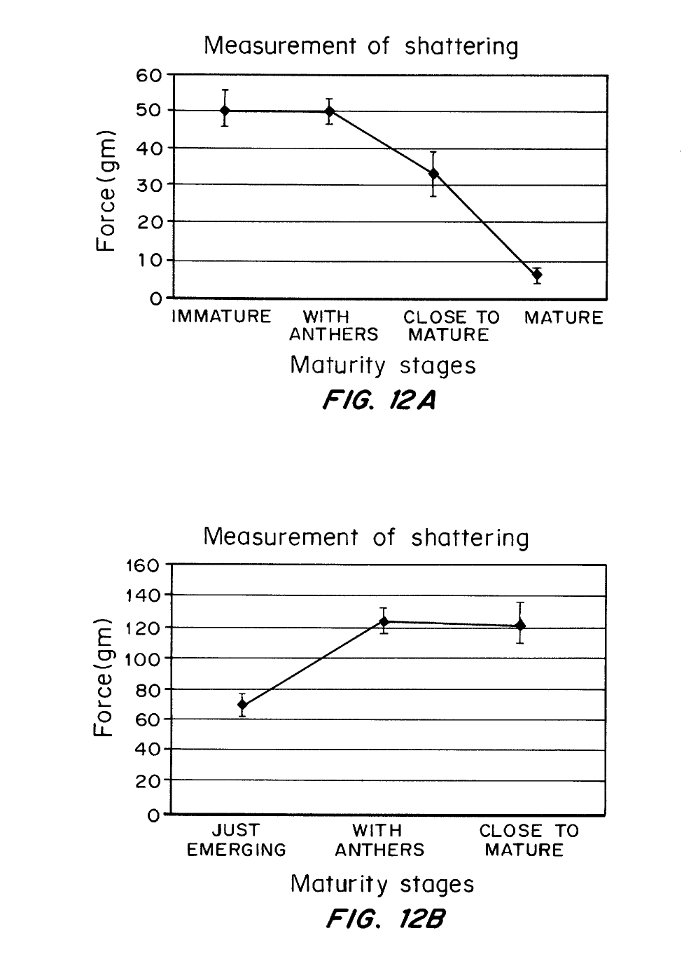

Shattering and non-shattering phenotypes for the two genotypes used for the expression study was confirmed using the breaking tensile strength (BTS) method (discussed above). The BTS values were measured at different floral developmental stages. For each stage ten individual florets were tested from two different panicles. The results are presented in FIGS. 12A and 12B. The BTS value went down rapidly in shattering S. halepense (a tetraploid formed from the cross between S. bicolor and S. propinquum) starting from 55.1 g in immature (just emerged from flag leaf) to 7.5 g in mature inflorescence. In non-shattering S. bicolor the BTS value actually increased in the inflorescence after anther dehiscence compared to immature inflorescence (123.1 g and 69.8 g respectively) and it remained consistent even in the mature inflorescence (122 g) without any significant drop in breaking tensile force.

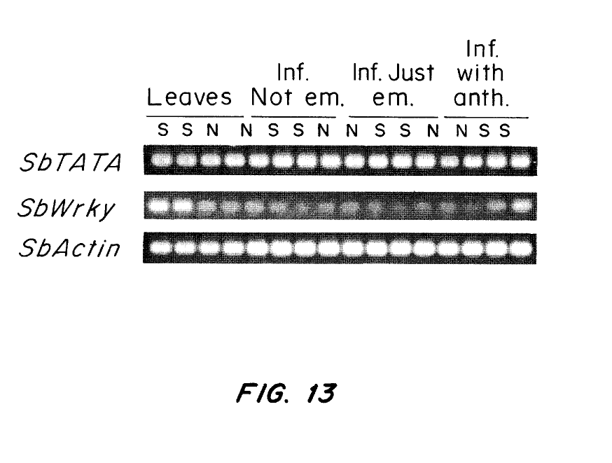

Semi-quantitative RT-PCR was run to investigate the expression profile of the Sh1 gene. A sorghum actin gene was used as a loading control. Primers for both Sh1 were designed from the CDS of the respective genes and two primer pairs were tested yielding similar results. Data from one of the primer pairs are shown in FIG. 13. Sh1 was expressed strongly in leaves in shattering S. halepense but the expression level went down in inflorescence gradually towards more mature developmental stages. Sh1 was also expressed in leaves of non-shattering sorghum but in inflorescence it had weaker expression until the anther dehiscence stage where the expression of this gene was very strong when compared to other stages. This indicates that this gene might be playing an active role in shattering and the particular developmental stage is critical for manifestation of the trait.

in some grasses, shattering is a quantitative trait (rice and maize each have multiple genes, for example) but in sorghum it is discrete (Paterson, et al., Loci. Science, 269:1714-1718 (1995a)). The QTLs affecting shattering on maize chromosomes 1 and 5 (Paterson, et al., Loci. Science, 269:1714-1718 (1.995a)) harbor GRMZM2 G149219 and GRMZM2G161411 respectively. GRMZM2G149219 is a “short” protein with 99 amino acids, while GRMZM161411 is a “long” protein with 140 amino acid residues. Since both maize genes fail in the identified shattering QTL intervals, both the long copy and the short copy might be involved in the shattering pathway in maize.

Sh1 contains the WRKY DNA-binding domain, and belongs to a superfamily of plant transcriptional factors. Members of this family have been implicated in a variety of physiological and developmental processes that are unique to plants, including leaf senescence (Robatzek, et al., Plant J, 28:123-133 (2001) and Robatzek, et al., Genes Dev, 16:1139-1149 (2002)), trichome initiation (Johnson, et al., Plant Cell, 14:1359-1375 (2002)) and embryo morphogenesis (Lagace, et al., Planta, 219:185-189 (2004)). The WRKY domain functions through the direct interactions with the W-box domain in the promoter region in the downstream gene targets (Eulgem, et al., Trends Plant Sci, 5:199-206 (2000)). Over-expression of gene homologues in different plant systems were shown to result in ectopic lignin deposition, as reported in Medicago (Naoumkina, et al., BMC Plant Biol, 8:312 (2008) and Wang, et al., Proc Natl Acad Sci USA, 107:22338-22343 (2010)), tobacco (Guillaumie, et al., Plant Mol Biol, (2009)) and rice (Wang, et al., Plant Mol Biol, 65:799-815 (2007)). In particular, Wang and co-workers isolated a WRKY gene in Medicago and Arabidopsis, when disrupted, showed secondary cell wall thickening associated with the deposition of lignin, xylan and cellulose (Wang, et al., Proc Natl Acad Sci USA, 107:22338-22343 (2010)).

The expression of Sh1 is up-regulated during the anther dehiscence stage of floral development of the shattering sorghum suggests that Sh1 might be a positive regulator. The downstream targets of Sh1 is not yet known but other members in the WRKY family is known to regulate cell wall biosynthesis genes (Wang, et al., Proc Natl Acad Sci USA, 107:22338-22343 (2010)).

Towards the end of the floral development in the beginning of the shattering process, there is significant lignin deposition at the seed-stalk interface. The lignification of those tissues is part of the programmed cell death and facilitates the break-off of the seeds from the stalk. The lignin stain (phloroglucinol) of seed pedicel from the non-shattering sorghum revealed no deposition of lignin and consequently less ease in breaking off this tissue interface. Fluorescent microscopic analysis of the seed-stalk showed that the reddish stalk part has entirely no fluorescence compared to the relatively high fluorescence seen in the seed skin, which suggests that there is no lignin deposition near the shattering zone.

### Transformation of a Candidate Gene into Non-Shattering Sorghum Increases Shattering

The candidate genes that are in the high association region (Sb01g012870, Sb01g012880) (FIG. 10A) from the BAC YRL20H16 were cloned by cutting the gene fragments using restriction enzymes, followed by ligation of these fragments onto the transformation vector. The background was T×430, which is a non-shattering sorghum cultivar. To make sure that the entire transcriptional machinery of these genes are carried in the vector, additional flanking sequences that contain likely cis-regulatory elements from both 5′- and 3′-end were also included and cloned along with the coding sequences.

We confirmed the presence of the shattering allele in transformants using two pairs of primers. The primers span the first intron in S. propinquum which is longer than the corresponding sequence in S. bicolor. Stringent annealing temperature and 40 PCR cycles were used. The band patterns show two bands of distinct sizes—smaller band in S. bicolor, larger band in S. propinquum and both bands in transgenics. Among the transgenic tested, only T3 shows a single S. bicolor-sized band therefore seems to be not transformed.

The transgenic sorghum were grown out to test if the construct can induce shattering. The Sb01g012870 construct (SEQ ID NO:4) induced seed dropping in a few sorghum transformants. When mature heads were hit the seeds dropped off rather easily. Other transformation events carrying plasmids with the other gene Sb01g012880 (SbTATA) and controls did not show easy seed dropping.

To further quantify the effect of the Sb01g012870 construct on seed shattering, for nine different transformed plants containing different transformation events, we grew and evaluated up to 2.4 self-pollinated progeny. The transgene was segregating in 8 of the 9 progeny groups (one group lacked the transgene, possibly indicating that it had not been integrated into the nucleus in the original transgenic plant). Across 136 plants from the eight validated events, reduced breaking tensile strength (BTS) was highly correlated with presence of the transgene (r=−0,641, P<<0.01, with correlations in the individual populations (events) ranging from −399 to −0.946. Segregants that lacked the transgene showed average BTS of 57.8 (St. dev=13.99, n=38), indistinguishable from that of the population that lost the transgene (52.4, St. dcv=15.7, n=17). Plants containing the transgene had significantly smaller average shattering force (22.3, St. dev=18.6. n=105).

TABLE 4
Results of breaking tensile strength (BTS) assay

| | BTS | St Dev. | n |
|---|---|---|---|
| T1 segregants lacking the transgene | 57.80 | 13.99 | 29 |
| T1 segregants with the transgene | 22.52 | 19.47 | 104 |
| T1 population ZG220-1-10b, lacking the | 52.43 | 15.74 | 17 |
| transgene | | | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. An isolated nucleic acid comprising a nucleic acid sequence selected from the group consisting of (1) a nucleic acid sequence at least 90% identical to SEQ ID NO:1, 2, 3, 4, 5, 6, or a complement thereof (2) a nucleic acid sequence of a polynucleotide that hybridizes under stringent conditions to a polynucleotide consisting of the nucleic acid sequence SEQ ID NO:1, 2, 3, 4, 5, 6, or complement thereof (3) a nucleic acid sequence encoding SEQ ID NO: 12, 13, 14, or 15, or a complement thereof and (4) a nucleic acid sequence of a polynucleotide that hybridizes under stringent conditions to a polynucleotide encoding SEQ ID NO: 12, 13, 14, or 15, or a complement thereof.

2. A recombinant expression vector comprising the isolated nucleic acid of claim 1 operably linked to an expression control sequence.

3. The recombinant expression vector of claim 2, wherein the expression control sequence is a heterologous expression control sequence.

4. The recombinant expression vector of claim 3, wherein the expression control sequence comprises a constitutive promoter.

5. The recombinant expression vector of claim 3, wherein the expression control sequence comprises a tissue specific promoter.

6. A isolated polypeptide comprising an amino acid sequence of SEQ ID NO:12, 13, 14, or 15, or variant thereof comprising at least 90% sequence identity to SEQ ID NO: 12, 13, 14, or 15.

7. A transgenic plant or transgenic plant cell, comprising an expression control sequence operably linked to a first polynucleotide that silences expression of a second polynucleotide having a nucleic acid sequence at least 90% identical SEQ ID NO:1, 2, 3, 4, 5, 6, 7, 8, 9, 10, 11, or a nucleic acid encoding SEQ ID NO: 12, 13, 14, 15, 16, or 17.

8. The transgenic plant or plant cell of claim 7, wherein transcription of the first polynucleotide in the plant or plant cell reduces expression of a gene endogenous to the plant, wherein the gene is involved in the development of a dehiscence zone and valve margin of a fruit in the plant.

9. The transgenic plant or plant cell of claim 8, wherein the second polynucleotide has a nucleic acid sequence at least 90% identical to SEQ ID NO:1, 2, 3, 4, 5, 6, or a nucleic acid sequence encoding SEQ ID NO: 12, 13, 14, 15, and wherein the transgenic plant has reduced seed shattering compared to a non-transgenic plant of the same species while maintaining an agronomically relevant threshability.

10. The transgenic plant or plant cell of claim 9, wherein the transgenic plant has reduced lignin deposition around the seed-stalk interface compared to a non-transgenic plant of the same species.

11. The transgenic plant or plant cell of claim 10, wherein the species of the transgenic plant is S. propinquum.

12. The transgenic plant or plant cell of claim 8, wherein the second polynucleotide has a nucleic acid sequence at least 90% identical to SEQ ID NO: 7, 8, 9, 10, 11, or a nucleic acid sequence encoding SEQ ID NO: 16 or 17, and wherein the transgenic plant has increased seed shattering compared to a non-transgenic plant of the same species.

13. The transgenic plant or plant cell of claim 12, wherein the transgenic plant has increased lignin deposition around the seed-stalk interface compared to non-transgenic plant of the same species.

14. The transgenic plant of claim 13, wherein the species of the transgenic plant is S. bicolor.


SEQUENCE LISTING (23 sequences)

SEQ ID NO: 1 - DNA, 435 bp, Sorghum propinquum
atggattcaagctcacagcccggcgcaattgatacatgcagagggagcggaggaggaggagatagaaaccaaagggaggaggacgcggcggcggcggcggcggcagaggccggctacggcaggcagctggtgattcccgaggacgggtacgagtggaagaagtacggccagaagttcatcaagaacatccagaaaatcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcagggcggcggcgcctccgacttcaacagatacgagctgggcgcgcagtacttcggcggggccggccggtcgcattga

SEQ ID NO: 2 - DNA, 1464 bp, Sorghum propinquum
atggattcaagctcacagcccggcgcaatgtatgcatctctctctctctctctctctctctctctctctctctctctctctctctctctctctctctctctacatcatcgtttgggggatgaatcaaatggggctgccaattatcaaggaatgaatggtttttgttcaccctccttatattagtctttctctctacgctgtgtttggtgcgtttgccttaaaccacactcggtgtattaggggttggcaacttatcatagctttggttctcatgcatgcatgtatggttcatcatgtttttgtcaaattttcatgtagcaacatattgtcctccgtccacaacagataagctgatcctgctagtcatagctgctatatacagatcagcttattaagtttgcatcattgtagaagcaaaagtaattaagcacccgggcggcagacatgttacgtacgtatataacaggttgttgttatgcgtgttctaatgttccttggcacaacaactgtagtgatacatgcagagggagcggaggaggaggagatagaaaccaaagggaggaggacgcggcggcggcggcggcggcagaggccggctacggcaggcagctggtgattcccgaggacgggtacgagtggaagaagtacggccagaagttcatcaagaacatccagaaaatcaggtacttgctccgttcgatccaacatatgcatacgtagcatttttggcatcgagattgatctcgagctctcaaataaagctagtgcaaacttgatcacatataccattttttcgtggtcaaatctcgtttcccgccatacgcgtgtacatcagattaatcaatagctcgacgttgaccaagcttgttgacttgttcatcttcgttcctgtgcatcaaatcgttttattaattaattgagtcgatgtgacgcccatcgatcgatcactggtataatggaatgtatgggttgcccgccgtccccgtgcatatatgcatacgtgcaatgctctgctgccagatcttatctttcgaagaagaatcaacggaagaataatatcctcgctttattatattatatattgataacggtcgaccaaataaagccctgatgatgacttgatgagcaaactgcacaagtgtgttttgcattgcatgccaactgatgataccaccgtacgtgggtggtccatgatgcatgtgtgtgatcaaaatccaacaatggcgcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcagggcggcggcgcctccgacttcaacagatacgagctgggcgcgcagtacttcggcggggccggccggtcgcattga

SEQ ID NO: 3 - DNA, 3215 bp, Sorghum propinquum
taagatgactctattttttatcaataagcactttgtactatgattaagacaaaaggaagagaggggacaagaattacaaactatacttaggggttgtttgaatttcagtcatagttggtcacaactcagatgtggtgagacacactctatgatgagaataatgagatctgtttggttctcttctcacctaggctacatcgcatctggagcgagagacaggctagccacagcctggtctggtgcatgcacctgcacttgtttggttttgctctttgttttgagccactccagccatgtctcggaaagatattgttttgttggtctttggcttggcaccagtgctctctcacgcgtacaggcacacgctctcttttggctccacgcagccatgtgttggctaaaaatgattttagaatccatttcccatgagcctgagatggttgcacgcactataggtctaaccctggtagcactttaggtaaccaaacaccttaagcctgcatcccaagagccaggccagtttggaaactggacaaccaaataggcctctaatgaatttgatgtgttgtattctgtgggtgtctagcactcttcaccaactaaacactgataaaaaaaagttatggtgtgcgatgccttagtgtggcatagcaagtgaaggccgggaaccaaacatgcttttactctttcatatcttaggccatgtttggtttgtcgtagtaaactttaacttccatcacatcaaatatttgagcacatgcatagagtactaaatatatagactatttacaaaattaaaaacacaactagagaataatttatgagacaagttttctgagcctaattagtctatgattggacactaattgtcaaataaaataaaaatactataatacctattaaactttaataccttcgaccaaacaagcccttacagggtttcaaatatgtatataaaattattttcgttaagctttcatattaaacttctcattgttgtctcattaccatctttccctgcaaaatgtgaaaacaaggtggataaatacatgaatccacatctgttctcacccctagtatttagtaaaaggaaatagtgtactctctcaagtacaaataataatgtttcttgacttcaacacctctaacacaaaatcgtaactaatattatttgtgtaataatatatatctataaaagaacatgttgcctctctctagaaaagtctacctcttgatgtcattttccaaatatcaaaactcgatacacaaaagaattgatttagaaccaaagattaaaatgcctgactacatgatgaaacctgaaaacattgttctattattagtgactgaagggagtaatatccaacagtaacttcttgttgcgaagattagtgttgtacgcaaaaagaaatatccatattcctccatataaaggagatgatgagatcacagtgattttctggttcagtcaaaaccagtggcaaagttgggtagggaattgaagcatgtgaacccaaaaatttactgattcgtcttcgtcttgacgacgttaacgtcgtcgcatctgagaaacttccattcgattgactaataagccctgataataaatataccacacccaaagagcttcatcactactctctcaatctctctccctctcgtctacatggttcattcattaaactttgcgacaacatgggagcagcagtagagcacaggacgtcgtagacgtacggtcactggcggcgtccatggattcaagctcacagcccggcgcaatgtatgcatctctctctctctctctctctctctctctctctctctctctctctctctctctctctctctctctacatcatcgtttgggggatgaatcaaatggggctgccaattatcaaggaatgaatggtttttgttcaccctccttatattagtctttctctctacgctgtgtttggtgcgtttgccttaaaccacactcggtgtattaggggttggcaacttatcatagctttggttctcatgcatgcatgtatggttcatcatgtttttgtcaaattttcatgtagcaacatattgtcctccgtccacaacagataagctgatcctgctagtcatagctgctatatacagatcagcttattaagtttgcatcattgtagaagcaaaagtaattaagcacccgggcggcagacatgttacgtacgtatataacaggttgttgttatgcgtgttctaatgttccttggcacaacaactgtagtgatacatgcagagggagcggaggaggaggagatagaaaccaaagggaggaggacgcggcggcggcggcggcggcagaggccggctacggcaggcagctggtgattcccgaggacgggtacgagtggaagaagtacggccagaagttcatcaagaacatccagaaaatcaggtacttgctccgttcgatccaacatatgcatacgtagcatttttggcatcgagattgatctcgagctctcaaataaagctagtgcaaacttgatcacatataccattttttcgtggtcaaatctcgtttcccgccatacgcgtgtacatcagattaatcaatagctcgacgttgaccaagcttgttgacttgttcatcttcgttcctgtgcatcaaatcgttttattaattaattgagtcgatgtgacgcccatcgatcgatcactggtataatggaatgtatgggttgcccgccgtccccgtgcatatatgcatacgtgcaatgctctgctgccagatcttatctttcgaagaagaatcaacggaagaataatatcctcgctttattatattatatattgataacggtcgaccaaataaagccctgatgatgacttgatgagcaaactgcacaagtgtgttttgcattgcatgccaactgatgataccaccgtacgtgggtggtccatgatgcatgtgtgtgatcaaaatccaacaatggcgcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcagggcggcggcgcctccgacttcaacagatacgagctgggcgcgcagtacttcggcggggccggccggtcgcattga

SEQ ID NO: 4 - DNA, 3437 bp, Sorghum propinquum
taagatgactctattttttatcaataagcactttgtactatgattaagacaaaaggaagagaggggacaagaattacaaactatacttaggggttgtttgaatttcagtcatagttggtcacaactcagatgtggtgagacacactctatgatgagaataatgagatctgtttggttctcttctcacctaggctacatcgcatctggagcgagagacaggctagccacagcctggtctggtgcatgcacctgcacttgtttggttttgctctttgttttgagccactccagccatgtctcggaaagatattgttttgttggtctttggcttggcaccagtgctctctcacgcgtacaggcacacgctctcttttggctccacgcagccatgtgttggctaaaaatgattttagaatccatttcccatgagcctgagatggttgcacgcactataggtctaaccctggtagcactttaggtaaccaaacaccttaagcctgcatcccaagagccaggccagtttggaaactggacaaccaaataggcctctaatgaatttgatgtgttgtattctgtgggtgtctagcactcttcaccaactaaacactgataaaaaaaagttatggtgtgcgatgccttagtgtggcatagcaagtgaaggccgggaaccaaacatgcttttactctttcatatcttaggccatgtttggtttgtcgtagtaaactttaacttccatcacatcaaatatttgagcacatgcatagagtactaaatatatagactatttacaaaattaaaaacacaactagagaataatttatgagacaagttttctgagcctaattagtctatgattggacactaattgtcaaataaaataaaaatactataatacctattaaactttaataccttcgaccaaacaagcccttacagggtttcaaatatgtatataaaattattttcgttaagctttcatattaaacttctcattgttgtctcattaccatctttccctgcaaaatgtgaaaacaaggtggataaatacatgaatccacatctgttctcacccctagtatttagtaaaaggaaatagtgtactctctcaagtacaaataataatgtttcttgacttcaacacctctaacacaaaatcgtaactaatattatttgtgtaataatatatatctataaaagaacatgttgcctctctctagaaaagtctacctcttgatgtcattttccaaatatcaaaactcgatacacaaaagaattgatttagaaccaaagattaaaatgcctgactacatgatgaaacctgaaaacattgttctattattagtgactgaagggagtaatatccaacagtaacttcttgttgcgaagattagtgttgtacgcaaaaagaaatatccatattcctccatataaaggagatgatgagatcacagtgattttctggttcagtcaaaaccagtggcaaagttgggtagggaattgaagcatgtgaacccaaaaatttactgattcgtcttcgtcttgacgacgttaacgtcgtcgcatctgagaaacttccattcgattgactaataagccctgataataaatataccacacccaaagagcttcatcactactctctcaatctctctccctctcgtctacatggttcattcattaaactttgcgacaacatgggagcagcagtagagcacaggacgtcgtagacgtacggtcactggcggcgtccatggattcaagctcacagcccggcgcaatgtatgcatctctctctctctctctctctctctctctctctctctctctctctctctctctctctctctctctacatcatcgtttgggggatgaatcaaatggggctgccaattatcaaggaatgaatggtttttgttcaccctccttatattagtctttctctctacgctgtgtttggtgcgtttgccttaaaccacactcggtgtattaggggttggcaacttatcatagctttggttctcatgcatgcatgtatggttcatcatgtttttgtcaaattttcatgtagcaacatattgtcctccgtccacaacagataagctgatcctgctagtcatagctgctatatacagatcagcttattaagtttgcatcattgtagaagcaaaagtaattaagcacccgggcggcagacatgttacgtacgtatataacaggttgttgttatgcgtgttctaatgttccttggcacaacaactgtagtgatacatgcagagggagcggaggaggaggagatagaaaccaaagggaggaggacgcggcggcggcggcggcggcagaggccggctacggcaggcagctggtgattcccgaggacgggtacgagtggaagaagtacggccagaagttcatcaagaacatccagaaaatcaggtacttgctccgttcgatccaacatatgcatacgtagcatttttggcatcgagattgatctcgagctctcaaataaagctagtgcaaacttgatcacatataccattttttcgtggtcaaatctcgtttcccgccatacgcgtgtacatcagattaatcaatagctcgacgttgaccaagcttgttgacttgttcatcttcgttcctgtgcatcaaatcgttttattaattaattgagtcgatgtgacgcccatcgatcgatcactggtataatggaatgtatgggttgcccgccgtccccgtgcatatatgcatacgtgcaatgctctgctgccagatcttatctttcgaagaagaatcaacggaagaataatatcctcgctttattatattatatattgataacggtcgaccaaataaagccctgatgatgacttgatgagcaaactgcacaagtgtgttttgcattgcatgccaactgatgataccaccgtacgtgggtggtccatgatgcatgtgtgtgatcaaaatccaacaatggcgcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcagggcggcggcgcctccgacttcaacagatacgagctgggcgcgcagtacttcggcggggccggccggtcgcattgacgcggggcgctagttcctaaaatattttgtaaaatttttcacattctcgtcacatcaaattttgcggcacatatatatatatatagagtactaaatatatataaaaaaataactaattacatagtttacctataatttatgagacgaatcttttgatcctagttagtcaataattaacaatatttgttaaatacaaacaaaattattactattcctatttta

SEQ ID NO: 5 - DNA, 600 bp, Sorghum propinquum
atggcggagccggggctcgagggcagccagccggtggatctgtccaagcacccctccggcatcgtccccacgctccagaatattgtatcaacagttaatttggattgtaaacttgacctcaaagcaatagctttgcaagcacgaaatgcggagtataacccaaagcgttttgctgcagtcatcatgagaataagggaacccaaaaccacagcactgatatttgcatcgggtaaaatggtatgtactggagcaaagagtgaacagcaatctaagcttgcagcaagaaagtatgctcgtatcattcagaaactaggttttcctgctaaatttaaggactttaagattcagaatattgttggctcttgtgatgtcaagtttccaattaggcttgagggccttgcatattctcatggtgccttctcaagttacgaaccagaactctttcctggccttatctatcggatgaaacaaccaaagattgttcttttaatttttgtttcaggcaagattgttttgactggagcaaaggtgagagaggagacttacactgccttcgagaacatctatcctgtactgacagagtttagaaaagttcagcaa

SEQ ID NO: 6 - DNA, 15552 bp, Sorghum propinquum
gtccttcttcctccggcacccataataaacaaaacaaactacacgatcgagatctcgccaggatttaatttgacacgtgcatggatcacgtacggtttgttggatcgtctccaacaataagacgaatgaactgatagtactatatacgcctacacccaccaacgtgcatggatcacacggttcaattagtttgtcttccacacgtgcatggaaccgtgagtcattcagaatcgtagccttaatttgatcaaccagtatgtccatccgttaaaatgctccactaaacatatattaatatttaagaaggtcggagttcacattcacatggagactactactcggagactactactcgctctgttttgtttttgtaagagggtgtttgggactgctctgctccatgttttccagctccgctccatgttttttagccaaacggtttcagcttcatgcactcaaggaaaaagggtggagttgtgagagcacctaaagaggtactccacaaactccagttttttttggagctgctccatggtagagtttgtaaagcagagtttgtggagcagtcccaaacaccttgacgaaagttttcaagaaatccaaaaagttttcaagattttttgtcatatcgaattttgtggcacatgcatgaagcattaaatatagacgaaaataaaaactaatcacacagtttgactgtaaatcgtgagacgaatcttttgaccctagttagtctatgattagaaaatatttaccacaaacaaacgaaagtgctacagtagcgaaatataaaaattttcacttctaaacaaggcccagctagcgctggctaaagggtaaaagaaaagaggcagcagcttcttggaacaagaccacgcaacgagggaacggttgctgacgtaagacaagtgacgtcagtcacggctccagccgcgacctggcgcgacattccctcctctccaaaccacgcggcccccgccccgctaacggccgtccaaggtttaggacgatcgcagagcgtgctttcaggtttgaatttgatcggcataaagtttccgtttgcttgaaatttgtatattcgtccttataaaattggtgtattatggccttgtttagttcctaaaattttttaagatttaccgtgacatcaaattttgtggtatatgcatagaacattaaatatagataaaatgaaaaactaattgtatagtttatctgtaatttgcaaaacgaatcttttaagcctggttagtccatggttgaataataattaccaaatgcaaacgaaaatgctacagtagtaaaatcaaaaaaaaacaaactaaacaaggcctatgcatgaaagctgagaagcggatcgttggattctacttcttttgttccaaattatatgttgttttaattttccctccaggagaagcaaacaagtcatttgtttgtttcagcttgcatattgtaacaacttataagatgactctattttttatcaataagcactttgtactatgattaagacaaaaggaagagaggggacaagaattacaaactatacttaggggttgtttgaatttcagtcatagttggtcacaactcagatgtggtgagacacactctatgatgagaataatgagatctgtttggttctcttctcacctaggctacatcgcatctggagcgagagacaggctagccacagcctggtctggtgcatgcacctgcacttgtttggttttgctctttgttttgagccactccagccatgtctcggaaagatattgttttgttggtctttggcttggcaccagtgctctctcacgcgtacaggcacacgctctcttttggctccacgcagccatgtgttggctaaaaatgattttagaatccatttcccatgagcctgagatggttgcacgcactataggtctaaccctggtagcactttaggtaaccaaacaccttaagcctgcatcccaagagccaggccagtttggaaactggacaaccaaataggcctctaatgaatttgatgtgttgtattctgtgggtgtctagcactcttcaccaactaaacactgataaaaaaaagttatggtgtgcgatgccttagtgtggcatagcaagtgaaggccgggaaccaaacatgcttttactctttcatatcttaggccatgtttggtttgtcgtagtaaactttaacttccatcacatcaaatatttgagcacatgcatagagtactaaatatatagactatttacaaaattaaaaacacaactagagaataatttatgagacaagttttctgagcctaattagtctatgattggacactaattgtcaaataaaataaaaatactataatacctattaaactttaataccttcgaccaaacaagcccttacagggtttcaaatatgtatataaaattattttcgttaagctttcatattaaacttctcattgttgtctcattaccatctttccctgcaaaatgtgaaaacaaggtggataaatacatgaatccacatctgttctcacccctagtatttagtaaaaggaaatagtgtactctctcaagtacaaataataatgtttcttgacttcaacacctctaacacaaaatcgtaactaatattatttgtgtaataatatatatctataaaagaacatgttgcctctctctagaaaagtctacctcttgatgtcattttccaaatatcaaaactcgatacacaaaagaattgatttagaaccaaagattaaaatgcctgactacatgatgaaacctgaaaacattgttctattattagtgactgaagggagtaatatccaacagtaacttcttgttgcgaagattagtgttgtacgcaaaaagaaatatccatattcctccatataaaggagatgatgagatcacagtgattttctggttcagtcaaaaccagtggcaaagttgggtagggaattgaagcatgtgaacccaaaaatttactgattcgtcttcgtcttgacgacgttaacgtcgtcgcatctgagaaacttccattcgattgactaataagccctgataataaatataccacacccaaagagcttcatcactactctctcaatctctctccctctcgtctacatggttcattcattaaactttgcgacaacatgggagcagcagtagagcacaggacgtcgtagacgtacggtcactggcggcgtccatggattcaagctcacagcccggcgcaatgtatgcatctctctctctctctctctctctctctctctctctctctctctctctctctctctctctctctctacatcatcgtttgggggatgaatcaaatggggctgccaattatcaaggaatgaatggtttttgttcaccctccttatattagtctttctctctacgctgtgtttggtgcgtttgccttaaaccacactcggtgtattaggggttggcaacttatcatagctttggttctcatgcatgcatgtatggttcatcatgtttttgtcaaattttcatgtagcaacatattgtcctccgtccacaacagataagctgatcctgctagtcatagctgctatatacagatcagcttattaagtttgcatcattgtagaagcaaaagtaattaagcacccgggcggcagacatgttacgtacgtatataacaggttgttgttatgcgtgttctaatgttccttggcacaacaactgtagtgatacatgcagagggagcggaggaggaggagatagaaaccaaagggaggaggacgcggcggcggcggcggcggcagaggccggctacggcaggcagctggtgattcccgaggacgggtacgagtggaagaagtacggccagaagttcatcaagaacatccagaaaatcaggtacttgctccgttcgatccaacatatgcatacgtagcatttttggcatcgagattgatctcgagctctcaaataaagctagtgcaaacttgatcacatataccattttttcgtggtcaaatctcgtttcccgccatacgcgtgtacatcagattaatcaatagctcgacgttgaccaagcttgttgacttgttcatcttcgttcctgtgcatcaaatcgttttattaattaattgagtcgatgtgacgcccatcgatcgatcactggtataatggaatgtatgggttgcccgccgtccccgtgcatatatgcatacgtgcaatgctctgctgccagatcttatctttcgaagaagaatcaacggaagaataatatcctcgctttattatattatatattgataacggtcgaccaaataaagccctgatgatgacttgatgagcaaactgcacaagtgtgttttgcattgcatgccaactgatgataccaccgtacgtgggtggtccatgatgcatgtgtgtgatcaaaatccaacaatggcgcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcagggcggcggcgcctccgacttcaacagatacgagctgggcgcgcagtacttcggcggggccggccggtcgcattgacgcggggcgctagttcctaaaatattttgtaaaatttttcacattctcgtcacatcaaattttgcggcacatatatatatatatagagtactaaatatatataaaaaaataactaattacatagtttacctataatttatgagacgaatcttttgatcctagttagtcaataattaacaatatttgttaaatacaaacaaaattattactattcctattttataaaaaaaaaattcaagtaaacaaggcctaggttgacaaaccgacaagaaaggccggcggcgttgcgtcacgtacgcatgcatcagctcctgtacgtgctggcctctgctggctgccgctgcatcgatcgatcgctttcgctgcgcaccggagggcaacggcaggtgctgccggtgccggttgacgccttgcgccggcgcaacatgatgttgagtgcggactaattgttgctgctccggttaactctctggtctagttctagtgtacggtactattaggacgatggtgcataattgtaattttcatattgtatatggataaaaaaatatttagctgaaagtggaaactagcaccgtcgctattatgttttgttttttgcaactctaaagtgtaaacttgtgctctagtagtcgaaagtctccagagttgggttcgaggccctggtcacccgggcttacattgcatcgcctctgaactgaatgcgacactcgagacctagctttatcagtgggataaacctaattcgtttagtcagctttaacattcaatcatttgtagatagcaaggcatcaatgggtaacgaacgccgcactgtatcccctaacctctgccgacaactgatcactgcaacggctgggcatccattaccaacaagttggcaacattaataaaatgttttcgattgaggaaaacggcaaacacagttccatgcgatacaagacagctcgttcgccgagcaatctttccagatacgttaataggcattcttatacagtgcgtagaattcaaattattcatcctagcatgcaacatcgaaaaagtaaaagaaccaagtgcaggtacatttggatacagaaacaagtctactgcgtggtcgactgaccggttcctccatacagtgataaccaacaagattattcccggtgtcctctacgatacagcatctcaaatacaacagataacttacaaccagtcacacagtcccgtcagtagtcagtacattgccccagttacctacagtgccagccttttcatcatcgcacagcactgaaagatactcagaaaagactttaatagactcgtgtctcaaagacaaagtagggcaaaatttatctactcttgttagcactcaagttaaccacatgggacacaaactactcaaactgaagcatgataggtgtccgtgttcaccagggcctacccaaatggacaaatctgacaagtccatcagctaccacaacaaacccacccaaccatgacacaccgaggctcacagaaattacaggatgctataagttccgccagactttttatgtacagttagaatttatggtcacacaaaaaacctcaaggatgcttgtaattagaagaacgtgaccttcacttgggtcatctgcaaagagggaaccagaaggaaaagattagttttaaatagttaattctagtactgcacacaccgacacgagttataaacaatataaacaatccatttggaatacagaaatttcacagaaatcatgtacaattccaagggaatcggtccattttcacaggaaaacacaggaaacagggggatcccacattccaaaaggggcttaacgagagaaggaattatcccctcaggcagctatttacatgccatgacatctgatttgaataactagaataccataataaaagtttgtttcgaaaacacagtagaaaacatggttccaacattttactatcaagtctaacaacaaataacatataggtgcccagtcccacacatgttccaaaatgagtacaagacatagtgaacatagtcaacagaacaagagaatctcaattgtaggaagagtcatgcatgcactactgaagcatgataaaaagaactacataccattgctgaacttttctaaactctgtcagtacaggatagatgttctcgaaggcagtgtaagtctcctctctcacctacaaacaatcgactatgaaatgaaggagaaagataagcaaatcgcagtataattaagcatgagcacgaaatgacaactaacctttgctccagtcaaaacaatcttgcctgaaacaaaaattaaaagaacaatctttggttgtttcatccgatagataaggccaggaaagagttctggttcgtactgtaaaacaaattaaaaatgtcattatccaaagaatgcagacaaaaaagggtaaaagaattactgtgatgttaaaataagccataattggacatacacttgagaaggcaccatgagaatatgcaaggccctcaagcctaattggaaacttgacatcacaagagccaacaatattctgaatcttaaagtcctggcatagaacagtaacttagcaactgatgtacaaattgttcaaagtacaggtcaatgtacacaagtatgaaaatagttaccttaaatttagcaggaaaacctagtttctgaatgatacgagcatactggaaatacagacaggggttagaattccaaagcctctcagtaaactagatccaacttaaataaaatggtagcaagccatatggcacctttcttgctgcaagcttagattgctgttcactctttgctccagtacatacctggtcatagaaaattatcggttgcttgcttcagcactagaacacttatgatggattgatacaaaattgtagttctatatgaaagaaatgcagttctagtaaactttcttcatttggaagaaaagtatttgacacatcaatacatttaattaatattgaatatgacaaccaagaaactctacaatactgaacattgatccaaataaaatcccaagtaaaaaacccaccgacatatatcatctggtaagggaaaaatagatttgcctagggtaggctagagagggtaagaactttattctccaatatttgatgattgagagaggtagattaggacacagaaaaacaaaaagattagcctttctatcttttgacagcacagcaccaaggcaacaaaacatgtcaaaaaaaaaagatcaaatctgtttacataaaaaacatgcaaaatccttgaaaattgacagtataagacaaaagatgttgatgacataccattttacccgatgcaaatatcagtgctgtggttttgggttcccttattctcatgatgactgcagcaaaacgctgtttacagataaaaaagtcaaatacgaaatataatgacagaaaacttagcaaaattcaggttgctacactgtatcatcataactgagaaagattgcattcaatagaatgcctaaaagagcaaacaagtcatatataagctaaaaatttagaacttgtttgtcaaagaatattgtggttattcacaggacaagcaggatatgagcatccatctggttaaaaactaaccgtgcgcatctcatatcccaggccatccattagttattagcacaaagctatttgaactcatggacaagattgtacatcattacaaaggatcaacatactttatatatccataaatcttccactagataaaaccaccagtaaataccgtgcagccattgctttgaggtaatcactatacctttgggttatactccgcatttcgtgcttgcaaagctattgctttgaggtcaagtttacaatccaaattaactgttgatacaatattcctgtcatgaaaaaatggcacgtcaaacagaccatgatcaaagaactgcagtaaacatgtgaattttgttttgtaaatccaacatagggttcttattataagtttttagcattgaagagacactacaagatgattttcattgttctttttttatatgatagtgtgtgctattaatttcttcttcatgccaatttccaacatgtacaatcataacaaatttaagactaacattcaagataacctaccctataatggttggatcataaaatctttgtatcaatcaaagtcatttcaggactcaatatggcactaataagcccatagcacttaataatgaaatcacctgcagaaaaatcttacacctaaatcatactaaaaatcttccacaaaagctagttaggttacttctggtttggggacggagtgggatggaatggtcatgtccctattttttggacgggattgacccagaccttgtttggttggacggataggttcattccaatttttgtttggttctaaggatatggtgggatggaacccgctggagttttaactccattagacacaataatccatggccgcaccagccattgtctctacacctattcttgttgtcttcttcgggtgagcaaagcctgattcccaagattttgtaccacagtcactcaacatctcacagctccggtgcccaacagctgggcactaccaccgcccaagagcttggccaacccattcgcccaagatctcatgcagagatcttggcattgccaccatcagagatgctcaacctgccccaccagagatctcatgtggccagaggaggtaattggacccgctccttcccatgctggagctcaccccactcctctcatatatcgtcggcgctaacccagtgcgctgcatattctccaaacatctcctctcctctggttgccttgagcttggagcttccacatgcccgcgcccctccttttgaccacgcttgcaccaggcaatgcaaagatggcgtgcaacacggtccgcaaggaatggcttcatccactcgcttcaaggggaccgagctgtccaagtatttcaggaatatgccactgcaaaaatgaccccatccctagctcctcccaaccaaacactgctgaaaaaggattggcccatcccgtctggaacgtccctcaatccaaaccaatgcatttaaccctccccagggtatgagatatcgaaacctcagtccgtgaggctgactgtttatcatattacacaatttatgcaccaaccagtcaaaacatggaatggaaatatggtaagaagagattatgcttgctgcaactattacgccaagatgacaaacttcaataaggaaatagatctcctctccagtttggccctctctcgttctcccaagtttcatacctgaaatcaaccctcggagagaggatgacaactaaataattcccaccaaagccccaactatttaagacaatattagctcgtttcgatgcacccagcactgggaagctgaacaaaaacacggcataaaccaaccacaccaccacccacaagacagggaggcaccccgctggccagaaccaagccttggcagctccacagcacacccaagcacccatccgccgggcggcgggaccctagcacgtacggtacgggatctctccggaaccccgaatccccgacgacccagatccgggacttactggagcgtggggacgatgccggaggggtgcttggacagatccaccggctggctgccctcgagccccggctccgccatccgaaccacgcacgcgacctcggcggggctccgcgccgcgaatccgggctcaatccggggccgaaatgggcgggaaaggagcgcgcgcgtcaccggttcgagggggaattcgaaatccgggtcttttatagagatcgggagaggagttggggaggagggaaagcaaggggaaggagagctagggttatctgtctcgcgagggggagtcggggacagcgcgggcggcgtgagaatgcggggggaagagggggaggtcgtctggtggtgggaggtagatgcgtgcgggagttggggttgtatcggtggacggggagcaggcggtggatggcgagtgcttggcttttgtaggggaacagggtgcaccggctgtggccggttaccacagggcgcggtttgcccacgcgctggttcgagttatacaaactgacctgtgggtcatagcatgcggtggggcccggtgtcggtgtgtgggtatgatgcgcgttcgacggccattaatcaagaatttctcctgctcgcaaatcgcactagcaggttacgaacgcaccgagaagatcgtactatggttctttgaaagaaaattattatgaattatgaaatgatgaatgatgaactatactaatcggactgtttgaattattgtgatggatcattttcgttcgagtgggaaatcatggtcaccaaaaagctggtaagagagagagattatatataatcgagtgttttagttatgtttagttcataattaacttattttagctaattattataaccatagtggatccaaacaggcctgactagtgactacttgagcattcgcgttacgtcactgttgcagtgcacattcattcgtattaactaaaacatcttgcattagagcttccctgatgcaccacggtggcgtgctgtcgcagtgaccaccttagctttagacttccatgtcataggaagttaagcctcgtagagtctcatgttctcttgcagagaagatcatggcctcatctgacaaaaattaaaagcaacggctatgaacaagtattatagtgagctgtaagctgactaaatgctgaggtgggggagagaagaaatgagagagaagagaagcaggctataagggcactcacaatgcaagactctatcacagagtccaagacaatttattacatattatttatggtattttgctgatgtggcagcatatttattgaagaaagatgtagaaaaaataagactccaagtcttatttagactctgagtccacattgttcgaggtaataaataactttagactctatgatagagtctgcattgtgagtgccctaagcttatagccagcttaagcacaggaaccaagaaactttgtgagagataagtaggccatatattaataatgaatagttaactattgtatgtgtgggttgggagaaggctgtaaagaaccttagggcactcacaatgcaagactctatcacagagtccaaaacaattaattagatattatttatggtattttgttgatgtggcagcatatttattgaagaaagaggtagaaaaaacaagtctccaagtcttatttagactctaagttcatattgttcgagataataaataactttagactctatgatagagtctgcattgtgagtgcgcttacaccagcaagtggcctgtattattaaacttgctctaagtagcgcgatgtggtgagaatagtgactctaggctattgggaccacgtctggttcgtgcatttggctccaaattgtctcagcgattgacggtcggaccccagacaagccacatgcagctttgcattgagtaaaaacggtggttttaacttttaatccaacggacgtacgtggatggtcaccttttttcctagagctaacgctactaggtgcccgtgttgcgacgactcctccacaatggtgaacatcgatgtgtcagtaagcatgtcagtgagcatcggttcataagagagctgcaatgtctaagcatcatgtgggaccacccaaatgaataaacaaacaaggagacattgcaatgcctaaacatatcattgagcattagttgagactcgacctctctcactatgtgcaatagtttttttatgttgcaccgtggaaagtagaagcctcgatgccgcgcaaaaaaaattcagcatcacaccccaaatgtgatgcctcgaggcgagaagccaaaatatgtgcattggtaaaactatacgttatgcgtagtcttatatataaaatgttagcaaaaaattctttcattttagaatggagatagtaggcaataagaccagtacaaaacggacataaatctaaaacaaatattgtttgagagaaaagatctaaaatcaatccaagtagaagcaagcatcatatgtgacataataagagattaataatcctaaaatgagtgtacatgtcttgcatcaatttatgaaactcgaattatctgtctcccagagcacaagccaatgctactcataacctattacatatacgtcaatcttttacagaacttgtgatcatctttatatatgatcatcatttaacgatctgcgggactagtaggctatcagaagcaataaccttcggttgtttcagatggacacgaatgtgcatcaccagtttacagctctgtatacttcacctaataactgaacattctgagagaatgaactatttgtggctccttgatgaggcccagcatgtttaccttttaggttcccttaggttaaacactaaatcttcatgatggaaggtgtttgcctgaactccaagacagcaaggttttctctatacttctttacttcggccaccattctgttgtacgattcagggtatttgcaaaaaatcacgattttgattcagctccctggctcgtgcctgcaatgtcaacatgatcctttacaaatgttcgaaggcatccattaattacccgaggggcaccaccatcacaaaatcgctttgccagatctactgcctgaaagacaagggtcgagagacttttattctactagtactcaaaaatggaaagagtaatagctataagaaaacatgcaggtgctagatgcataaagtcaaaatatgaagaaaaacaagtaattgggagaaaataagcacctcattaatgacaactttgtgaggtgttccttttgatgtcatctctgccatagcaatatgtagaatgcagagctcaagtatccttgccacaggctcatcctgccatgaatttttccatgtatcaacagcaggttatgccataaaacaagacagcaaaataataaatactaaaatatttaaccagtttaaagatcaggtagattataaactgatgaaaggaaagtaatatattgtgtttcatatttttctaatttttactttaaaaaacatctgagctatggtagtagaaacaaatatagaaataaagcgattcagattaaggaaggtgcattcttcagattctgtatcacttcctcatccttggggtggccaacagaaataactaattaactatgctggaaaattaagtagtgtaataaggccataagtctaaaataacaatgggagatctcaatatttcactgcatgccaaaagataaggcaggaaataatctttgatggtcacatgcttttggtatgcatcagagtgattgttcactagttcagtgtagtgaaaaacagttgtgtaatatacagaataaggatacgttcaaatcaaactgataaccatatataaacatcttctggtatgcattgttcactagttcagtgtagtgaaaaatggttgtgtaatatacagaataagtatatgtttgaaccaaactgataaacatataaacagcttgatgcatatcgcagggatttgatgaatcaacatagaatattaggaaaaggtatctaaccttccaagcctggggaattattttgtcaatgatatctacatgcttatcccatccactagcaacagccactaaaagttccctggacaacctgtacttgaaaagtatataattaggaatgtaagagcagcaggactaaatattgaacaggaaattaaattttatcatatatcagaacagtgtatcgatacctaatgcctttagtggaatggggcaagaaggaaagtataccgtaagacgaagttgttgtacaccagttttggaggagctgaaagtacatcttcttctgaatatgaaagaaaaacatgtcaaattctttgcagaagaataaccaaacattaatggaacatatttacacaaaaacaaatctatagttactcagctgatttcacaacagactaaggaagaaaatgtatatggttaatatgactatatgagctgtttagcacgcatcgtaaggatacgtttattgtgctgaacgagatagatgccactgggctgctacaaaagatgcatgctaacgaaggtgaacagttttcagcatgtcgattaaaagtgtaatcaatacatagcttggtaaaatatatcaaaatttactgccgcttagagtgatggattatggtatagctctcttaaaactcagtctgcaacccccccccccccccaaaaaaaaaaaaaagacacacaacccccttagatcttgacgacctagcctgactaggtagcacctaggcattagccactataccgaatcaagagttaggtgccacgcagctgcttacctagcacattgcgtttttttaagccaaagcactgcgttaactgttctagtttgacggtctgaaattcacagcaccaacttgaaattgctctagcatgccctccagtttttatatacatgaaaataggcacacgcccacaataaaaaaaaaagaaaattggcctaagttcaataatgtatttatggaacaaccaatgatccattgctctctttactttaggaaatcagaatcatagatatatgacataaagtttcaaaacttagactgaaacccaccataaaatttatttaaacaggaatcaactagattttctggtggttgtatgtttcagattgaccgaaggataaccattaaaagactgctataatggaattggtacctaactgaacttgtgctctttggaatcttctggatatagagatattccatctcaaaattgtgaaaaaaagatggacatatgtccaatttaccaacaacaatctactactccagctgtaacagcgttaacatataggaagtag

SEQ ID NO: 7 - DNA, 303 bp, Sorghum bicolor
atgcccgaggacgggtacgagtggaagaagtacggccagaagttcatcaagaacatccagaaaatcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcacggcggcggcgcctccgacttcaacagatacgagctgggcgcgcagtacttcggcggggccggccggtcgcattga

SEQ ID NO: 8 - DNA, 844 bp, Sorghum bicolor
atgcccgaggacgggtacgagtggaagaagtacggccagaagttcatcaagaacatccagaaaatcaggtacttgctccgttcgatccaacatgcatacgtagcattttttgcatcgagattgatctcgagctctcacataaagctagtgcaaacttgatcacatataccattttttcgtggtcaaatcgtttcccgccatacgcgtgtacatcggattaatcaatagctcgacgttgaccaagcttgttgacttgttcatcttcgttcctgtgcatcaaatcgttttattaattaattgagtcgatgtgacgccgcccatcgatcgaacactggtataatggaatgtatgggttgcccgccgtccccgtgcatatatgcatacgtgcaatgctttgctgccagatcttatctttcgaagaagaatcaacggaagaataatatcctcgctttattatattattgataacggtcaaccaaataaaaagccctgatgatgacttgatgagcaaactgcacaagtgtgttttgcattgcatgccaactgatgataccgtacgtggggtggtccatgatgcatgtgtgtgatccaaatccaacaatggcgcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcacggcggcggcgcctccgacttcaacagatacgagctgggcgcgcagtacttcggcggggccggccggtcgcattga

SEQ ID NO: 9 - DNA, 2867 bp, Sorghum bicolor
ttggtcaactcagatgtgctgaggtctgtttggttctcttctcacctaggctacaccgcatctagagggagagacaggctagccacagcctggtctggtgcatgcacctgcacttgtttggttttgctttttgttttgagccactccagccatgtctcgaaaagatattgtttggttggtctttggcttggcaccagtgctctctcacgtgtacaggcacacgctctgttttggctccacacaaccatgtgttggctaaaaatgattttagaatccatttcccatgagcctgagatggttgcacgcactatgggcctaaccctggtagcactttaggtaaccaaacaccttaagcctgcatcccaagagccagtttggaactggacaaccaaataggcctctaatgaatctgatgtgttgtattctgtgcctgcctagcactcttcaccaactaaacaccgataaaaaaaagttatggcacgcaatgcctgagtgtggcatggcaagtgaaggtcgggaaccaaacatgcttttactctttcatatcttaggcctgtttggttcgtcgcggtaaactttaacttccatcacatcgaatatttgaacacatacatagagtactaaatatagactatttataaaattaaaaacacaactagagaataatttatgagacaagtatttttagcctaattagtctatgattggacactaattgccaaataaaataaaaatactacaatacttgttaaactctaataccttcaaccaaacaagcccttacagggattcagatatgtatataaaattattttcgttaggctttcatattaaacttctcattgttgtctcattaccatctttccctgcaaaatgtgaaaacaaggtggacaaatacatgaatccacatctgttctcacccctagtatttagtaaaaggaaatagtgtactatctcaagtacaaataatgatgtttcttcaacacctctaacacaaaatagtaactaatattatttgtgtaataatatatatctataaaagaacatgttgcctctctctagaaaagtctacctcttgatgtcattttccaaatatcaaaactcgatacacaaaagaattgatttagaaccaaagattaaaatgcctgactacatgatgaaacctgaaaacattgttctattattagtgactgaagggagtaatatccaacagtaacttcttgttgcggagattagtgttgtacgcaaaaagaaatatccatattcctccatataaaggagatgatgagatcacagtgattttctggttcagtcaaaaccagtagtgtcgaagttgggtaggacagcatgtgaacccaaaaatttactgattcgtcttcgtcttgacgatgttaacgtcgtcgcatcagagaagcttccattcgattgactaataagccctgataataaatataccacacccaaagagcttcgtcactactttcaatctctctccctctcatctacatgtttcattcattaaactttgcgataacatgggagcagcagtagagcacaggacgttgtagacgtacggtcactggcggcgtccatggattcaagctcacagcccggcgcaatgtatgcatctctctctctctctctctctctctctctctctctctctctctctctctctctctctctctacgctgtgtttgatgcgtttgccttaaaccagctttggttctcatgcatgcatgtatggttcatcatgtttttgtcaaattttcatgtagcaacatatattgtcctccgtccacaacagataagctgatcctgctagtcatagctgctatatacagatcagcttattaagtttgcaggttgttgttatgcgtgttctaatgttccttggcacaaaaactaactgtgtagtgatgcacgcagaggcagcggaggaggaggagagagaaaccaaagggaggaggacgaggcggcggcggcggcggcggcagaggccggctacggcaggcagctggtgatgcccgaggacgggtacgagtggaagaagtacggccagaagttcatcaagaacatccagaaaatcaggtacttgctccgttcgatccaacatgcatacgtagcattttttgcatcgagattgatctcgagctctcacataaagctagtgcaaacttgatcacatataccattttttcgtggtcaaatcgtttcccgccatacgcgtgtacatcggattaatcaatagctcgacgttgaccaagcttgttgacttgttcatcttcgttcctgtgcatcaaatcgttttattaattaattgagtcgatgtgacgccgcccatcgatcgaacactggtataatggaatgtatgggttgcccgccgtccccgtgcatatatgcatacgtgcaatgctttgctgccagatcttatctttcgaagaagaatcaacggaagaataatatcctcgctttattatattattgataacggtcaaccaaataaaaagccctgatgatgacttgatgagcaaactgcacaagtgtgttttgcattgcatgccaactgatgataccgtacgtggggtggtccatgatgcatgtgtgtgatccaaatccaacaatggcgcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcacggcggcggcgcctccgacttcaacagatacgagctgggcgcgcagtacttcggcggggccggccggtcgcattga

SEQ ID NO: 10 - DNA, 603 bp, Sorghum bicolor
atggcggagccggggctcgagggcagccagccggtggatctgtccaagcacccctccggcatcgtccccacgctccagaatattgtatcaacagttaatttggattgtaaacttgacctcaaagcaatagctttgcaagcacgaaatgcggagtataaccccaagcgttttgctgcagtcatcatgagaataagggaacccaaaaccacagcactgatatttgcatcgggtaaaatggtatgtactggagcaaagagcgaacagcaatctaagcttgcagcaagaaagtatgctcgtattattcagaaacttggttttcctgctaaatttaaggactttaagattcagaatattgttggctcttgtgatgtcaagtttccaattaggcttgagggccttgcatattctcatggtgccttctcaagttacgaaccagaactctttcctggccttatctatcggatgaaacaaccaaagattgttcttttaatttttgtttcaggcaagattgttttgactggagcaaaggtgagagaggagacctacactgccttcgaaaacatctatcctgtactgacagagtttagaaaagttcagcaatgt

SEQ ID NO: 11 - DNA, 14684 bp, Sorghum bicolor
tcttgcagtcgatctcgtcctagctactttggcatgcaggcaggcaggagagatctaccaaaagagtccttcttcctccggcacccatataataaacaaaacaaactacacgatcgagatctcgccaggatttaatttgacacgtgcatggatcacggtttgttggatcgtctccaacaataagacgaatgaactgatagtactatatacgcctactacacccaccaacgtgcatggatcacacggttcaattagtttgtcttccacacgtgcatggaactgtgagtcattcagaattgtagccttaatttgatcaagcagtatgtccatccgttcaaatgctccactaaacatatattaatatttaagaaggtcggagttcacattcacatggagactactactcgctctgtttctaaatgtttgtcgttttcgcttctcgagaaataattttaactaaatctatattataaaatgttaatatttaagatacataattagtattatttgatagatatttgaatctagtttttttaataaatttatttagagataaaagtgttacacgtattttctaataaattatttagagataaaggtagtaccgcacgatgcaaaaaaaaaaacccattaactgcacaggcatgatgctggaagcgtacgccaaatattacctagctagcgctggctgaagggtaaaagaaaagaggcagcagcttcttggaacaacaccacgcaacgagggaacggttgctgacgtaggacaagtgacgtcagtcacggctccagccgcgacctggcgcggcccccgccccgctaacggccatccaggggtttaggacgatcgcagagcgtgctttcaggtttgaatttgatcggcataaagtttccctttgcttgaaatttgtatattcgtccttataaaattggtgtattataaaatttgtttagttcccaaaatttttcaagatttaccgtcacatcaaattttacggtacatgtatgtaacactaaatatagataaaataaaaattaattgcatagtttatctgtaatttgcaagacgaattttttaagcctaattagtccaaagtctgtttggtcaactcagatgtgctgaggtctgtttggttctcttctcacctaggctacaccgcatctagagggagagacaggctagccacagcctggtctggtgcatgcacctgcacttgtttggttttgctttttgttttgagccactccagccatgtctcgaaaagatattgtttggttggtctttggcttggcaccagtgctctctcacgtgtacaggcacacgctctgttttggctccacacaaccatgtgttggctaaaaatgattttagaatccatttcccatgagcctgagatggttgcacgcactatgggcctaaccctggtagcactttaggtaaccaaacaccttaagcctgcatcccaagagccagtttggaactggacaaccaaataggcctctaatgaatctgatgtgttgtattctgtgcctgcctagcactcttcaccaactaaacaccgataaaaaaaagttatggcacgcaatgcctgagtgtggcatggcaagtgaaggtcgggaaccaaacatgcttttactctttcatatcttaggcctgtttggttcgtcgcggtaaactttaacttccatcacatcgaatatttgaacacatacatagagtactaaatatagactatttataaaattaaaaacacaactagagaataatttatgagacaagtatttttagcctaattagtctatgattggacactaattgccaaataaaataaaaatactacaatacttgttaaactctaataccttcaaccaaacaagcccttacagggattcagatatgtatataaaattattttcgttaggctttcatattaaacttctcattgttgtctcattaccatctttccctgcaaaatgtgaaaacaaggtggacaaatacatgaatccacatctgttctcacccctagtatttagtaaaaggaaatagtgtactatctcaagtacaaataatgatgtttcttcaacacctctaacacaaaatagtaactaatattatttgtgtaataatatatatctataaaagaacatgttgcctctctctagaaaagtctacctcttgatgtcattttccaaatatcaaaactcgatacacaaaagaattgatttagaaccaaagattaaaatgcctgactacatgatgaaacctgaaaacattgttctattattagtgactgaagggagtaatatccaacagtaacttcttgttgcggagattagtgttgtacgcaaaaagaaatatccatattcctccatataaaggagatgatgagatcacagtgattttctggttcagtcaaaaccagtagtgtcgaagttgggtaggacagcatgtgaacccaaaaatttactgattcgtcttcgtcttgacgatgttaacgtcgtcgcatcagagaagcttccattcgattgactaataagccctgataataaatataccacacccaaagagcttcgtcactactttcaatctctctccctctcatctacatgtttcattcattaaactttgcgataacatgggagcagcagtagagcacaggacgttgtagacgtacggtcactggcggcgtccatggattcaagctcacagcccggcgcaatgtatgcatctctctctctctctctctctctctctctctctctctctctctctctctctctctctctctacgctgtgtttgatgcgtttgccttaaaccagctttggttctcatgcatgcatgtatggttcatcatgtttttgtcaaattttcatgtagcaacatatattgtcctccgtccacaacagataagctgatcctgctagtcatagctgctatatacagatcagcttattaagtttgcaggttgttgttatgcgtgttctaatgttccttggcacaaaaactaactgtgtagtgatgcacgcagaggcagcggaggaggaggagagagaaaccaaagggaggaggacgaggcggcggcggcggcggcggcagaggccggctacggcaggcagctggtgatgcccgaggacgggtacgagtggaagaagtacggccagaagttcatcaagaacatccagaaaatcaggtacttgctccgttcgatccaacatgcatacgtagcattttttgcatcgagattgatctcgagctctcacataaagctagtgcaaacttgatcacatataccattttttcgtggtcaaatcgtttcccgccatacgcgtgtacatcggattaatcaatagctcgacgttgaccaagcttgttgacttgttcatcttcgttcctgtgcatcaaatcgttttattaattaattgagtcgatgtgacgccgcccatcgatcgaacactggtataatggaatgtatgggttgcccgccgtccccgtgcatatatgcatacgtgcaatgctttgctgccagatcttatctttcgaagaagaatcaacggaagaataatatcctcgctttattatattattgataacggtcaaccaaataaaaagccctgatgatgacttgatgagcaaactgcacaagtgtgttttgcattgcatgccaactgatgataccgtacgtggggtggtccatgatgcatgtgtgtgatccaaatccaacaatggcgcaggagctacttccggtgtcggcacaagctgtgcggcgccaagaagaaggtggagtggcacccgcgggaccccagcggcgacctccgcatcgtctacgagggcgcgcaccagcacggcgccccggcggcggcggctcctcccggtcccggcggccagcatcacggcggcggcgcctccgacttcaacagatacgagctgggcgcgcagtacttcggcggggccggccggtcgcattgacgcggggagccagggtcttgtttactttctaaaatattttataaaaattttcacattctttattacattaaattttgcggtacatacatgatgcactaaatatagataaaaaaaataactagttacatagtttatctgtcatttgtgagacgaatcttttgagcctaattagtttatgattgaacaatatttgtcaaatacaaacgaaagtattgacaaaccgacaagaaaggccggcggcgttgcgtcacgtacgcatgcatcagctcctgtgctggcctctgctggctgccgctgcatcgatcgatcgctttcgctgcgcaccggagggcagcggcaggtgctgccggtgccggttgacgccttgcgccggcgcaacgtgatgttgagtgcggattaattgttgctgctccggttaactctctggtctagtgctagtgtacggctactattaggacgatggtgcataattgtaattttgatattgtacatgcataaaaaacaatatttagctgaaagtgggaagtagcaccgtcgctattatgttttgttttctgcaaagtgtaaacttgtcgaaagtctccagagttgggttcgaggccctggtcacccagtttacattgcatcgcctctgaactgaatgcgacactcgagacctagctttatcagtgggatacacctaattcgtttagtgagcgtttaacattcaatcatttgcagataacctggcagctgacactgcaacggctgggtatccacaaccaacaagttggcaacactaataatgttttcgattgaggtaaacaccgaagagcggtaaacaaagttccatgcgatacgagacagctcgttcgcctagcaatctggaaagacacagtaataggcattcttatacagtacgtacaattcaaattattcatcctagcatacaacaacatcgaaaaagttaaaaaccacaagtgcaggaacatttggatacagaaacatgtctactgcgtggtcagtcgaccggttcctccatacggtgataataaccaacaagattattcccggtgtcctctacgatacagcatctcaaatacaacagataacttacaaccagtcacactcacacaatcccgtcagtagtcagtacattgccccagttacctacagtgccagtcttttcatcatcgcacagcactgaaagatactcagaaaagactttaatagactcgtgtctcaaagacaaagtagggcaaaatttatctactcttgttagcactcaagttaaccacatgggacacaaactactcaaactgaagtaatttgacaagtccaccagctaccacaacaaacccacccaaccatgacacaccgaggctcacagaaattacaggatgctataagttccgccagactttttatgtacagttagaatttatggtcacacaaaaaacctcaaggatgcttgtaattagaagaacgtgaccttcacttgggtcatctgcaaagagggcaccagaaggaaaagattagttttaaataattaattctagtactgcacacaccgacacgagttataaacaatataaacggtccatttggaatacagaaatttcacagaaatcatgtacaattccaagggaatcggtccattttcacaggaaaacacaggaaacagggggatcccacattccaaaaggggcttaaagagagaaggaattatcccacattacaggaattaacatgccatgacatctgatttgaatacctagaataccataataaaagtttgtttcgaaaacacagtagaaaacatgattccaacattttactatcaagtctaacaacaaataacatataggtgcccagtcccacacatgttccaaaaatgagtacaagacatagtgaacatagtcaacagaacaagagaatctcaattgcaggaagagtcatgcatgcgctatgattgaagcatgataaaaagaactacataccattgctgaacttttctaaactctgtcagtacaggatagatgttttcgaaggcagtgtaggtctcctctctcacctacaaacaatcgactatgaaattaaggagaaagataagctaatcgcagtataattaagcatgagcacgaaatgacaactaacctttgctccagtcaaaacaatcttgcctgaaacaaaaattaaaagaacaatctttggttgtttcatccgatagataaggccaggaaagagttctggttcgtactgtaaaacaaattaaaaatgtcattatccaaagaatgcagacaaaaaagggtaaaagaattactgtgatgttaaaataagccatcattggacatacacttgagaaggcaccatgagaatatgcaaggccctcaagcctaattggaaacttgacatcacaagagccaacaatattctgaatcttaaagtcctggcatagaacagtaacttagcaactgatgtacaaattgttcaaagtacaggtcaatgtacacaagtatgaaaatagttaccttaaatttagcaggaaaaccaagtttctgaataatacgagcatactggaaatacagacaggggttagaattccaaagctctcagtaaactagatccaacttaaataaaatggtagcaagccatatggcacctttcttgctgcaagcttagattgctgttcgctctttgctccagtacatacctggtgatagaaaattatcggttgcttgcttcagcactagaacacttatgatggattgatacaagattgtagttctatatgaaagaaatgcagttctagtaaactttcttcatttggaagaaaagtattgacacatcaatgcatttaattaatattcaatatgacaaccaagaaagtctacaatactgactattgatccaaataaatcccaagtaaaaacccaccgagatatatcatctggtaagggaaaatagatttgcctagggtaggctagagagggtaagaactttattctccaatatttgatgattgagagaggtagattaggacacagaaaaaacaaacagattagcctttctatcttttgacaggacagcaccaaggcaacaaaacatgtcaaaaaaaagatcaaatctgtttacatcaaaaacatgcaaaatccttgaaaattgacagtataagacaaaagatgttgatgacataccattttacccgatgcaaatatcagtgctgtggttttgggttcccttattctcatgatgactgcagcaaaacgctgtttacagataaaagagtcaaatacgaaatataatgacagaaaacttagcaaaattcaggttgctacattgtatcatcataactgagaaagattgcattcaatagaatgcctaaaagagcaaacaagtcatatataagctaaaaatttagaacttgattgtcaaagaatattgtggttattcacaggacaagcaggatatgagcatccatctggtttgaaactaaccgtgcacatctcatatcccaggccatccattagttattagcacaaagctatttgaactcatggacaagattgtacatcattacaaaggatcaacatactttatatgtccataaatcttccactagataaaaacaacaagtaaataccgtgcaaagccattgctttgaggtaatcactataccttggggttatactccgcatttcgtgcttgcaaagctattgctttgaggtcaagtttacaatccaaattaactgttgatacaatattcctgtcatgaaaaaatgacacacgtcaagcagaccatgatcaaagaactgcagtaaacatgtgaattttgttttgtaaaaccaacatagggttcttattgtaagtttttagcattgaagagacactacaagataattttcattgttctttttatatttgatagtgtgtgctattaatttcttcatgccaatttccaacatgtgcaaatcataataaatttaagactaacattcaagataacctacactataatggttggatcgtaaaatctttgtatcaatcaaagtcatttcaggactcaatatggcactaatatgcccatagcacttaataatgaaattgcctgcagaaaaatcttacacctaaatcataataaaaatcttccacaaaagctagttaggttacttctggtttggggacggagtgggatggaatggtcatgtccctattttttggacgggattgacccggatcttgtttggttggacagaaaggttcattccaatttttgtttggttcgaaggatatggtgggatggaacccgctggagttttaactccattagacacaataatccatggccgcaccatccattgtctctacacctgttcttgttgtcttcttcaggtgagcaaagcatgattcccaagattttgtaccacagtcgctcaacatctcacagctccggtgcccaacagctgggcactaccaccgcccaagagcttggccaacccattcgcccaagatctcatgcagagatcttggcattgccaccaccagagatgctcaacctgccccaccagagttctcatgtggccagaggaggtaattggacccactcctcttatcgtcggcgctagcccagtgggctgcatatgctccaaacatctcctctcctccgcttgccttgagcttggagcttccacgtgcctgcgcccctccttttgaccacgcttgcaccaggcaatgcaaagatggcgtgcaacgccgtccgcaaggaatggcttcatccacccgattcaaggggaccgagctgtccacatatttcaggaatatgccactgcaaaaaatgaccccatccctagctcctcccaaccaaacactgctgaaaaaggattgccccatcccgtctgggacgtccctcaatccaagccaatgcatttaaccctccccacgatataagatatggaaacctcagtgcgtgaggctgactgtttatcatattacacaatttatgcaccaacgagtcaaaacatagaatggaaatatggtaagaagagattatgcttgctgcaactattacgccaagatgacaaacttcaataaggaaatagatctcctctccagtttggccctctctcgttctcccaagtttcatacctgaaatcaaccctcggagagaggatgacaactaaataattcccaccaaagccccaactatttaagacaatattagctcgtttcgatgcacccagcaccgggaagctgaacaaaaacacggcataaaccaaccacaccaccacccacaagacagggaggcaccccgctggccagaaccaagccttggcagctccacagcacacccaagcacccatccgccgggcggcgggaccctagcacgtacggtacgggatctctccggaaccccgaatccccgacgacccagatccgggacttactggagcgtggggacgatgccggaggggtgcttggacagatccaccggctggctgccctcgagccccggctccgccatccgaaccacgcacgcgacctcggcggggctccgcgccgcgaatccggggccgaaatgggcgggaaaggagcgcgcgcgtcaccggttcgagggggaattcgaaatccgggtcttttatagagatcgggagaggagttggggaggagggaaagcaaggggaaggagagctagggttatctgtctcgcgagggggagtcggggacagcgcaggcggcgtgagaatgcggggggaagagggggaggtcgtctggtggtgggaggtagatgcgtgcgggagttggggttgtatcggtggacggggagcaggcggtggatggcgactgcttggcttttgtaggggaacagggtgcaccggctgtggccggttaccccagggcgcggtttgcccacgcgctggttcgagttatgcaaactgacctgtgggtcatagcatgcggtgggacccggtgtcggtgtgtgtgggtatgatgcgcgttcgacggccattaatcaagaatttctcctactcgcagatcgcactagcaggtttacgaacgcgccgagaagatcgcactattatgaattattttctttgaaagaaaattgttatgaattatgaaaatcatgaactatactaatcggactatttgaattattgtgatggatcattttccgttcgagtgggaaatcatggtcaccaaaaagctggtaagagagagattataagatgattattatagtcgagtgttttagttatgtttagtttataattaaattattttagctaattattataatcacagtggatccaaacaggcctgactagtgactacttgagcattcgcgttacgtcgctgttgcagtgcacattcattaatgttaaggccttgtttagttcccagaatattttgtaaaaattttcagattcttccatcacatcgaatcttgcggcatatgtatggagcactaaatatagatgaaagaaataactaattacataatttatctgtaatttgtgagatgaatcttttgagtctaattagtctatgattagataatatttgttaaatacaaacgaaagtgctattgttcctattttgcaaaaaaatttgaaactaaacaaggcctaactaaaacatcttgcgttagagcttccttgatgcaccacggtggcgtgctgtcgtagtgaccacctcagctctagacttccatgtcataggctcttgcagaggagatcatggcctcatctaaaaaaaatcaaaggcaacagctaggcagcgtgctatggtggaagtagtggctctaagctattgggaccacgtctggttcgtgcatttggctccaaattgtctttagcagcgactgacggtggaacgcctatagagacaagccacatgcagcttgcattgagtacaatggtggttttaacttttaacccatcgaacgtacgtggatggtcacctttttttcctggggctaacgctactaggtgcccgtgttgcgactacccttaggctgtctccaaaggcatgtgaaatttttttggatttcgctactgtagcactttcgtttgtttgtgataaatattgttcaataatagactaactagggttaaaaaatttgtctcacgatttacagtcaaactgtgtgattagtttttgttttcgtctatatgcttcatgcatttgccgcaaaattcgatgtgacagggaatcttgaaatttttttggatttcagaattaactaaacaaggcccaagacccatttgggaacccaaatccaaaataggttttcaacacaatacctatagcctccaacagagtactcatacagaagatccattttgagtatcaggagaggcataacccaaatttgggtatcctctctcttcgagacccatttgtagagagtgttgtcttttaggtcttgttgttggaaaagactaaaaataggtatggatccttttagctgtagcgctaaccaaatgacaaatgagttttgtattttgggtgacgattgttgaagacagtcttgtactagccacaacggcgagcatcgatgtgtcagtaagcatgtcagtaagcatcggtttataagagagctgtaatgtctaaacatcatgtgggaccaaccaaatgaataaacaaacaaggagacattgcaatgcctgaacatatcagtgagcatcggttgaaactcgccctctctcagtatgtgcaactatagtttttttatgttgcactgtggaaagtagaagcctcgatgtcgcacaaaaaaaaatcagcatcgcaccccgcgatgtgatgcctcaaggctagaagccaaaatatgcgcaatggtaaaactatacgttatgtgtagtcttatatataaaatgttagaaaaaaatatttcattttagaatggagagagtaggcaataagaccagtacaaaacggacataaatctaaaacaaatattgtttgagagaaaatatctaaaatcaatccaagtataagcaagcatcatatgtgacataataagagattaataatcctaaaatgagtgtacatgtcttgcatcaatttatgaaactcgaattatctgtctcccagagcacgagccaatgccactcataacctattacatataggtcaatcttttacagagcttgtgatcatctttatatctgatcatcatttaacgatctgcgggactagtaggctatcagaagcaataaccttcggttgtttcagatggacacgaatgtgcatcaccagtttacagctctgtatacttcacctaataactgaacattctgagagaatgaactatttgtggctccttgatgaggcccagcatgtttaccttttaggttcccttaggttaaacactaaatcttcatgatggaaggtgtttgcctgaactccaagacagcaaggttttctctatacttctttacttcggccaccattctgtcgtacgattcagggtatttgcaaaaaatcacgattttgattcagctccctggctcgtgcctgcaatgtcaacatgatcctttacaaatgttcgaaggcatccattaattacccgaggggcaccaccatcacaaaatcgctttgccagatctactgcctgaaagacaagggtcgagagacatttatattctactagtactcaaaagtggaaagagtaatagctataagaaaacatgcaggtgcttgatgcataaagtcaaaatatgaagaaaaacaagtaatagggagaaataagcacctcattgatgacaactttgtgaggtgttccttttgatgtcatctctgccatagcaatatgtagaatgcagagctcaagtatccttgccacaggctcatcctgccatgaaattttccatgtatcaacagcaggttatgccataaaacaagacagcaaaataataaatactaaaatataacaccaagttaaagatcaggaagattataaactgatgaaaggaaagtaatatattgtgtttgaaccaaacacaatataaacagcttgatgcatatcgaagggatttgatgaatcaacatagaatagtaggaaaaggtatctaaccttccaagcctggggaattattttgtcaatgatatctacatgcttatcccatccactagcaacagccactaaaagttccctggacaacctgtactggaaaaatatctaattaggaatgtaagagcagcaggactaaatattaaacaggaaattaaattttatcatatatcagaacagtgtatcgatacctaatgcctttagtggaattgggcaagaaggaaagtataccgtaagacaaagttgttgtacaccagttttggaggagctgaaagtacatcttcttctgaatatgaaagaaaaacatgtcaaattctttgcagaagaataaccaaacattaatggaacatatttacacaaaaacaaatctatagttactcagctgatttcacaacagactaaggaagaaaatgtatacggttaatatgactatatgagccgtttagcacgcatcgtaaggatatgtttattgtgctgaacgagatagatgccactgggctgctacaaaagatgcatgctaacgaacgtgaacagttttcagcatgtcgattaaaagtgtaatcaacacatagcttgataaaatatatcaaaatttactggcgcttagagtgatggattatggtatagctctcttaaaactcagtctgcaaaaccaccaaaagaaaaaaaaaacagatacacaacccctgtagatcttaatgacctagcctgactaggtagcacctaggcattagccactataccgaatcaagagttaggtgccacacagctgcttacctagcacattgggttttttaagccaaagcactgcattaactgttgtagtttaacggtctgaaattcacagcaccaactgtgaattgctctagcatgccctccagtttttatatacatgaaaataggcacacgcccacaataaaaaaaaaaagaaacttggcctaagttcaataacgtatttatggaacaaccaatgatccattgctctctttactttaggaaaccagaatcatagatatatgacgaaagtttcaaaacttagactgaaacccaccataaaatttgtttaaacaggaaccaactagattttctggtggttgtatgtttcagattgaccgaaggataaccattaaaagactgctataatggaattggtacctaactgaacttgtgctctttggaatcttctggatatagaaatattgaatctcaaaattgtgaaaaaaaaagatgggcatatgtccaaatttaccaacaacaatctacgactccaactgtaacagcgttaacatataggaagtagctatgttaccccgatacttctctgaatcgccgtaccgatatcgcgatacgtatccgatacggcgccgatacggtatcggagaagtatcgaggaaatagagaaataaaaataaataaaataaatccgatactagaccgataccttcccgatacttcccagcccataacctctcaaattgaagtccatcaagttagcagctcatttttgtggcccatttacacaacactaaaaccctactagccaccacacgtacacaatagatgtagtagcggacttagcctaaaacttatagtatcctaatatttatttttctgctgtaaggatattaaaaacaatatttagttttctgctggtgtgaaaccaaata

SEQ ID NO: 12 - protein, 144 aa, Sorghum propinquum
MDSSSQPGAIDTCRGSGGGGDRNQREEDAAAAAAAEAGYGRQLVIPEDGYEWKKYGQKFIKNIQKIRSYFRCRHKLCGAKKKVEWHPRDPSGDLRIVYEGAHQHGAPAAAAPPGPGGQHQGGGASDFNRYELGAQYFGGAGRSH

SEQ ID NO: 13 - protein, 200 aa, Sorghum propinquum
MAEPGLEGSQPVDLSKHPSGIVPTLQNIVSTVNLDCKLDLKAIALQARNAEYNPKRFAAVIMRIREPKTTALIFASGKMVCTGAKSEQQSKLAARKYARIIQKLGFPAKFKDFKIQNIVGSCDVKFPIRLEGLAYSHGAFSSYEPELFPGLIYRMKQPKIVLLIFVSGKIVLTGAKVREETYTAFENIYPVLTEFRKVQQ

SEQ ID NO: 14 - protein, 44 aa, Sorghum propinquum
MDSSSQPGAIDTCRGSGGGGDRNQREEDAAAAAAAEAGYGRQLV

SEQ ID NO: 15 - protein, 100 aa, Sorghum propinquum
IPEDGYEWKKYGQKFIKNIQKIRSYFRCRHKLCGAKKKVEWHPRDPSGDLRIVYEGAHQHGAPAAAAPPGPGGQHQGGGASDFNRYELGAQYFGGAGRSH

SEQ ID NO: 16 - protein, 100 aa, Sorghum bicolor
MPEDGYEWKKYGQKFIKNIQKIRSYFRCRHKLCGAKKKVEWHPRDPSGDLRIVYEGAHQHGAPAAAAPPGPGGQHHGGGASDFNRYELGAQYFGGAGRSH

SEQ ID NO: 17 - protein, 201 aa, Sorghum bicolor
MAEPGLEGSQPVDLSKHPSGIVPTLQNIVSTVNLDCKLDLKAIALQARNAEYNPKRFAAVIMRIREPKTTALIFASGKMVCTGAKSEQQSKLAARKYARIIQKLGFPAKFKDFKIQNIVGSCDVKFPIRLEGLAYSHGAFSSYEPELFPGLIYRMKQPKIVLLIFVSGKIVLTGAKVREETYTAFENIYPVLTEFRKVQQC

SEQ ID NO: 18 - protein, 142 aa, Oryza sativa
MNNRGAGGGRGQGDDEEPTPTPPPAPPPETAPSTVGGGGDGVQLVMPEDGYEWKKYGQKFIKNIQKNRSYFRCRDQRCGAKKKVEWHPHDPGLNLRVVYDGAHHHGSPSSAAGEGGTSAAAAANQYDLSTQYFGGAGGPRSQ

SEQ ID NO: 19 - protein, 99 aa, Zea mays
MPEDGYEWKKYGQKFIKNIQKMRSYFRCRHRLCGAKKKVEWHPRDPGGDLRIVYDGAHQHGPPPLAAPPGGHGQMQGGGASDSNRYELCTQYFGGARSR

SEQ ID NO: 20 - protein, 140 aa, Zea mays
MDDSSSQPATIVSGGEERNQRREEDDDEETAAAEAEAGNGSQLMVVPEDGYEWKKYGQKFIKNIQKIRSYFRCRHRLCGAKKKVEWHPRDPSAALRVVYDGAHQHGAAPPAAPEQGGGGGASNRYELGAQYFGAGPAGSG

SEQ ID NO: 21 - protein, 132 aa, Setaria italica
MASSSQHPAAIGSGGGDKNQAEDAAAVPAAAASEAGGQLVMPEDGHEWKKYGQKFIKNIQKIRSYFRCRHRLCGAKKKVEWHPSDPSGALRVVYEGAHQHGSPSPPSSAAGGASNRYELGAQIFGGARSQSQ

SEQ ID NO: 22 - protein, 93 aa, Setaria italica
MPQDGYEWKKYGQKFIKNIQKNRSYFRCRHKLCGVKKKVEWHPSDPAGDLRIVYEGAHQHGSGAAPSSAKGAAAGASNQYELGAQYFGGARSH

SEQ ID NO: 23 - protein, 152 aa, Brachypodium distachyon
MGLLEHHLRVYDADETINLSVLNHWFAPLISEGVGEGAGHGDEEDQHAAEAAASSGQQLVMPEDGYEWKKYGQKFIKNIQRIRSYFRCRDKRCGAKKKVEWQPGDPSLRVVYEGAHQHGSPSSSSSCTGAGHDGAANRYELSAQYFGGPRTQ